(19) United States
(12) Patent Application Publication
(10) Pub. No.: US 2024/0041757 A1
(43) Pub. Date: Feb. 8, 2024

(54) LIPID NANOPARTICLES (LNPs)-BASED OCULAR DELIVERY

(71) Applicant: CRISPR Therapeutics AG, Zug (CH)

(72) Inventors: Seshidhar Reddy Police, South Boston, MA (US); Dominique Ouellet, South Boston, MA (US); Michael Lukason, South Boston, MA (US); Mary-Lee Dequeant, South Boston, MA (US)

(21) Appl. No.: 18/336,961

(22) Filed: Jun. 17, 2023

Related U.S. Application Data
(60) Provisional application No. 63/353,374, filed on Jun. 17, 2022.
(60) Provisional application No. 63/417,233, filed on Oct. 18, 2022.

(51) Int. Cl. A61K 9/00; C12N 9/22; C12N 15/11; A61P 27/06; A61K 47/69

(52) U.S. Cl. CPC A61K 9/0048; C12N 9/22; C12N 15/11; A61P 27/06; A61K 47/6909; C12N 2310/20; C12N 2800/80; C12N 2320/35

(57) ABSTRACT

Provided herein include compositions, methods and systems for delivery of CRISPR/Cas-mediated gene editing systems using lipid nanoparticles (LNP) to trabecular meshwork cells. Methods, compositions and systems for treating glaucoma are also provided herein, which involve reducing the expression of myocilin (MYOC) gene in the trabecular meshwork cells of patients' eyes.

### CROSS-REFERENCE TO RELATED APPLICATIONS

This application claims the benefit under 35 U.S.C. § 119(e) of U.S. Provisional Patent Application No. 63/353,374, filed Jun. 17, 2022, and U.S. Provisional Patent Application No. 63/417,233, filed Oct. 18, 2022, the contents of which are incorporated by reference herein in their entireties.

### REFERENCE TO SEQUENCE LISTING

The present application is being filed along with a Sequence Listing in electronic format. The Sequence Listing is provided as a file entitled 80EM-341773-US_SeqList, created Jun. 16, 2023, which is 479 kilobytes in size. The information in the electronic format of the Sequence Listing is incorporated herein by reference in its entirety.

### BACKGROUND

### Field

The present disclosure generally relates to the field of molecular biology and biotechnology, including gene editing.

### Description of the Related Art

Treatment of eye diseases (e.g., glaucoma) requires accurate and efficient ocular delivery of therapeutics, for example delivery to the trabecular meshwork cells of the patients. Mutations in the myocilin (AYOC) gene that encodes myocilin are causative for some forms of juvenile and adult-onset primary open-angle glaucoma (POAG). Myocilin is a secreted 55-57 kDa glycoprotein that forms dimers and multimers. It has a myosin-like domain, a leucine zipper region and an olfactomedin domain. Most of the mutations that have been identified in patients with POAG are localized in the olfactomedin domain, which is highly conserved among species. In the eye, myocilin is expressed in high amounts in the trabecular meshwork (TM), sclera, ciliary body and iris, and at considerably lower amounts in the retina and the optic nerve head (Tamm, Prog Retin Eye Res. 2002 July; 21(4): 395-428).

Glaucoma is a group of progressive optic neuropathies characterized by degeneration of retinal ganglion cells and resulting changes in the optic nerve head. There are several types of glaucoma, including POAG, angle-closure glaucoma, congenital glaucoma, and normal-tension glaucoma. Loss of ganglion cells is related to the level of intraocular pressure (IOP), but other factors may also play a role. Reduction of IOP is the only proven method to treat the disease. Although treatment is usually initiated with ocular hypotensive drops, laser trabeculoplasty and surgery may also be used to slow disease progression (Weinreb, et al., JAMA. 2014 May 14; 311(18): 1901-1911).

### SUMMARY

Disclosed herein includes a method for delivering a CRISPR/Cas-mediated gene editing system to cells of the eye of a subject, the method comprising administering to the subject a plurality of lipid nanoparticles (LNPs) complexed with (a) a guide RNA for a target gene or a nucleic acid encoding the guide RNA; and/or (b) a RNA-guided endonuclease or a nucleic acid encoding the RNA-guided endonuclease, thereby reducing the expression of the target gene in cells of the eye of the subject. For example, the CRISPR/Cas-mediated gene editing system can be delivered to the trabecular meshwork cells of the subject. The target gene can be, for example myocilin (MYOC) gene (e.g., a wildtype MYOCor a mutant MYOC gene). In some embodiments, the expression of the target gene, the expression of the protein encoded by the target gene, or both, in the subject's eye is reduced by at least 20%, by at least 40%, by at least 70%, or by at least 90% after the administration. In some embodiments, the expression of the target gene is reduced in the trabecular meshwork cells of the subject's eye.

Also disclosed herein includes a method for treating a subject with glaucoma, the method comprising administering to the subject a plurality of lipid nanoparticles (LNPs) complexed with (a) a guide RNA targeting myocilin (MYOC) gene or a nucleic acid encoding the guide RNA; and (b) a RNA-guided endonuclease or a nucleic acid encoding the RNA-guided endonuclease, thereby reducing expression of MYOC gene in the subject's eye. In some embodiments, the glaucoma is myocilin-associated glaucoma. The glaucoma can be, for example, primary open-angle glaucoma (POAG). In some embodiments, the expression of the MYOC gene is reduced in the trabecular meshwork cells of the subject's eye.

In the method described herein, the RNA-guided nuclease can be a Cas9 nuclease, for example a Slaphy/ococcus aureus Cas9 (SaCas9) nuclease or a Streplococcus pyogenes Cas9 (SpCas9) nuclease. In some embodiments, the site targeted by the guide RNA is within exon 1, exon 2 or exon 3 of the MYOC gene. In some embodiments, the site targeted by the guide RNA comprises a nucleotide sequence selected from the group consisting of SEQ ID NOs: 1-27 and 55-115. In some embodiments, the site targeted by the guide RNA comprises a nucleotide sequence selected from the group consisting of SEQ ID Nos: 6, 10, 15, 18, 26, 59, 61, 63, 64, 66, 69, 72-77, 79, 81, 82, 90, 95, 98-101, 104, 106, 107, 109, and 113-115. In some embodiments, the site targeted by the guide RNA comprises a nucleotide sequence selected from the group consisting of SEQ ID Nos: 10, 64, 73, 74, 75, 76, and 115. In some embodiments, the guide RNA comprises a spacer sequence having a RNA sequence corresponding to any one of the nucleotide sequence selected from the group consisting of SEQ ID NOs: 1-27 and 55-115. In some embodiments, the guide RNA comprises a spacer sequence having a RNA sequence corresponding to any one of the nucleotide sequence selected from the group consisting of SEQ ID NOs: 6, 10, 15, 18, 26, 59, 61, 63, 64, 66, 69, 72-77, 79, 81, 82, 90, 95, 98-101, 104, 106, 107, 109, and 113-115. In some embodiments, the guide RNA comprises a spacer sequence having a RNA sequence corresponding to any one of the nucleotide sequence selected from the group consisting of SEQ ID NOs: 10, 64, 73, 74, 75, 76, and 115. The guide RNA can be, for example, a SaCas9 sgRNA or SpCas9 sgRNA.

In some embodiments, the guide RNA comprises a nucleotide sequence selected from SEQ ID NOs: 195-371. In some embodiments, the guide RNA comprises a nucleotide sequence selected from SEQ ID NOs: 258, 267-270, 309, 319, 328-331, 370 and 371.

In some embodiments, a LNP of the plurality of LNPs comprises an ionizable cationic lipid, a helper lipid, a sterol, and a poly(ethylene glycol)-lipid (PEG-lipid). The LNP can comprise about 20-60% the ionizable lipids, about 18.5% to 60% the sterol, about 0.01 to 30/a the helper lipid, and/or about 0%-10% PEG-lipid. In some embodiments, the ionizable cationic lipid is selected from C12-200, cKK-E12, DLIN-MC3, DLIN-MC4, DLIN-MC5, DODMA, DOTAP, DODAP, DC Cholesterol, DLin-DMA, DLin-K-DMA, and DLin-KC2_DMA. In some embodiments, the helper lipid is selected from 1,2-distearoyl-sn-glycero-3-phosphocholine (DSPC), 1,2-dioleoyl-sn-glycero-3-phosphocholine (DOPC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-phosphocholine (DOPC), diundecanoylphosphatidylcholine (DUPC), phosphatidylcholine (POPC), 1-stearoyl-2-oleoyl-sn-glycero-3-phosphocholine (SOPC), 1,2-dioleoyl-Sn-glycero-3-phosphoethanolamine (DOPE), 1,2-distearoyl-sn-glycero-3-phosphoethanolamine (DSPE), 1,2-dihexadecanoyl-sn-glycero-3-phosphoethanolamine, (DPPE), 1,2-dimyristoyl-sn-glycero-3-phosphoethanolamine (DMPE), 1-stearoyl-2-oleoyl-sn-glycero-3-phosphoethanolamine (SOPE, 18:0-18:1 PE), 1-palmitoyl-2-oleoyl-sn-glycero-3-phosphoethanolamine (POPE), dioleoyl phosphatidylglycerol (DOPG), and dipalmitoyl-sn-glycero-3-PG (DPPG). In some embodiments, the sterol is selected from cholesterol, sitosterol, 8-sitosterol, phytosterols, fucosterol, zoosterol, and ergosterol. In some embodiments, the sterol is selected from cholesterol, sitosterol, campesterol, stigmasterol, fucosterol, and ergosterol. In some embodiments, the PEG-lipid is DMG-PEG, DSG-PEG, a PEG-ceramide, or a PEG-phospholipid. In some embodiments, a LNP of the plurality of LNPs comprises about 50 mol % of C12-200, DLIN-MC3, DODMA or DOTAP, about 10 mol % of DSPC, about 37.0-39.5 mol % of cholesterol or sitosterol, and about 0.5-3.0% of DMG-PEG. In some embodiments, the LNP comprises about 50 mol % of C12-200, about 10 mol % of DSPC, about 37.0-39.5 mol % of sitosterol, and about 0.5-1.5% of DMG-PEG. In some embodiments, the average particle size of the plurality of LNP is about 80-100 nm, and optionally 85-95 nm.

The plurality of LNP can be administered to the subject, for example, by intravitreal injection or intracameral injection. In some embodiments, the method comprises a single administration of the plurality of LNPs to the subject. In some embodiments, the MYOC expression in the subject's eye is reduced by at least 20%, by at least 40%, by at least 70%, or by at least 90% after the administration. In some embodiments, the myocilin protein in the trabecular meshwork cells of the subject's eye is reduced by at least 20%, by at least 40%, by at least 70/0, or by at least 90% after the administration.

As described herein, the subject can be a human. In some embodiments, the LNPs are complexed with (a) the guide RNA or a nucleic acid encoding the guide RNA and (b) the RNA-guided endonuclease or the nucleic acid encoding the RNA-guided endonuclease separately. In some embodiments, the LNPs complexed with (a) the guide RNA or a nucleic acid encoding the guide RNA and the LNPs complexed with (b) the RNA-guided endonuclease or the nucleic acid encoding the RNA-guided endonuclease are different LNPs.

Disclosed herein includes a guide RNA targeting a MYOC gene, comprising a nucleotide sequence specific to a fragment in exon 1, exon 2 or exon 3 of the MYOC gene, wherein the guide RNA comprises a spacer sequence having a RNA sequence corresponding to any one of the nucleotide sequence selected from SEQ ID NOs: 1-27 and 55-115 or a spacer sequence having one, two, or three mismatches relative to a RNA sequence corresponding to any one of the nucleotide sequence selected from SEQ ID NOs: 1-27 and 55-115. In some embodiments, the guide RNA comprises a spacer sequence having a RNA sequence corresponding to any one of the nucleotide sequence selected from SEQ ID NOs: 6, 10, 15, 18, 26, 59, 61, 63, 64, 66, 69, 72-77, 79, 81, 82, 90, 95, 98-101, 104, 106, 107, 10 In some embodiments, the guide RNA comprises a spacer sequence having a RNA sequence corresponding to any one of the nucleotide sequence selected from SEQ ID NOs: 10, 64, 73, 74, 75, 76, and 115. In some embodiments, the guide RNA comprises a nucleotide sequence selected from SEQ ID NOs: 195-371. In some embodiments, the guide RNA comprises a nucleotide sequence selected from SEQ ID NOs: 258, 267-270, 309, 319, 328-331, 370 and 371.

Also disclosed herein includes a system for treating a subject with a glaucoma, the system comprising: (i) a gene editing means that targets reducing expression of myocilin (MYOC) gene in the subject's eye; and (ii) a lipid nanoparticle (LNP), wherein the LNP delivers the gene editing means to the subject's eye. In some embodiments, the glaucoma is myocilin-associated glaucoma.

The glaucoma can be primary open-angle glaucoma (POAG). In some embodiments, the gene editing means is CRISPR/Cas-mediated gene editing. In some embodiments, the CRISPR/Cas-mediated gene editing comprises an RNA-guided nuclease and a guide RNA targeting a site in the MYOC gene. In some embodiments, the RNA-guided nuclease is a Cas9 nuclease, e.g., a Slaphylococcus aureus Cas9 (SaCas9) nuclease or a Slreplococcus pyogenes Cas9 (SpCas9) nuclease.

In some embodiments, the site targeted by the guide RNA is within exon 1, exon 2, and exon 3 of the MYOC gene. In some embodiments, the site targeted by the guide RNA comprises a nucleotide sequence selected from the group consisting of SEQ ID NOs: 1-27 and 55-115. In some embodiments, the site targeted by the guide RNA comprises a nucleotide sequence selected from the group consisting of SEQ ID NOs: 64, 73, 74, 75, 76, and 115.

The guide RNA can be a SaCas9 sgRNA or SpCas9 sgRNA. In some embodiments, the guide RNA comprises a nucleotide sequence selected from the group consisting of SEQ ID NOs: 195-371. In some embodiments, the guide RNA comprises a nucleotide sequence selected from the group consisting of SEQ ID NOs: 258, 267-270, 309, 319, 328-331, 370 and 371. In some embodiments, the LNP comprises an ionizable cationic lipid, a helper lipid, a sterol, and a poly(ethylene glycol)-lipid (PEG-lipid). In some embodiments, the LNP comprises about 20-60% the ionizable cationic lipids, about 18.5% to 60% the sterol, about 0.01 to 30% the helper lipid, and/or about 0/6-10% PEG-lipid. In some embodiments, the ionizable cationic lipid is selected from C12-200, cKK-E12, DLIN-MC3, DLIN-MC4, DLIN-MC5, DODMA, DOTAP, DODAP, DC Cholesterol, DLin-DMA, DLin-K-DMA, and DLin-KC2_DMA. In some embodiments, the helper lipid is selected from 1,2-distearoyl-sn-glycero-3-phosphocholine (DSPC), 1,2-dioleoyl-sn-glycero-3-phosphocholine (DOPC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-phosphocholine (DOPC), diundecanoylphosphatidylcholine (DUPC), phosphatidylcholine (POPC), 1-stearoyl-2-oleoyl-sn-glycero-3-phosphocholine (SOPC), 1,2-dioleoyl-Sn-glycero-3-phosphoethanolamine (DOPE), 1,2-distearoyl-sn-glycero-3-phosphoethanolamine (DSPE), 1,2-dihexadecanoyl-sn-glycero-3-phosphoethanolamine, (DPPE), 1,2-dimyristoyl-sn-glycero-3-phosphoethanolamine (DMPE), 1-stearoyl-2-oleoyl-sn-glycero-3-phosphoethanolamine (SOPE, 18:0-18:1 PE), 1-palmitoyl-2-oleoyl-sn-glycero-3-phosphoethanolamine (POPE), dioleoyl phosphatidylglycerol (DOPG), and dipalmitoyl-sn-glycero-3-PG (DPPG).

In some embodiments, the sterol is selected from cholesterol, sitosterol, phytosterols, fucosterol, zoosterol, and ergosterol. In some embodiments, the sterol is selected from cholesterol, sitosterol, β-sitosterol, campesterol, stigmasterol, fucosterol, and ergosterol. In some embodiments, the PEG-lipid is DMG-PEG, DSG-PEG, a PEG-ceramide, or a PEG-phospholipid.

In some embodiments, the LNP comprises about 50 mol % of C12-200, DLIN-MC3, DODMA or DOTAP, about 10 mol % of DSPC, about 37.0-39.5 mol % of cholesterol or sitosterol, and about 0.5-3.0% of DMG-PEG. In some embodiments, the LNP comprises about 50 mol % of C12-200, about 10 mol % of DSPC, about 37.0-39.5 mol % of sitosterol, and about 0.5-1.5% of DMG-PEG. In some embodiments, the system is administered to the subject by intravitreal injection or intracameral injection.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIGS. 1A-1B illustrate quantifications of editing efficiency of SaCas9 sgRNAs targeting MYOC coding sequence as ranked by guide numbering (FIG. 1A) or by total indel percentages (FIG. 1B).

FIGS. 2A-2B illustrate quantifications of editing efficiency of SpCas9 sgRNAs targeting MYOC coding sequence as ranked by guide numbering (FIG. 2A) or by total indel percentages (FIG. 2B).

FIGS. 3A-3B illustrate Source 1 LNP screen in primary trabecular meshwork (TM) cells in culture (source 1). FIG. 3A includes LNP screen pictures. FIG. 3B is a graph showing LNP screen GFP scores.

FIGS. 4A-4B illustrate Source 2 LNP screen in the immortalized GTM3 cell line. FIG. 4A includes LNP screen pictures. FIG. 4B is a graph showing LNP screen GFP score.

FIGS. 5A-5B illustrate Source 2 LNP screen in primary TM cells (source 1). FIG. 5A includes LNP screen pictures. FIG. 5B is a graph showing LNP screen GFP score.

FIGS. 6A-6B illustrate Source 2 LNP screen in primary TM cells (source 2). FIG. 6A includes LNP screen pictures. FIG. 6B is a graph showing LNP screen GFP score.

FIG. 7 illustrates quantification of editing efficiency of selected SpCas9 sgRNAs targeting MYOC coding sequence using selected lipid nanoparticles for delivery in the immortalized GTM3 cells.

FIG. 8 illustrates Source 1 LNP screen GFP score in mouse trabecular meshwork.

FIG. 9 illustrates Source 2 LNP screen GFP score in mouse trabecular meshwork.

FIGS. 10A-10C illustrate exemplary embodiments of GFP protein expression in the trabecular meshwork tissue after delivery of different LNPs/eGFP mRNA in vivo in mouse eye.

FIG. 10A: IHC showing GFP protein expression in mouse trabecular meshwork after delivery of Source 1 CTX-C12-CT. FIG. 10B: IHC showing GFP protein expression in the mouse trabecular tissue after intravitreal injection of LNP CTX-C12-200-CT/eGFP mRNA in a mouse eye. FIG. 10C: IHC showing GFP protein expression in the mouse trabecular tissue after intravitreal injection of LNP A14/eGFP mRNA in a mouse eye.

FIG. 11 illustrates an exemplary embodiment of MYOC gene editing and myocilin protein knockdown after delivery of LNP CTX-C12-200-CT/Cas9 mRNA/SpMCh10 sgRNA to the human glaucomatous trabecular meshwork cell line GTM3 MYOC^{Y437H}-dsRED. Panel A: Epifluorescence microscopy showing a dose-response decrease of MYOC^{Y437H}-dsRED protein expression from LNP-transfected GTM3 MYOC^{Y437H}-dsRED after 7 days. Panel B: Immunoblot showing a decrease of MYOC^{Y437H}-dsRED protein expression from lysates of LNP-transfected GTM3 MYOC^{Y437H}-dsRED cells after 5 days. Panel C: Normalized expression of the Myocilin^{Y437H}-dsRED protein from analyzed immunoblot data shown in Panel B. Panel D: Editing percentages at both genomic and transgenic MYOC locus in LNP-transfected GTM3 MYOC^{Y437H}-dsRED after 5 days.

FIG. 12 illustrates an exemplary embodiment of MYOC gene editing and myocilin protein knockdown after delivery of LNP CTX-C12-200-CT/Cas9 mRNA/SpMCh10 sgRNA to human primary trabecular meshwork cells. Panel A: Editing percentages at genomic MYOC locus in LNP transfection in human primary trabecular meshwork cells. Panel B: Immunoblot showing reduced expressing of myocilin in high edited samples treated with dexamethasone. Panel C: Normalized expression of the myocilin protein from analyzed immunoblot data shown in Panel B.

FIG. 13 illustrates an exemplary embodiment of an IHC showing GFP protein expression in the trabecular meshwork tissue after delivery of LNP CTX-C12-200-CT/eGFP mRNA in an ex vivo anterior segment organ culture (ASOC).

FIG. 14 illustrates an exemplary embodiment of MYOC gene editing and myocilin protein knockdown in the trabecular meshwork tissue after delivery of LNP CTX-C12-200-CT/Cas9 mRNA/MYOC sgRNAs in an ex vivo anterior segment organ culture (ASOC). Panel A: Editing percentage at genomic MYOC locus in LNP-perfused ASOC after 4 days from DNA isolated from cornea, sclera and trabecular meshwork tissues. Panel B: Immunoblot showing a decrease of myocilin protein expression from donor 1656 trabecular meshwork lysate of LNP-perfused ASOC after 4 days. Panel C: Normalized expression of the myocilin protein from analyzed immunoblot data shown in Panel B.

FIG. 15 illustrates α-SMA protein expression knockdown in the mouse trabecular meshwork after delivery of LNP CTX-C12-200-CT/Cas9 mRNA/ACTA2 gRN A in vivo in mouse eye. Panel A. α-SMA protein expression IHC score from naïve (untreated) and treated animals 1-2- and 4-weeks post LNP injection. Panel B. Example of α-SMA protein expression IHC score of 3 in the trabecular meshwork from naïve (untreated) animals. Panel C. Example of α-SMA protein expression IHC score of 0 in the trabecular meshwork from treated animals 4 weeks post LNP injection.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods, systems, compositions and kits for treating a subject with glaucoma. The method involves reducing the expression of myocilin gene in the trabecular meshwork cells of the subject's eye.

### Definitions

As used herein, the term “about” means plus or minus 5% of the provided value.

As used herein, the term “RNA-guided endonuclease” refers to a polypeptide capable of binding a RNA (e.g., a gRNA) to form a complex targeted to a specific DNA sequence (e.g., in a target DNA). A non-limiting example of RNA-guided endonuclease is a Cas polypeptide (e.g., a Cas endonuclease, such as a Cas9 endonuclease). In some embodiments, the RNA-guided endonuclease as described herein is targeted to a specific DNA sequence in a target DNA by an RNA molecule to which it is bound. The RNA molecule can include a sequence that is complementary to and capable of hybridizing with a target sequence within the target DNA, thus allowing for targeting of the bound polypeptide to a specific location within the target DNA.

As used herein, the term “guide RNA” or “gRNA” refers to a site-specific targeting RNA that can bind an RNA-guided endonuclease to form a complex, and direct the activities of the bound RNA-guided endonuclease (such as a Cas endonuclease) to a specific target sequence within a target nucleic acid. The guide RNA can include one or more RNA molecules.

As used herein, a “secondary structure” of a nucleic acid molecule (e.g., an RNA fragment, or a gRNA) refers to the base pairing interactions within the nucleic acid molecule.

As used herein, the term “Cas endonuclease” or “Cas nuclease” refers to an RNA-guided DNA endonuclease associated with the CRISPR adaptive immunity system.

Unless otherwise indicated “nuclease” and “endonuclease” are used interchangeably herein to refer to an enzyme which possesses endonucleolytic catalytic activity for polynucleotide cleavage.

As used herein, the term “invariable region” of a gRNA refers to the nucleotide sequence of the gRNA that associates with the RNA-guided endonuclease. In some embodiments, the gRNA comprises a crRNA and a transactivating crRNA (tracrRNA), wherein the crRNA and tracrRNA hybridize to each other to form a duplex. In some embodiments, the crRNA comprises 5′ to 3′: a spacer sequence and minimum CRISPR repeat sequence (also referred to as a “crRNA repeat sequence” herein); and the tracrRNA comprises a minimum tracrRNA sequence complementary to the minimum CRISPR repeat sequence (also referred to as a “tracrRNA anti-repeat sequence” herein) and a 3′ tracrRNA sequence. In some embodiments, the invariable region of the gRNA refers to the portion of the crRNA that is the minimum CRISPR repeat sequence and the tracrRNA.

As used herein, the term “donor template” refers to a nucleic acid strand containing exogenous genetic material which can be introduced into a genome (e.g., by a homology directed repair) to result in targeted integration of the exogenous genetic material. In some embodiments, a donor template can have no regions of homology to the targeted location in the DNA and can be integrated by NHEJ-dependent end joining following cleavage at the target site. A donor template can be DNA or RNA, single-stranded or double-stranded, and can be introduced into a cell in linear or circular form.

As used herein, the terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases (e.g., the five biologically occurring bases adenine, guanine, thymine, cytosine and uracil) or other natural, chemically or biochemically modified, non-natural, or derivatized nucleotide bases. In some embodiments, a nucleic acid or polynucleotide can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

As used herein, the term “binding” refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶M, 10⁻⁷M, 10⁻⁸M, 10⁻⁹, 10⁻¹⁰M, 10⁻¹¹ M, 10⁻¹² M, 10⁻¹³M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd.

As used herein, the term “hybridizing” or “hybridize” refers to the pairing of substantially complementary or complementary nucleic acid sequences within two different molecules. Pairing can be achieved by any process in which a nucleic acid sequence joins with a substantially or fully complementary sequence through base pairing to form a hybridization complex. “Hybridizing” or “hybridize” can comprise denaturing the molecules to disrupt the intramolecular structure(s)(e.g., secondary structure(s)) in the molecule. In some embodiments, denaturing the molecules comprises heating a solution comprising the molecules to a temperature sufficient to disrupt the intramolecular structures of the molecules. In some instances, denaturing the molecules comprises adjusting the pH of a solution comprising the molecules to a pH sufficient to disrupt the intramolecular structures of the molecules. For purposes of hybridization, two nucleic acid sequences or segments of sequences are “substantially complementary” if at least 80% of their individual bases are complementary to one another. In some embodiments, a splint oligonucleotide sequence is not more than about 50% identical to one of the two polynucleotides (e.g., RNA fragments) to which it is designed to be complementary. The complementary portion of each sequence can be referred to herein as a ‘segment’, and the segments are substantially complementary if they have 80% or greater identity.

The terms “complementarity” and “complementary” mean that a nucleic acid can form hydrogen bond(s) with another nucleic acid based on traditional Watson-Crick base paring rule, that is, adenine (A) pairs with thymine (U) and guanine (G) pairs with cytosine (C). Complementarity can be perfect (e.g., complete complementarity) or imperfect (e.g., partial complementarity). Perfect or complete complementarity indicates that each and every nucleic acid base of one strand is capable of forming hydrogen bonds according to Watson-Crick canonical base pairing with a corresponding base in another, antiparallel nucleic acid sequence. Partial complementarity indicates that only a percentage of the contiguous residues of a nucleic acid sequence can form Watson-Crick base pairing with the same number of contiguous residues in another, antiparallel nucleic acid sequence. In some embodiments, the complementarity can be at least 70%, 80%, 90%, 100% or a number or a range between any two of these values. In some embodiments, the complementarity is perfect, i.e., 100%. For example, the complementary candidate sequence segment is perfectly complementary to the candidate sequence segment, whose sequence can be deducted from the candidate sequence segment using the Watson-Crick base pairing rules.

As used herein, the term “vector” refers to a polynucleotide construct, typically a plasmid or a virus, used to transmit genetic material to a host cell. Vectors can be, for example, viruses, plasmids, cosmids, or phage. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the terms “transfection” or “infection” refer to the introduction of a nucleic acid into a host cell, such as by contacting the cell with a recombinant MVA virus or a gutless picornaviral particle as described herein.

As used herein, the term “transgene” refers to any nucleotide or DNA sequence that is integrated into one or more chromosomes of a target cell by human intervention. In some embodiments, the transgene comprises a polynucleotide that encodes a protein of interest. The protein-encoding polynucleotide is generally operatively linked to other sequences that are useful for obtaining the desired expression of the gene of interest, such as transcriptional regulatory sequences. In some embodiments, the transgene can additionally comprise a nucleic acid or other molecule(s) that is used to mark the chromosome where it has integrated.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented.

As used herein, the terms “effective amount” or “pharmaceutically effective amount” or “therapeutically effective amount” refer to an amount sufficient to effect beneficial or desirable biological and/or clinical results.

The term “pharmaceutically acceptable excipient” as used herein refers to any suitable substance that provides a pharmaceutically acceptable carrier, additive or diluent for administration of a compound(s) of interest to a subject. Pharmaceutically acceptable excipient can encompass substances referred to as pharmaceutically acceptable diluents, pharmaceutically acceptable additives, and pharmaceutically acceptable carriers.

As used herein, a “subject” refers to an animal for whom a diagnosis, treatment, or therapy is desired. I some embodiments, the subject is a mammal. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs: dogs; cats; sheep; goats; cows: horses; primates, such as monkeys, chimpanzees and apes, and, in particular, humans. In some embodiments, the mammal is a primate. In some embodiments, the mammal is a human. In some embodiments, the mammal is not a human. In some aspects, the subject can have or is suspected of having a cardiovascular disease and/or has one or more symptoms of a cardiovascular disease. In some aspects, the subject is a human who is diagnosed with a risk of cardiovascular disease at the time of diagnosis or later. In some cases, the diagnosis with a risk of cardiovascular disease can be determined based on the presence of one or more mutations in an endogenous apolipoprotein (a) (LPA) gene or genomic sequence near the LPA gene in the genome that may affect the expression of the apo(a) protein.

The present disclosure provides effective compositions, methods, systems and means of delivery of CRISPR/Cas-mediated gene editing systems to the trabecular meshwork cells using lipid nanoparticles (LNP). As described herein, the compositions, methods, systems and means can be used for treating eye diseases, e.g., for treating myocilin-associated glaucoma.

Trabecular meshwork (TM) cells are the primary cell type that occupy and form the proximal portion of the conventional outflow pathway, the primary egress route for aqueous humor from the eye. TM cell has a pore-like structure, through which aqueous humor circulates to the canal of Schlemm. In the eye, myocilin gene (MYOC) is expressed in high amounts in the TM. Wild type myocilin is secreted into extracellular matrix (ECM) of TM, while the mutant form aggregates causing endoplasmic reticulum (ER) stress and death of TM cells. Some of the pathogenic potential of myocilin includes myocilin misfolding/unfolding; overexpression of myocilin; co-aggregation of Grp94, which limits autophagy; disruption of ECM homeostasis caused by mutant myocilin; oxidative stress (OS), ER stress and IL-1/NF-κB inflammatory stress caused by mutant myocilin; and instability resulting from conformational disorders caused by mutant myocilin.

Accordingly, provided herein are effective therapeutic approaches that delivery gene editing system to the TM cells of a subject's eyes (e.g., treating glaucoma patients with a mutation in the MYOC gene) which involve knocking down/reducing expression of a target gene or the protein encoded by the target gene (e.g., myocilin expression) in the TM cells. In these approaches, mutant and wild type myocilin alleles are targeted, TM cells are cleared of accumulated mutant myocilin to alleviate ER stress, and aqueous humor (AH) outflow is increased, and intraocular pressure (IOP) is decreased. Clinical readout includes measurement of IOP.

Methods and systems for treating a subject with glaucoma are provided herein in some embodiments, which target knocking down or knocking out myocilin (MYOC) gene in the subject's eye, more specifically, in the trabecular meshwork cells of the subject's eye.

### I. System for Delivering CRISPR/Cas-Mediated Gene Editing Systems to Trabecular Meshwork (TM)

In some aspects, provided herein are compositions, methods and systems for delivering a CRISPR/Cas-mediated gene editing system to TM cells in a subject. The compositions, methods and systems can be used, for example, for treating a subject with glaucoma. Such compositions, methods and systems can comprise (i) a gene editing means that targets reducing expression of a target gene (e.g., myocilin (MYOC) gene, ACTA2 gene) in the subject's eye; and (ii) a lipid nanoparticle (LNP) that delivers the gene editing means to the subject's eye.

The reduction of the expression of the target gene (e.g., MYOC gene, ACTA2 gene) may be achieved via gene editing (including genomic editing), a type of genetic engineering, in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out or knocked-down due to the sequence alteration.

Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

(a) Genetically Edited Genes

The system disclosed herein, once delivered to the TM cells of the subject's eye, causes a disrupted target gene (e.g., MYOC gene, ACTA2 gene). As used herein, a “disrupted gene” refers to a gene comprising an insertion, deletion or substitution relative to an endogenous gene such that expression of a functional protein from the endogenous gene is reduced or inhibited. As used herein, “disrupting a gene” refers to a method of inserting, deleting or substituting at least one nucleotide/nucleic acid in an endogenous gene such that expression of a functional protein from the endogenous gene is reduced or inhibited. Methods of disrupting a gene are known to those of skill in the art and described herein.

In some embodiments, a cell that comprises a disrupted gene does not express (e.g., at the cell surface) a detectable level (e.g., in an immune assay using an antibody binding to the encoded protein or by flow cytometry) of the protein encoded by the gene. A cell that does not express a detectable level of the protein may be referred to as a knockout cell. A cell that expresses a reduced level of the protein may be referred to as a knocked down cell.

(b) MYOC Gene Editing

Myocilin is a secreted 55-57 kDa glycoprotein that forms dimers and multimers. It has a myosin-like domain, a leucine zipper region and an olfactomedin domain. In the eye, myocilin is expressed in high amounts in the TM, sclera, ciliary body and iris, and at considerably lower amounts in retina and optic nerve head (Tamm, Prog Retin Eye Res. 2002 July; 21(4): 395-428).

In the present disclosure, the disrupted MYOC gene means that the expression of MYOC in the TM cells is substantially reduced/knocked down or eliminated completely. The disrupted MYOC gene may comprise one or more genetic edits at one or more suitable target sites (e.g., in coding regions or in non-coding regulatory regions such as promoter regions) that disrupt expression of the MYOC gene. Such target sites may be identified based on the gene editing approach. Exemplary target sites for the genetic edits may include exon 1, exon 2 or exon 3 of the MYOC gene, or a combination thereof. In some embodiments, one or more genetic editing may occur in exon 1.

Genetic editing can be induced by a gene editing technology, (e.g., the CRISPR/Cas technology) using an RNA-guided nuclease and a guide RNA targeting a site in the MYOC gene. In some embodiments, the RNA-guided nuclease may be a Cas9 nuclease, including but not limited to, a Staphylococcus aureus Cas9 (SaCas9) nuclease or a Streptococcus pyogenes Cas9 (SpCas9) nuclease.

In some embodiments, the MYOC site targeted by the guide RNA comprises any one of the nucleotide sequences listed in Table 1 or Table 2 (see, Sequence Tables below). In some embodiments, the site targeted by the guide RNA comprises a nucleotide sequence selected from the group consisting of SEQ ID NOs: 1-27 and 55-115. In some specific examples, the site targeted by the guide RNA comprises a nucleotide sequence selected from the group consisting of SEQ ID Nos: 64, 73, 74, 75, 76, and 115.

Exemplary MYOC-targeting guide RNAs can be deduced from the target sequences listed in Table 1 or Table 2, which are also within the scope of the present disclosure. In some embodiments, the guide RNA may be a SaCas9 sgRNA or SpCas9 sgRNA.

### II. Gene Editing Means

The reduction of the expression of the target gene (e.g., MYOC gene, ACTA2 gene) in the TM cells can be achieved via a conventional gene editing method or those described herein.

(a) Gene Editing Methods

Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

In some embodiments, gene disruption occurs by deletion of a genomic sequence using two guide RNAs. Methods of using CRISPR-Cas gene editing technology to create a genomic deletion in a cell (e.g., to knock out a gene in a cell) are known (Bauer D E et al. Vis. Exp. 2015; 95:e52118).

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration.

Some exemplary approaches are disclosed in detail below.

(b) CRISPR-Cas9 Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crispr RNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78). crRNA drives sequence recognition and specificity of the CRISPR-Cas9 complex through Watson-Crick base pairing typically with a 20 nucleotide (nt) sequence in the target DNA. Changing the sequence of the 5′ 20nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM). tracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB), where both strands of the DNA terminate in a base pair (a blunt end). After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells and occurs at a relatively low frequency in most cell types. In many embodiments of the present disclosure, NHEJ is utilized as the repair operant.

(c) Endonuclease for Use in CRISPR

In some embodiments, the Cas9 (CRISPR associated protein 9) endonuclease or a nucleotide sequence encoding the Cas9 endonuclease is used in a CRISPR system. The Cas9 enzyme may be one from Staphylococcus aureus (SaCas9), or one from Streptococcus pyogenes (SpCas9), although other Cas9 homologs may also be used. SaCas9 and SpCas9 DNA sequences are listed in Table 10 (see, Sequence Tables below). It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

In some embodiments, the CRISPR/Cas system comprises components derived from a Type-I, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types I, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9 and contains a RuvC-like nuclease domain.

In some embodiments, the Cas nuclease is from a Type-II CRISPR/Cas system (e.g., a Cas9 protein from a CRISPR/Cas9 system). In some embodiments, the Cas nuclease is from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

In some embodiments, a Cas nuclease comprises more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In some embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease).

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

(d) Guide RNAs (gRNAs)

The CRISPR technology involves the use of a genome-targeting nucleic acid that can direct the endonuclease to a specific target sequence within a target gene for gene editing at the specific target sequence. The genome-targeting nucleic acid can be an RNA. A genome-targeting RNA is referred to as a “guide RNA” or “gRNA” herein. A guide RNA comprises at least a spacer sequence that hybridizes to a target nucleic acid sequence within a target gene for editing, and a CRISPR repeat sequence.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

In some embodiments, the genome-targeting nucleic acid (e.g., gRNA) is a single-molecule guide RNA. A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

A spacer sequence in a gRNA is a sequence (e.g., a 20-nucleotide sequence) that defines the target sequence (e.g., a DNA target sequences, such as a genomic target sequence) of a target gene of interest. In some embodiments, the spacer sequence range from 15 to 30 nucleotides. For example, the spacer sequence may contain 15, 16, 17, 18, 19, 29, 21, 22, 23, 24, 25, 26, 27, 28, 29, or 30 nucleotides. In some embodiments, a spacer sequence contains 20 nucleotides.

The “target sequence” is in a target gene that is adjacent to a PAM sequence and is the sequence to be modified by an RNA-guided nuclease (e.g., Cas9). The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNGG-3′, the target nucleic acid can be the sequence that corresponds to the Ns, wherein N can be any nucleotide, and the underlined NGG sequence is the S. pyogenes PAM.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to I mismatch.

For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be an sgRNA, which may comprise a 20-nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence.

In some embodiments, the sgRNA comprises no uracil at the 3′ end of the sgRNA sequence. In some embodiments, the sgRNA comprises one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In some embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

In some embodiments, the gRNAs disclosed herein target a MYOC gene, for example, target a site within any one of exons 1-3 of MYOC, for example, exon 1, exon 2, or exon 3 of the MYOC gene. Such a gRNA may comprise a spacer sequence complementary (complete or partially) to the target sequences in exon 1 of a MYOC gene, or a fragment thereof. Exemplary target sequences of MYOC are provided in Tables 1-2 (see, Sequence Table below). Exemplary gRNA sequences can be deduced from the target sequences. In some embodiments, the gRNAs target a AC1A2 gene.

In some embodiments, the gRNA comprises a spacer sequence having a RNA sequence corresponding to any one of the target sequences set forth in SEQ ID NOs: 1-27 and SEQ ID NOs: 55-115 or a variant thereof. In some embodiments, the gRNA comprises a spacer sequence having a RNA sequence corresponding to any one of the target sequences set forth in SEQ ID NOs: 1-27 and SEQ ID NOs: 55-115. In some embodiments, the gRNA comprises a spacer sequence having one, two or three mismatches to a RNA sequence corresponding to any one of the target sequences set forth in SEQ ID NOs: 1-27 and SEQ ID NOs: 55-115. In some embodiments, the gRNA comprises a spacer sequence having about, at least, or at least about 80%, 85%, 9⁰% i, 95%, 97%, 98%, 99%, or 100% homology to a RNA sequence corresponding to any one of the target sequences set forth in SEQ ID NOs: 1-27 and SEQ ID NOs: 55-115.

A gRNA spacer sequence is the RNA equivalent of a target sequence. Accordingly, a gRNA sequence can comprise a spacer sequence corresponding to any one of SEQ ID NOs: 1-27 and SEQ ID NOs: 55-115 in which a “T” is substituted with a “U”. In some embodiments, the spacer sequence comprised in a gRNA sequence can be a variant of a spacer sequence having about, at least, or at least about 80%, 85%, 90%, 95%, 97%, 98%, 99%, or 100% homology to any spacer corresponding to any one of SEQ ID NOs: 1-27 and SEQ ID NOs: 55-115 in which a “T” is substituted with a “U”. In some embodiments, the spacer sequence comprised in a gRNA sequence can be a variant of a spacer sequence having one, two or three mismatches compared to any spacer corresponding to any one of SEQ ID NOs: 1-27 and SEQ ID NOs: 55-115 in which a “T” is substituted with a “U”.

In some embodiments, a gRNA comprises a spacer sequence having a RNA sequence corresponding to any one of the target sequences set forth in SEQ ID NOs: 6, 10, 15, 18 and 26 or a variant thereof. In some embodiments, the gRNA comprises a spacer sequence having one, two or three mismatches to a RNA sequence corresponding to any one of the target sequences set forth in SEQ ID NOs: SEQ ID NOs: 6, 10, 15, 18 and 26.

In some embodiments, a gRNA comprises a spacer sequence having a RNA sequence corresponding to any one of the target sequences set forth in SEQ ID NOs: 59, 61, 63, 64, 66, 69, 72-77, 79, 81, 82, 90, 95, 98-101, 104, 106, 107, 109, and 113-115 or a variant thereof. In some embodiments, the gRNA comprises a spacer sequence having one, two or three mismatches to a RNA sequence corresponding to any one of the target sequences set forth in SEQ ID NOs: 59, 61, 63, 64, 66, 69, 72-77, 79, 81, 82, 90, 95, 98-101, 104, 106, 107, 109, and 113-115. By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high-performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some embodiments, the gRNAs of the present disclosure are produced in vitro transcription (IVT), synthetic and/or chemical synthesis methods, or a combination thereof. Enzymatic (IVT), solid-phase, liquid-phase, combined synthetic methods, small region synthesis, and ligation methods are utilized. In some embodiments, the gRNAs are made using IVT enzymatic synthesis methods. Methods of making polynucleotides by IVT are known in the art and are described in WO2013/151666. Accordingly, the present disclosure also includes polynucleotides, e.g., DNA, constructs and vectors are used to in vitro transcribe a gRNA described herein.

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art. In some embodiments, non-natural modified nucleobases can be introduced into any of the gRNAs disclosed herein during synthesis or post-synthesis. In some embodiments, modifications are on internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

The chemically-modified gRNA can comprise one or more phosphorothioated 2′-O-methyl nucleotides at the 3′ end and/or the 5′ end of the gRNA. In some embodiments, the chemically-modified gRNA comprises phosphorothioated 2′-O-methyl nucleotides at the 3′ end of the gRNA. In some embodiments, the chemically-modified gRNA comprises phosphorothioated 2′-O-methyl nucleotides at the 5′ end of the gRNA. In some embodiments, the chemically-modified gRNA comprises three or four phosphorothioated 2′-O-methyl nucleotides at the 3′ end and/or three or four at the 5′ end of the gRNA. In some embodiments, any one of SEQ ID NOs: 18-25 and 26-31 can be chemically modified to have one, two three or four phosphorothioated 2′-O-methyl nucleotides at the 3′ end of the gRNA; one, two or three phosphorothioated 2′-O-methyl nucleotides at the 5′ end of the gRNA, or a combination thereof.

The number and position of the phosphorothioate linkages can vary. In some embodiments, the linkage can be between the first and second, the second and third, the third and fourth position, fourth and fifth, fifth and sixth, sixth and seventh, seventh and eighth, eighth and ninth, ninth or tenth, or further, position from the 5′ end of the gRNA. In some embodiments, the linkage can be between the first and second, the second and third, the third and fourth position, fourth and fifth, fifth and sixth, sixth and seventh, seventh and eighth, eighth and ninth, ninth or tenth, or further, position from the 3′ end of the gRNA.

In some embodiments, the nucleotide analogues/modifications can comprise 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, puromycin-5′-triphosphate, or xanthosine-5′-triphosphate. Base-modified nucleotides can comprise 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, i-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-i-methyl-pseudouridine, 1-methyl-I-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, I-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-I-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, or 7-deaza-adenosine.

At least one modified nucleotide and/or the at least one nucleotide analog can comprise 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl-2′-O-methyluridine, 5-carboxymethylaminomethyl-2-thiouridine, 5-(isopentenylaminomethyl)uridine, 5-(isopentenylaminomethyl)-2-thiouridine, or 5-(isopentenylaminomethyl)-2′-O-methyluridine.

In some embodiments, chemical modifications comprise pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine or 2′-O-methyluridine. In some embodiments, the modification comprises a 2′-O-methyluridine (2OMe-rU), a 2-O-methylcytidine (2′OMe-rC), 2′-O-methyladenosine (2′OMe-rA), or 2′-O-methylguanosine (2′OMe-rG).

In some embodiments, enzymatic or chemical ligation methods can be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments, a CRISPR/Cas nuclease system for use in genetically editing any of the target genes disclosed here includes at least one guide RNA. In some embodiments, the CRISPR/Cas nuclease system contains multiple gRNAs, for example, 2, 3, or 4 gRNAs. Such multiple gRNAs may target different sites in a same target gene. Alternatively, the multiple gRNAs may target different genes. In some embodiments, the guide RNA(s) and the Cas protein may form a ribonucleoprotein (RNP), e.g., a CRISPR/Cas complex. The guide RNA(s) may guide the Cas protein to a target sequence(s) on one or more target genes as those disclosed herein, where the Cas protein cleaves the target gene at the target site. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex.

In some embodiments, the indel frequency (editing frequency) of a particular CRISPR/Cas nuclease system, comprising one or more specific gRNAs, may be determined using a TIDE analysis, which can be used to identify highly efficient gRNA molecules for editing a target gene. In some embodiments, a highly efficient gRNA yields a gene editing frequency of higher than 80%. For example, a gRNA is considered to be highly efficient if it yields a gene editing frequency of at least 80%, at least 85%, at least 90%, at least 95%, or 100%.

(e) Other Gene Editing Methods

Besides the CRISPR method disclosed herein, additional gene editing methods as known in the art can also be used. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121; 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and WO/SPBc/TP901-1, whether used individually or in combination.

Any of the nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells. Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids. Some specific examples are provided below.

### III. Delivery of Guide RNAs and Nucleases to TM Cells

The CRISPR/Cas nuclease system disclosed herein, comprising a guide RNA (gRNA) or a nucleic acid sequence encoding the gRNA and an RNA-guided nuclease or a nucleic acid sequence encoding the RNA-guided endonuclease, can be delivered to a TM cell for genetic editing of a target gene (e.g., the MYOC gene or ACTA2 gene), via a conventional method. In some embodiments, components of a CRISPR/Cas nuclease system as disclosed herein may be delivered to a target cell separately, either simultaneously or sequentially. In other embodiments, the components of the CRISPR/Cas nuclease system may be delivered into a target together, for example, as a complex. In some instances, a gRNA and an RNA-guided nuclease can be pre-complexed together to form a ribonucleoprotein (RNP), which can be delivered into a target cell.

RNPs are useful for gene editing, at least because they minimize the risk of promiscuous interactions in a nucleic acid-rich cellular environment and protect the RNA from degradation. Methods for forming RNPs are known in the art. In some embodiments, an RNP containing an RNA-guided nuclease (e.g., a Cas nuclease, such as a Cas9 nuclease) and a guide RNA targeting the MYOC gene can be delivered to a TM cell. In some embodiments, an RNP can be delivered to a TM cell by electroporation.

In some embodiments, an RNA-guided nuclease can be delivered to a cell in a DNA vector that expresses the RNA-guided nuclease in the cell. In other examples, an RNA-guided nuclease can be delivered to a cell in an RNA that encodes the RNA-guided nuclease and expresses the nuclease in the cell. Alternatively or in addition, a gRNA targeting a gene can be delivered to a cell as a RNA, or a DNA vector that expresses the gRNA in the cell.

Delivery of an RNA-guided nuclease, gRNA, and/or an RNP may be through direct injection or cell transfection using known methods, for example, electroporation or chemical transfection. Other cell transfection methods may be used.

In some embodiments, the one or more of the nucleic acid sequences and/or polypeptides can be delivered to cells, either in vitro or in vivo, via viral based or non-viral based delivery systems, including adenovirus vectors, adeno-associated virus (AAV) vectors, retrovirus vectors, lentiviral vectors, herpes virus vectors, nanoparticles, liposomes, lipid nanoparticles, poxviruses, naked DNA administration, plasmids, cosmids, phages, encapsulated cell technology, and the like.

In some embodiments, the gRNA and a RNA-guided endonuclease or a nucleic acid encoding the RNA-guided endonuclease or a composition thereof can be formulated in a liposome or lipid nanoparticle. In some embodiments, the RNA-guided nuclease and the guide RNA can be delivered to the trabecular meshwork cells by a lipid nanoparticle (LNP). The term “lipid nanoparticle” refers to a nanoscopic particle composed of lipids having a size measured in nanometers (e.g., 1-5,000 nm). Size of the LNP in the LNP formulations described herein (e.g., CTX-C12-CT LNP formulation) can vary. In some embodiments, the LNPs have a mean diameter of about, at least, at least about, at most or at most about 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm or a number or a range between any of these values. In some embodiments, the lipid nanoparticle particle size is about 50 to about 200 nm in diameter, or about 70 to about 180 nm in diameter, or about 80 to about 150 nm in diameter. In some embodiments, the particle size (e.g., mean diameter) of the LNP is in the 85-95 nm range. In some embodiments, the particle size (e.g., mean diameter) of the LNP is about 190 nm, 195 nm, 200 nm, 205 nm, or a range between any two of these values. Without being bound by any particular theory, it is believed that it can be advantageous to use small size LNP to deliver payload to the trabecular meshwork. For example, it can be advantageous to use LNP with the size of 75 nm, 80 nm, 85 nm, 90 nm, 95 nm, 100 nm, 105 nm, or a number or a range between any two of these values to deliver a CRISPR/Cas-mediated gene editing system to the trabecular meshwork cells of a subject.

In some embodiments, the lipids comprised in the lipid nanoparticles comprise cationic lipids and/or ionizable lipids. Any suitable cationic lipids and/or ionizable lipids known in the art can be used to formulate LNPs for delivery of gRNA and Cas endonuclease to the cells. Exemplary cationic lipids include one or more amine group(s) bearing positive charge. The lipid nanoparticles can further comprise one or more neutral lipids, charged lipids, sterols, tocopherols, hopanoids and polymers conjugated lipids such as poly(ethylene glycol) (PEG)-lipid.

The LNPs described herein can comprise one or more ionizable cationic lipid described herein. For example, the LNP can comprise one or more ionizable cationic lipids selected from the group consisting of: C12-200, cKK-E12, DLIN-MC3, DLIN-MC4, DLIN-MC5, DODMA, or DOTAP. The ionizable cationic lipid can be from about 30 mol % to about 70 mol % (e.g 30 mol %, 35 mol %, 40 mol %, 45 mol %, 50 mol %, 55 mol %, 60 mol %, 65 mol %, 70 mol %) of the total lipid present in the LNP. As used herein, “mol percent” refers to a component's molar percentage relative to total mols of all lipid components in the LNP (i.e., total mols of cationic lipids, neutral lipids, sterol and polymer conjugated lipids). In some embodiments, the LNP include from about 40% to about 60% ionizable cationic lipid of the total lipid in the LNP. For instance, the lipid nanoparticles can include about 40%, 45%, 50% or 60% ionizable cationic lipid of the total lipid on a molar basis (based upon 100% total moles of lipids in the LNP). In some embodiments, the LNP comprises about 50 mol percent ionizable cationic lipids described herein.

The LNPs described herein can further comprise one or more non-cationic lipids (helper lipids). In some embodiments, the LNP can further comprise one or more neutral lipids, charged lipids, sterols, and polymers conjugated lipids. In some embodiments, the lipid nanoparticle comprises one or more neutral or zwitterionic lipids. The term “neutral lipid” refers to any of a number of lipid species which exist either in an uncharged or neutral zwitterionic form at physiological pH. The selection of neutral lipids and other non-cationic lipids for use in the particles described herein is generally guided by consideration of, for example, lipid particle size and stability of the lipid particle in the bloodstream. In some embodiments, the non-cationic lipids contain saturated fatty acids with carbon chain lengths in the range of C₁₀ to C₂₀. In some embodiments, non-cationic lipids with mono- or di-unsaturated fatty acids with carbon chain lengths in the range of C₁₀ to C₂₀ are used. Additionally, non-cationic lipids having mixtures of saturated and unsaturated fatty acid chains can be used. Suitable neutral lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-I-carboxylate (DOPE-mal), dipalmitoyl phosphatidyl ethanolamine (DPPE), dimyristoylphosphoethanolamine (DMPE), dimyristoyl phosphatidylcholine (DMPC), distearoyl-phosphatidyl-ethanolamine (DSPE), SM, 16-O-monomethyl PE, 16-O-dimethyl PE, 18-1-trans PE, 1-stearoyl-2-oleoyl-phosphatidyethanolamine (SOPE), cholesterol, or a mixture thereof. In some embodiments, the helper lipid is, or comprises, a PC class lipid (e.g., DLPC (12:0), DMPC (14:0), DPPC (16:0), DSPC (18:0), DOPC (18:1), DUPC (18:2), POPC (16:0, 18:1), SOPC (18:0, 18:1)); a PE class like lipid (e.g., DOPE (18:1), DSPE (18:0), DPPE (16:0), DMPE (14:0) SOPE (18:0, 18:1), POPE (16:0, 18:1)); a PG class like lipid (e.g., DOPG (18:1), DPPG (16:0)), or a mixture thereof. In some embodiments, the helper lipid is, or comprise, 1,2-dilauroyl-sn-glycero-3-phosphocholine (DLPC), DMPC, DPPC, DSPC, DOPC, diundecanoylphosphatidylcholine (DUPC), POPC, 1-Stearoyl-2-oleoyl-sn-glycero-3-phosphocholine (SOPC), DOPE, DSPE, DPPE, DMPE, 1-stearoyl-2-oleoyl-sn-glycero-3-phosphoethanolamine (SOPE, 18:0-18:1 PE), POPE, Dioleoyl phosphatidylglycerol (DOPG), Dipalmitoyl-sn-glycero-3-PG (DPPG), or a mixture thereof.

In some embodiments, the neutral lipids can be from about 5 mol % to about 20 mol % (e.g., about 5 mol %, 10 mol %, 15 mol %, 20 mol %) of the total lipid present in the LNP. In some embodiments, the LNP include from about 10% neutral lipid of the total lipid in the LNP on a molar basis (based upon 100% total moles of lipids in the LNP).

The LNP can further comprise a sterol, such as cholesterol. The sterol can be about 10 mol % to about 60 mol %, optionally about 20 mol % to about 50 mol %, more optionally about 30% to about 40% of the total lipid present in the LNP. In some embodiments, the sterol is about 20 mol %, 25 mol %, 30 mol %, 35 mol %, 40 mol %, 45 mol %, or 50 mol % of the total lipid present in the LNP. In the LNP described herein, the sterol can be one or more of cholesterol, sitosterol, campesterol, plant sterols (also called phytosterols, e.g., stigmasterol, p-sitosterol), sterols from algae (e.g., fucosterol), sterols from animals (also called “zoosterols”), and sterols from fungi and protozoa (e.g., ergosterol). The LNPs disclosed herein can comprise tocopherols and hopanoids (Diploptene and Diplopterol) classes of compounds. In some embodiments, tocopherols and hopanoids (Diploptene and Diplopterol) classes of compounds are for replacing the sterols in the LNPs. In some embodiments, tocopherols and hopanoids (Diploptene and Diplopterol) classes of compounds are present in the LNP in addition to the sterol.

The LNP can further comprise polymer conjugated lipids such as polyethylene glycol (PEG)-modified lipids. Exemplary PEG-conjugated lipid include, for example, a PEG-diacylglycerol (DAG), a PEG-dialkyloxypropyl (DAA), a PEG-phospholipid, a PEG-ceramide (Cer), a PEG-dimyristoyl glycerol (DMG), or a mixture thereof. In some embodiments, the PEG conjugated lipid can be about 0 mol % to about 10 mol % of the total lipid in the LNP. For example, the PEG conjugated lipid is about 0 mol %, 0.5 mol %, 1 mol %, 1.5 mol %, 2 mol %, 3 mol %, 4 mol %, 5 mol %, 6 mol %, 7 mol %, 8 mol %, 9 mol % or 10 mol % ofthe total lipid present in the LNP. In some embodiments, the polymer conjugated lipid (e.g., PEG conjugated lipid) is about 0 mol % to about 5 mol % (e.g., 0.5%, 1%, 1.5%, 2%, 2.5%, 3%) of the total lipid present in the LNP. The PEG-modified lipid can be or can comprise, for example, DMG-PEG, DSG-PEG, a PEG-ceramide, a PEG-phospholipid, or a combination thereof.

In some embodiments of the LNP described herein, the ionizable cationic lipid may be C12-200, cKK-E12, DLIN-MC3, DLIN-MC4, DLIN-MC5, DODMA, or DOTAP; the helper lipid may be 1,2-distearoyl-sn-glycero-3-phosphocholine (DSPC) or 1,2-Dioleoyl-sn-glycero-3-phosphocholine (DOPC): the sterol may be cholesterol or sitosterol; and the PEG-lipid may be DMG-PEG.

In some embodiments, the LNP comprises about 50 mol % of C12-200, DLIN-MC3, DLIN-MC4, DLIN-MC5, DODMA and/or DOTAP, about 10 mol % of DSPC, about 37.0-39.5 mol % of cholesterol or sitosterol, and about 0.5-3.0% of DMG-PEG. In some specific examples, the LNP comprises about 50 mol % of C12-200, about 10 mol % of DSPC, about 37.0-39.5 mol % of sitosterol, and about 0.5-1.5% of DMG-PEG.

The LNP can be administered by any appropriate route that results in delivery to the patient's eye. For example, an effective amount of the LNP may be administered to the patient by intravitreal injection and/or intracameral injection.

### IV. Compositions and Therapeutic Applications

The Cas9 gRNA system disclosed herein can be administered to a subject for therapeutic purposes, for example, treatment of a myocilin-associated glaucoma. Disruption of the MYOC gene knocks down/reduces myocilin expression in the trabecular meshwork (TM) cells, which in turn leads to clearance of accumulated mutant myocilin thereby alleviating ER stress, increasing aqueous humor (AH) outflow, and decreasing intraocular pressure (IOP).

Accordingly, provided herein is a method for treating a subject with a glaucoma, the method comprising reducing expression of myocilin (MYOC) gene in the TM cells of the subject's eye. In some embodiments, the method for treating a subject with a glaucoma comprises administering to the subject a plurality of lipid nanoparticles (LNPs) complexed with (a) a guide RNA targeting myocilin (MYOC) gene or a nucleic acid encoding the guide RNA; and (b) a RNA-guided endonuclease or a nucleic acid encoding the RNA-guided endonuclease, thereby reducing expression of MYOC gene in the subject's eye. In some embodiments, the glaucoma may be myocilin-associated glaucoma. In some embodiments, the glaucoma may be POAG. In some embodiments, the expression of the MYOC gene may be reduced in the TM cells of the subject's eye.

As described above and herein, the MYOC gene may be disrupted in the TM cells by CRISPR/Cas-mediated gene editing system comprising an RNA-guided nuclease (or a nucleotide sequence encoding the RNA-guided nuclease) and a guide RNA targeting a site in the AMYOC gene (e.g., exon 1, 2 or 3 of MYOC gene). In some embodiments, the CRISPR/Cas-mediated gene editing system can be provided in a pharmaceutical composition. Accordingly, a composition can include one or more gRNA(s) (MYOC gRNA), a RNA-guided endonuclease or a nucleotide sequence encoding the RNA-guided endonuclease described herein. The MYOC gRNAs can be any gRNA described herein targeting one or more target sequences of SEQ ID NOs: 1-27 and 55-115 or a variant thereof. The RNA-guided endonuclease or a nucleotide sequence encoding the RNA-guided endonuclease can be any RNA-guided endonuclease described herein. In some embodiments, the DNA endonuclease is Cas9. In some embodiments, the Cas9 endonuclease is Streptococcus pyogenes (SpCas9) nuclease. In some embodiments, the Cas9 endonuclease is Staphylococcus aureus Cas9 (SaCas9) nuclease.

In some embodiments, the RNA-guided nuclease and the guide RNA are delivered to the trabecular meshwork cells by a lipid nanoparticle (LNP). The LNP can comprise one or more ionizable cationic lipid selected from (C12-200, cKK-E12, DLIN-MC3, DLIN-MC4, DLIN-MC5, DODMA, or DOTAP; a helper lipid of 1,2-distearoyl-sn-glycero-3-phosphocholine (DSPC) and/or 1,2-Dioleoyl-sn-glycero-3-phosphocholine (DOPC); a sterol of cholesterol and/or sitosterol; and the PEG-lipid of DMG-PEG. The LNP can comprise one or more PEG glycerides, e.g., the PEG glycerides in DMG-PEG and DSG-PEG classes, In some embodiments, the LNP comprises one or more DSG-PEG. The LNP can comprise one or more the PEG-Ceramide such as C16-PEG 2000 Ceramide or C8 PEG2000 Ceramide; one or more of PEG phospholipids, such as 14:0 PEG 2000 PE; or any combination thereof. In some embodiments, the LNP comprise a PEG-Ceramide. In some embodiments, the LNP comprise a PEG-phospholipid.

In some embodiments, the LNP comprises about 50 mol % of C12-200, DLIN-MC3, DLIN-MC4, DLIN-MC5, DODMA and/or DOTAP, about 10 mol % of DSPC, about 37.0-39.5 mol % of cholesterol or sitosterol, and about 0.5-3.0% of DMG-PEG. In some specific examples, the LNP comprises about 50 mol % of C12-200, about 10 mol % of DSPC, about 37.0-39.5 mol % of sitosterol, and about 0.5-1.5% of DMG-PEG.

In some embodiments, the compounds of the composition described herein are encapsulated in the lipid portion of the lipid nanoparticle or an aqueous space enveloped by some or all of the lipid portion of the lipid nanoparticle. The encapsulation can be full encapsulation, partial encapsulation, or both. In some embodiments, the nucleic acid and/or polypeptides are fully encapsulated in the lipid nanoparticle.

In some embodiments, one or more compounds herein described are associated with a liposome or lipid nanoparticle via a covalent bond or non-covalent bond. In some embodiments, any of the compounds in the composition can be separately or together contained in a liposome or lipid nanoparticle.

The LNP can be administered by any appropriate route that results in delivery to the patient's eye. For example, an effective amount of the LNP may be administered to the patient by intravitreal injection and/or intracameral injection.

An effective amount refers to the amount of LNP needed to achieve levels of editing to prevent or alleviate at least one or more signs or symptoms of a medical condition, and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment using the means disclosed herein can be determined by the skilled clinician. A treatment is considered “effective”, if any one or all of the signs or symptoms of, as but one example, levels of functional target are altered in a beneficial manner (e.g., increased by at least 10%), or other clinically accepted symptoms or markers of disease (e.g., glaucoma) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments, the plurality of LNPs is administered to the subject (e.g., locally) at a dose of about 0.1-5 mg/kg (determined by the total nucleic acids (e.g., the total of the target gene gRNA (e.g., MYOC gRNA) and Cas9 mRNA)) per administration, including 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, 4.5 mg/kg, or 5 mg/kg, or a number or a range between any two of these values. In some embodiments, the plurality of LNP is administered to the subject at a dose of, or a dose about, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 2 mg/kg, 2.5 mg/kg or 3.0 mg/kg (in some embodiments, determined by the total of the target gene gRNA (e.g., MYOC gRNA) and Cas9 mRNA). In some embodiments, the plurality of LNPs is administered at a dose of about 40 ug/eye, 45 ug/eye, 50 ug/eye, 55 ug/eye, 60 ug/eye, 65 ug/eye, 70 ug/eye, 75 ug/eye, 80 ug/eye, 85 ug/eye, 90 ug/eye, 95 ug/eye, 100 ug/eye, or a number or a range between any two of these values.

In some embodiments, a composition described above can further have one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. In some embodiments, a composition can also include one or more components that can be used to facilitate or enhance the on-target binding or the cleavage of DNA by the endonuclease, or improve the specificity of targeting.

In some embodiments, any components of a composition are formulated with pharmaceutically acceptable excipients such as carriers, solvents, stabilizers, adjuvants, diluents, etc., depending upon the particular mode of administration and dosage form. In embodiments, guide RNA compositions are generally formulated to achieve a physiologically compatible pH, and range from a pH of about 3 to a pH of about 11, about pH 3 to about pH 7, depending on the formulation and route of administration. In some embodiments, the pH is adjusted to a range from about pH 5 to about pH 8.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition will depend on the nature of the disorder or condition, and can be determined by standard clinical techniques.

In some embodiments, the compounds herein described (e.g., a RNA-guided endonuclease or a nucleic acid encoding the RNA-guided endonuclease and/or gRNA) of a composition can be delivered via transfection such as calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. In some embodiments, the composition is introduced to the cells via lipid-mediated transfection using a lipid nanoparticle.

The compositions herein described can be administered to a subject in need thereof to treat an eye disease (e.g., glaucoma). Accordingly, the present disclosure also provides a gene therapy approach for treating a glaucoma in a patient by reducing expression of MYOC gene (e.g., a wildtype MYOC gene or a mutant MYOC gene) in the TM cells of the subject's eye. In some embodiments, a method for treating a subject with a type of glaucoma is disclosed. The method comprises reducing expression of myocilin (MYOC) gene in the subject's eye. In some embodiments, the method comprises administering to a subject a plurality of LNPs complexed with (a) a guide RNA (gRNA) or a nucleic acid encoding a gRNA that targets MYOC gene, and (b) a RNA-guided endonuclease or a nucleic acid encoding the RNA-guided endonuclease, thereby relieve the glaucoma.

The subject can be administered with the plurality of nanoparticles one time. In some embodiments, the subject can be administered with the plurality of nanoparticles two or more times, for example twice, for the treatment. Two administrations of the nanoparticles to the subject can be separated by a suitable time period. In some embodiments, the suitable time period is, or is about, one week, two weeks, three weeks, four weeks, five weeks, six weeks, seven weeks, eight weeks, three months, four months, five months, six months, a year, two years, three years, or more. In some embodiments, two of the two or more administrations are about two weeks to about two months apart, for example about three weeks. In some embodiments, each two of the two or more administrations are about two weeks to about two months apart, for example about three weeks. In some embodiments, two of the two or more administrations are about one month to about four months apart, for example about two months or three months, or longer. In some embodiments, each two of the two or more administrations are about one month to about four months apart, for example about two months or three months. In some embodiments, two of the two or more administrations are at least two months or three months apart. In some embodiments, each two of the two or more administrations are at least two months or three months apart. In some embodiments, the expression level of the target gene (e.g., MYOC gene) in the subject (e.g., in the subject's eye) receiving a single administration of the composition herein described can be substantially reduced (e.g., by at least 20%, 30%, 35%, 40%, 45%, 50%, 55%, 60%, 65%, 70%, 75%, 80%, 85%, 90°/%, 91%, 92%, 93%, 94%, 95%, 96%, 97%, 98%, 99% or higher) and remains at the reduced level for at least two months, three months, four months, six months, ten months, one year, eighteen months, two years, three years, four years, five years, ten years, fifteen years, twenty years, or longer after the administration. In some embodiments, the level of the protein encoded by the target gene (e.g., myocilin protein level) in the subject (e.g., in the subject's eye) receiving a single administration of the composition herein described can be substantially reduced (e.g., by at least 20%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or higher) and remains at the reduced level for at least two months, three months, four months, six months, ten months, one year, eighteen months, two years, three years, four years, five years, ten years, fifteen years, twenty years, or longer after the administration. The suitable time period between two administrations can be the same as or different from the suitable time period between another two administrations.

In some embodiments, the target tissue for the compositions and methods described herein is trabecular meshwork tissue. In some embodiments, the target cells for the compositions and methods described herein is trabecular meshwork cells.

In some embodiments, the pharmaceutical composition thereof can be administered by any suitable routine that can deliver the compounds to the target tissue/cells. For example, the pharmaceutical composition can be delivered via intravitreal, intracameral, subconjunctival, subtenon, retrobulbar, topical, suprachoroidal and/or posterior juxtascleral administration. In some embodiments, the pharmaceutical composition is administered to the subject by intravitreal injection or intracameral injection. The administration can be local. In some embodiments, more than one administration can be employed to achieve the desired level of gene expression over a period of various intervals, e.g., daily, weekly, monthly, or yearly.

The subject in need thereof can have myocilin-associated glaucoma. Myocilin-associated glaucoma includes the types of glaucoma linked with alterations in the myocilin gene (MYOC). In some embodiments, the myocilin-associated glaucoma includes open-angle glaucoma (OAG). In some embodiments, the OAG is primary OAG (POAG). In some embodiments, the OAG is juvenile-onset OAG (JOAG).

In some embodiments, following the administration the expression of the target gene (e.g., MYOC) in the subject's eye is reduced by at least 20%, by at least 30%, by at least 40%, by at least 500, by at least 60%, by at least 70%, by at least 80%, or by at least 900. In some embodiments, following the administration the expression of the protein encoded by the target protein (e.g., myocilin protein, α-SMA protein) in the trabecular meshwork cells of the subject's eye is reduced by at least 20%, by at least 30%, by at least 40%, by at least 50%, by at least 60%, by at least 70%, by at least 80%, or by at least 90%. The reduction is relative to the myocilin expression or the concentration of myocilin protein the trabecular meshwork cells of the subject (e.g., a mammal, a NHP, a human subject) prior to being administered with the plurality nanoparticles. In some embodiments, the reduction is relative to the expression of the target gene or the protein encoded by the target gene (e.g., MYOC expression or the concentration of myocilin protein) in one or more untreated subjects. In some embodiments, the reduction is relative to a reference level of the expression of the target gene (e.g., MYOC expression) or the concentration of the protein encoded by the target gene (e.g., myocilin protein) of healthy and/or unmodified subjects.

In some embodiments, the method can further comprise measuring intraocular pressure in the subject prior to, during, and/or after the administration. In some embodiments, the method comprises identifying a subject in need of the treatment. In some embodiments, a subject in need can be identified as having an elevated intraocular pressure (IOP). The compositions and methods described herein can reduce the IOP in a subject by at least at least 20%, by at least 40%, by at least 70%, or by at least 90% after the administration.

Combination therapies are also encompassed by the present disclosure. For example, the means disclosed herein may be co-used with other therapeutic agents, for treating the same indication, or for enhancing efficacy of MYOC gene editing in the TM cells and/or reducing side effects of the MYOC gene editing in the TM cells.

### V. Kits

The present disclosure also provides kits for therapeutic uses. In some embodiments, a kit provided herein may comprise components for performing genetic edit of a MYOC gene in the TM cells. The components for genetically editing the MYOC gene may comprise a suitable endonuclease such as an RNA-guided endonuclease and a nucleic acid guide, which direct cleavage of one or more suitable genomic sites by the endonuclease. For example, the kit may comprise a mRNA encoding Cas enzyme such as Cas 9 and one or more gRNAs targeting MYOC. Any of the gRNAs specific to the AYOC gene can be included in the kit.

Any of the kit disclosed herein may further comprise instructions of use. In some embodiments, the included instructions comprises a description of using the gene editing components to genetically edit the MYOC gene.

In some embodiments, a kit as disclosed herein may comprise instructions for administration of the LNPs as disclosed herein to achieve the intended therapeutic effect. Alternatively or in addition, the kit may further comprise a description of selecting a subject suitable for treatment based on identifying whether the subject is in need of the treatment. The instructions relating to the use generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the contents/components of the kit are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an infusion device for administration of the contents. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiments, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture. Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985; Transcription and Translation (B. D. Hames & S. J. Higgins, eds. (1984)); Animal Cell Culture (R. I. Freshney, ed. (1986)); Immobilized Cells and Enzymes (IRL Press, (1986)); and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: In Vitro ssRNA Screening

This example describes screening of guide RNAs targeting WIOC by CRISPR/Cas-mediated gene editing.

HEK293T cells were used for in vitro sgRNA screening and nucleofected with Cas9 (SaCas9 or SpCas9) and gRNA ribonucleoprotein complexes. The genomic DNA was extracted, and specific DNA sequences were PCR amplified and subjected to tracking of indels by decomposition (TIDE) analyses. Total and frameshift insertion+ deletion (INDEL) % were both reported in Table 6 (for SaCas9) and Table 7 (for SpCas9). Frameshift INDEL % refers to INDELs expected to result in a frameshift mutation in the MYOC coding sequence (i.e., ±I nt, f 2 nt, ±4 nt). Thus, Frameshift INDEL % refers to frequency of total INDELs minus frequency of INDELs that are f 3 nt, ±6 nt, ±9 nt, etc.

TABLE 6
Quantification of Editing Efficiency of SaCas9
sgRNAs Targeting MYOC Coding Sequence

| | | |
|---|---|---|
| sgRNA | Indels (%) | Indels (%) |

| name | Total | +/−SEM | Frameshift | +/−SEM |
|---|---|---|---|---|
| SaMCh1 | 2.09 | 0.79 | 1.40 | 0.37 |
| SaMCh2 | 23.70 | 2.08 | 23.14 | 2.18 |
| SaMCh3 | 45.20 | 8.44 | 18.59 | 5.27 |
| SaMCh4 | 1.48 | 0.66 | 0.93 | 0.34 |
| SaMCh6 | 58.13 | 4.50 | 54.50 | 4.82 |
| SaMCh7 | 14.26 | 2.01 | 10.73 | 1.47 |
| SaMCh8 | 33.69 | 2.74 | 29.53 | 2.23 |
| SaMCh9 | 11.48 | 1.36 | 5.65 | 0.81 |
| SaMCh10 | 89.65 | 2.46 | 80.83 | 2.07 |
| SaMCh11 | 15.08 | 2.45 | 8.43 | 2.06 |
| SaMCh12 | 9.46 | 1.84 | 8.70 | 1.79 |
| SaMCh13 | 22.59 | 1.65 | 19.58 | 1.62 |
| SaMCh14 | 21.37 | 3.30 | 20.87 | 3.15 |
| SaMCh15 | 52.87 | 8.11 | 48.63 | 9.03 |
| SaMCh16 | 21.18 | 2.71 | 10.96 | 1.70 |
| SaMCh17 | 3.59 | 2.13 | 2.74 | 1.80 |
| SaMCh18 | 50.81 | 4.29 | 47.43 | 4.02 |
| SaMCh19 | 35.35 | 6.13 | 34.61 | 5.57 |
| SaMCh20 | 7.21 | 1.25 | 3.24 | 0.54 |
| SaMCh21 | 2.48 | 0.59 | 1.20 | 0.30 |
| SaMCh22 | 7.74 | 0.60 | 6.33 | 0.80 |
| SaMCh23 | 15.86 | 4.22 | 12.89 | 3.49 |
| SaMCh24 | 29.43 | 7.74 | 27.93 | 8.19 |
| SaMCh25 | 12.74 | 8.57 | 11.64 | 8.96 |
| SaMCh26 | 57.83 | 10.57 | 57.13 | 10.36 |
| SaMCh27 | 3.46 | 0.81 | 2.81 | 0.70 |

* Frameshift INDEL % refers to INDELs expected to result in a frameshift mutation in the FAAH coding sequence (i.e., ±1 nt, ±2 nt, ±4 nt). Thus, Frameshift INDEL % refers to frequency of total INDELs minus frequency of INDELs that are ±3 nt, ±6 nt, ±9 nt, etc. All data entered are a mean of n = 3

replicates.

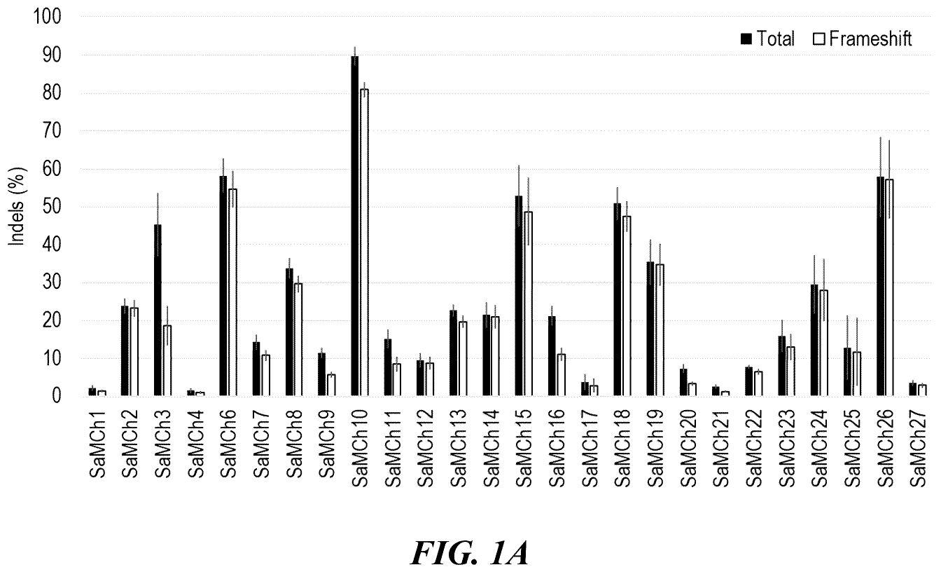

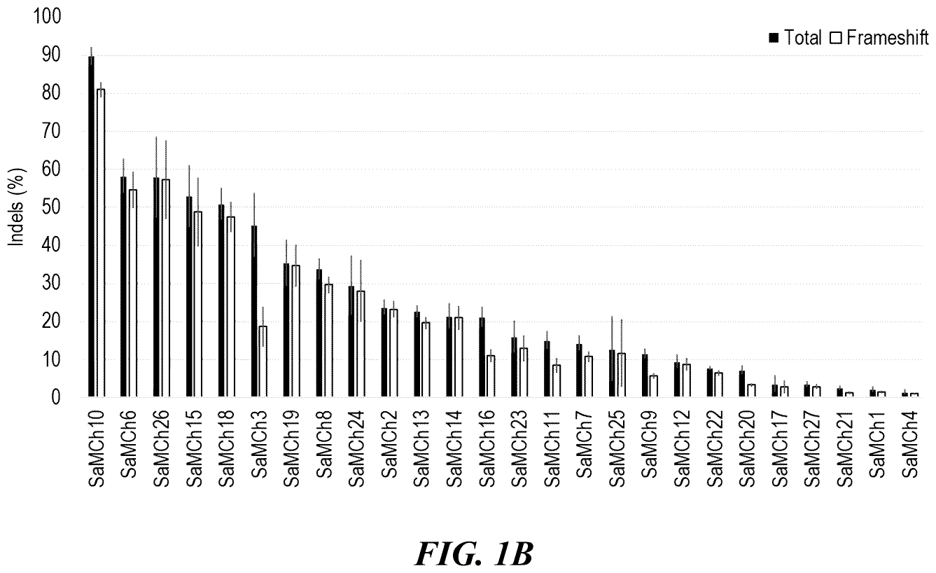

The results of quantification of editing efficiency of SaCas9 sgRNAs targeting MYOC coding sequence are shown in FIGS. 1A-1B. FIG. 1A illustrates the quantification ranked by guide numbering, and FIG. 1B illustrates the quantification ranked by INDEL percentages. As shown therein, ribonucleoprotein complexes of SaCas9 protein and one of 27 SaCas9 sgRNAs were transfected in IEK293T cells and cells harvested at a later time point. Following DNA extraction, PCR and TIDE were performed to determined percentage of indels at cut site. Total indel percentage ranged from 89.65% for SaMCh10 to 1.48% for SaMCh4 in conditions tested. The highest frameshift percentage (80.83%) was also seen in SaMCh 10 sgRNA transfected sample.

TABLE 7
Quantification of Editing Efficiency of SpCas9
sgRNAs Targeting MYOC Coding Sequence

| sgRNA | Indels (%) | | Indels (%) | |
|---|---|---|---|---|
| name | Total | +/−SEM | Frameshift | +/−SEM |
| SpMCh1 | 47.11 | 10.51 | 46.31 | 10.01 |
| SpMCh2 | 30.35 | 1.77 | 16.31 | 1.75 |
| SpMCh3 | 3.06 | 0.68 | 1.91 | 0.51 |
| SpMCh4 | 47.55 | 2.65 | 31.25 | 1.64 |
| SpMCh5 | 73.15 | 2.37 | 70.96 | 2.25 |
| SpMCh 6 | 50.06 | 6.44 | 43.53 | 7.14 |
| SpMCh 7 | 80.73 | 2.15 | 78.43 | 2.11 |
| SpMCh8 | 24.83 | 0.51 | 24.30 | 0.53 |
| SpMCh9 | 54.63 | 1.70 | 45.06 | 1.70 |
| SpMCh10 | 85.24 | 2.33 | 84.23 | 2.16 |
| SpMCh11 | 6.05 | 0.78 | 1.94 | 0.36 |
| SpMCh12 | 59.60 | 2.24 | 51.09 | 1.33 |
| SpMCh13 | 9.56 | 2.20 | 8.31 | 2.28 |
| SpMCh14 | 22.08 | 1.85 | 12.49 | 1.86 |
| SpMCh15 | 67.61 | 1.68 | 49.90 | 0.64 |
| SpMCh16 | 26.69 | 2.69 | 21.86 | 2.25 |
| SpMCh17 | 4.34 | 0.45 | 1.94 | 0.27 |
| SpMCh18 | 66.83 | 2.24 | 60.99 | 2.23 |
| SpMCh19 | 63.60 | 2.06 | 61.25 | 2.23 |
| SpMCh20 | 76.53 | 6.69 | 71.35 | 7.23 |
| SpMCh21 | 87.25 | 1.97 | 78.53 | 1.74 |
| SpMCh22 | 69.20 | 4.52 | 63.65 | 3.35 |
| SpMCh23 | 63.98 | 11.06 | 51.65 | 10.58 |
| SpMCh24 | 39.13 | 3.86 | 33.85 | 3.23 |
| SpMCh25 | 70.33 | 3.83 | 59.08 | 4.03 |
| SpMCh26 | 29.38 | 16.13 | 23.89 | 13.17 |
| SpMCh27 | 88.00 | 3.04 | 80.66 | 3.02 |
| SpMCh28 | 78.93 | 1.12 | 72.70 | 1.26 |
| SpMCh29 | 12.15 | 4.87 | 7.91 | 2.93 |
| SpMCh30 | 28.30 | 5.33 | 7.01 | 1.74 |
| SpMCh31 | 18.09 | 3.37 | 12.80 | 2.70 |
| SpMCh32 | 49.14 | 4.62 | 20.69 | 1.75 |
| SpMCh33 | 47.28 | 9.66 | 40.09 | 9.20 |
| SpMCh34 | 15.51 | 3.28 | 10.54 | 2.22 |
| SpMCh35 | 16.53 | 0.51 | 2.83 | 0.39 |
| SpMCh36 | 60.40 | 7.70 | 42.63 | 6.74 |
| SpMCh37 | 33.75 | 7.43 | 22.15 | 4.62 |
| SpMCh38 | 3.83 | 1.44 | 1.93 | 0.94 |
| SpMCh39 | 8.38 | 0.19 | 7.43 | 0.15 |
| SpMCh40 | 36.23 | 4.45 | 33.25 | 4.32 |
| SpMCh41 | 50.93 | 4.93 | 29.85 | 4.31 |
| SpMCh42 | 48.11 | 4.11 | 45.10 | 4.44 |
| SpMCh43 | 45.09 | 15.63 | 20.23 | 3.98 |
| SpMCh44 | 59.19 | 17.82 | 57.08 | 19.28 |
| SpMCh45 | 58.89 | 5.93 | 48.23 | 5.43 |
| SpMCh46 | 69.33 | 5.99 | 60.30 | 6.03 |
| SpMCh47 | 91.60 | 0.27 | 77.80 | 0.67 |
| SpMCh48 | 20.01 | 2.93 | 17.55 | 2.72 |
| SpMCh49 | 43.63 | 5.81 | 20.69 | 3.57 |
| SpMCh50 | 65.51 | 5.25 | 52.14 | 4.27 |
| SpMCh51 | 49.14 | 3.23 | 38.46 | 3.74 |
| SpMCh52 | 75.29 | 12.26 | 72.51 | 13.77 |
| SpMCh53 | 57.81 | 3.59 | 55.90 | 4.63 |
| SpMCh54 | 48.61 | 7.92 | 38.83 | 7.18 |
| SpMCh55 | 80.03 | 6.19 | 35.04 | 2.26 |
| SpMCh56 | 32.06 | 4.51 | 28.75 | 3.75 |
| SpMCh57 | 2.56 | 1.15 | 1.89 | 0.82 |
| SpMCh58 | 1.97 | 0.33 | 1.47 | 0.51 |
| SpMCh59 | 56.31 | 1.26 | 47.10 | 1.30 |
| SpMCh60 | 55.51 | 3.10 | 40.49 | 2.68 |
| SpMCh61 | 79.65 | 2.83 | 73.28 | 1.46 |

*Frameshift INDEL % refers to INDELs expected to result in a frameshift mutation in the FAAH coding sequence (i.e., ±1 nt, ±2 nt, ±4 nt). Thus, Frameshift INDEL % refers to frequency of total INDELs minus frequency of INDELs that are ±3 nt, ±6 nt, ±9 nt, etc. All data entered are a mean of n = 3 replicate

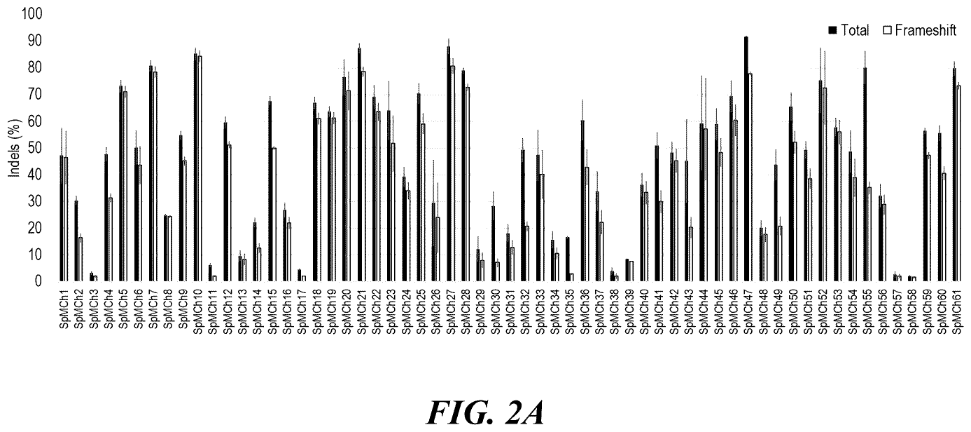

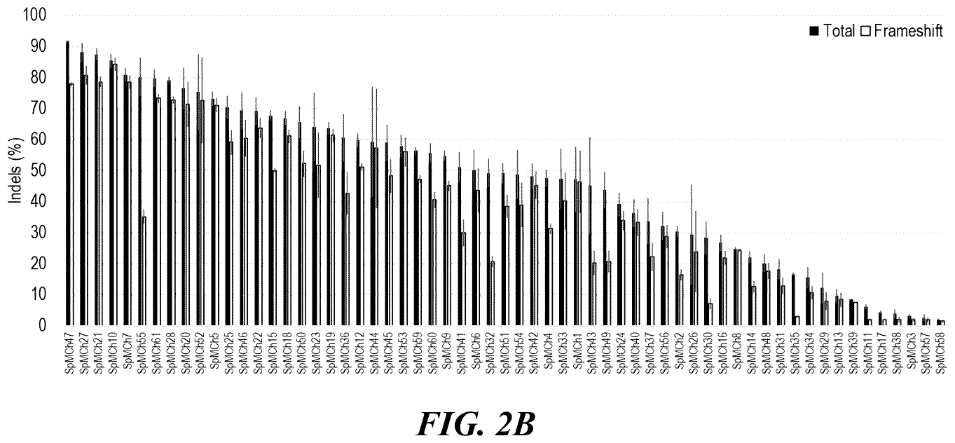

The results of quantification of editing efficiency of SpCas9 sgRNAs targeting MYOC coding sequence are shown in FIGS. 2A-2B. FIG. 2A illustrates the quantification ranked by guide numbering, and FIG. 2B illustrates the quantification ranked by INDEL percentages. As shown therein, ribonucleoprotein complexes of SpCas9 protein and one of 61 SpCas9 sgRNAs were transfected in HEK293T cells and cells harvested at a later time point. Following DNA extraction, PCR and TIDE were performed to determined percentage of indels at cut site. Total editing percentage ranged from 91.60% for SpMCh47 to 1.98% for SpMCh58 in conditions tested. The highest frameshift percentage was observed SpMCh 10 sgRNA transfected sample with 84.23% (85.24% total indels).

### Example 2: In Vitro Lipid Nanoparticles Screening

This example describes in vitro efficacy studies of lipid nanoparticle (LNP) delivery via different LNPs.

Several human primary cells and one cell line were used for the studies, which included primary trabecular meshwork (TM) cells and an immortalized glaucomatous TM cell line (GTM3) expressing myocilin mutant (G364V) protein in fusion with dsRED. All LNPs were formulated with GFP mRNA and delivered to the cells. Pictures were taken at same timepoint for all formulations tested and GFP score was determined using no GFP signal as score of 0 and highest GFP signal as score of 3.

The characteristics and formulations of the LNPs used in these studies are shown in Table 8 (LNPs obtained from Source 1) and Table 9 (LNPs obtained from Source 2).

TABLE 8
Characteristics and Formulations of Lipid Nanoparticles Tested

| | Size | | |
|---|---|---|---|
| Name | (nm) | PDI | Formulation |
| CTX-DLIN-1 | 196.2 | 0.29 | 0.5/39.5/10/50, mol % DMG- |
| | | | PEG/Chol/DSPC/DLIN-MC3 |
| CTX-DLIN-2 | 200.5 | 0.06 | 1.5/38.5/10/50, mol % DMG- |
| | | | PEG/Chol/DSPC/DLIN-MC4 |
| CTX-DLIN-3 | 96.1 | 0.07 | 3/37.5/10/50, mol % DMG- |
| | | | PEG/Chol/DSPC/DLIN-MC5 |
| CTX-C12-1 | 207.3 | 0.1 | 0.5/39.5/10/50, mol % DMG- |
| | | | PEG/Chol/DSPC/C12-200 |
| CTX-C12-2 | 146.4 | 0.09 | 1.5/38.5/10/50, mol % DMG- |
| | | | PEG/Chol/DSPC/C12-200 |
| CTX-C12-3 | 134.4 | 0.06 | 3/37.5/10/50, mol % DMG- |
| | | | PEG/Chol/DSPC/C12-200 |
| CTX-DOD-1 | 1462.3 | 0.59 | 0.5/39.5/10/50, mol % DMG- |
| | | | PEG/Chol/DSPC/DODMA |
| CTX-DOD-2 | 190.9 | 0.04 | 1.5/38.5/10/50, mol % DMG- |
| | | | PEG/Chol/DSPC/DODMA |
| CTX-DOD-3 | 75.9 | 0.15 | 3/37.5/10/50, mol % DMG- |
| | | | PEG/Chol/DSPC/DODMA |
| CTX-DOT-1 | 75.5 | 0.13 | 0.5/39.5/10/50, mol % DMG- |
| | | | PEG/Chol/DSPC/DOTAP |
| CTX-DOT-2 | 72.3 | 0.17 | 1.5/38.5/10/50, mol % DMG- |
| | | | PEG/Chol/DSPC/DOTAP |
| CTX-DOT-3 | 60.5 | 0.21 | 3/37.5/10/50, mol % DMG- |
| | | | PEG/Chol/DSPC/DOTAP |
| CTX-C12-CT | 195.3 | 0.06 | 1.0/45.0/16.0/38.0, |
| | | | mol % DMG-PEG/ |
| | | | Sitosterol/DSPC/C12-200 |

* As described herein, “CTX-C12-200-CT” is a non-limiting example of CTX-C12-CT. Size of the LNP in the LNP formulations described herein (e.g., CTX-C12-CT LNP formulation) can vary. For example, the particle size of the LNP in the CTX-C12-CT formulation can be in 85-95 nm.

TABLE 9
Characteristics of Lipid Nanoparticles from Different Sources

| | Size | | |
|---|---|---|---|
| Name | (nm) | PDI | Formulation |
| LNP-P1 | 113 | 0.11 | Undisclosed |
| LNP-P2 | 105 | 0.14 | Undisclosed |
| LNP-P3 | 113 | 0.15 | Undisclosed |
| LNP-P4 | 97 | 0.07 | Undisclosed |
| LNP-P5 | 88 | 0.04 | Undisclosed |
| LNP-P6 | 83 | 0.05 | Undisclosed |
| LNP-P7 | 87 | 0.07 | Undisclosed |
| LNP-P8 | 99 | 0.1 | Undisclosed |
| LNP-P9 | 116 | 0.15 | Undisclosed |
| LNP-P10 | 99 | 0.12 | Undisclosed |
| LNP-P11 | 118 | 0.09 | Undisclosed |
| LNP-P12 | 111 | 0.1 | Undisclosed |
| LNP-P13 | 141 | 0.05 | Undisclosed |
| LNP-A14 | 66 | 0.09 | Undisclosed |

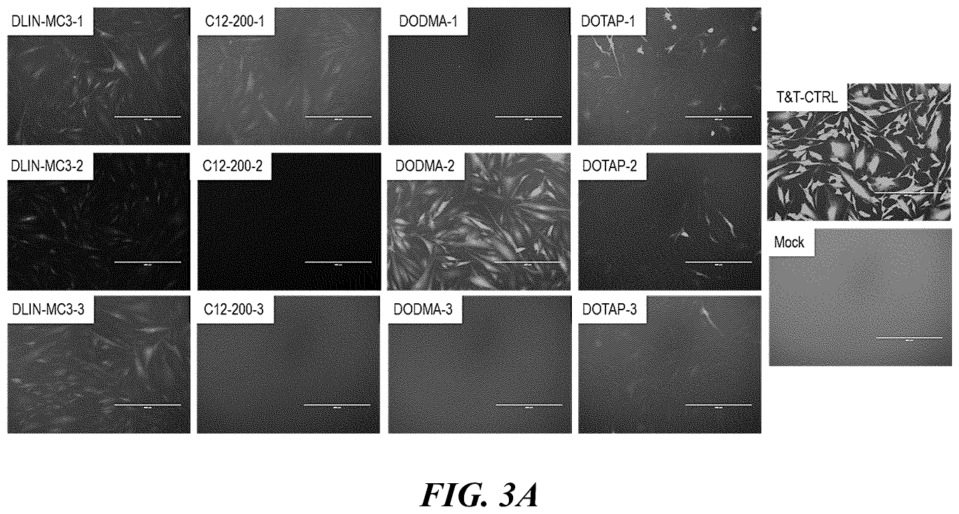

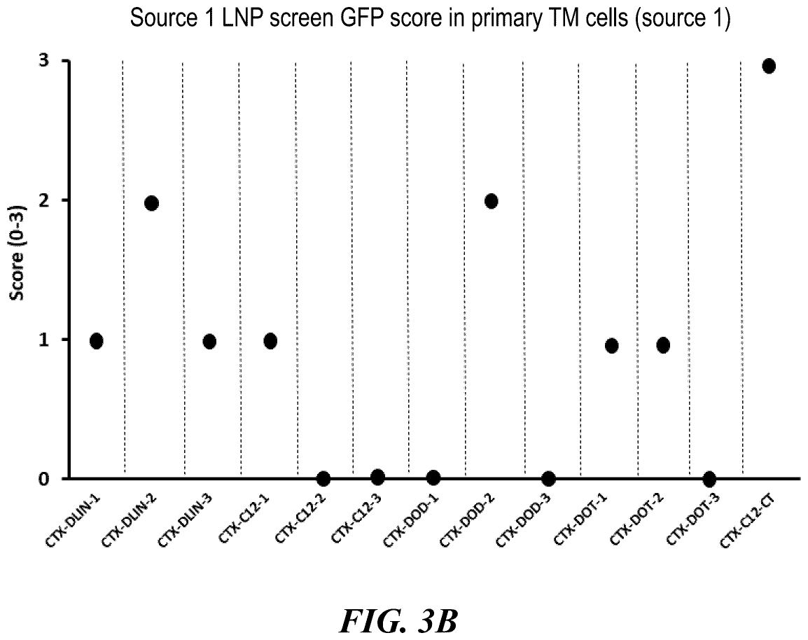

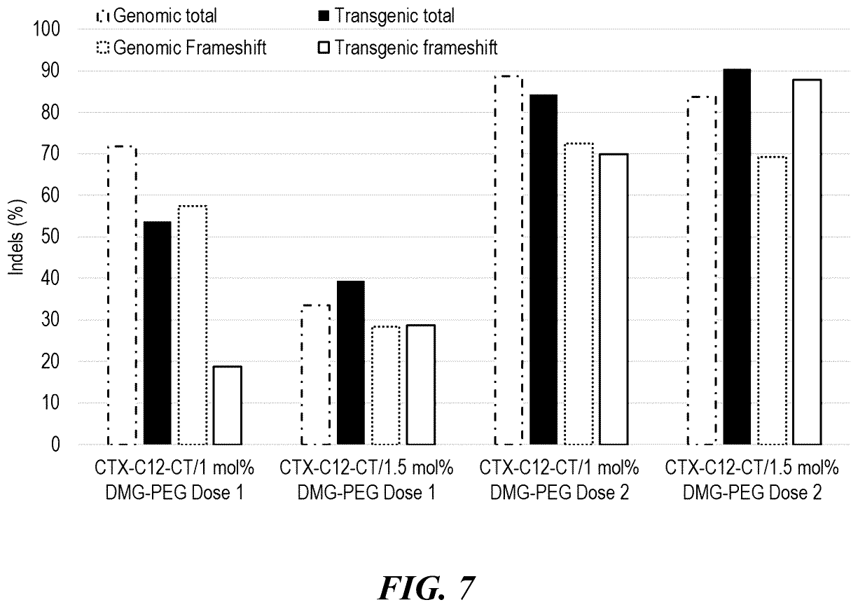

The results are shown in FIGS. 2A/B-6A/B and FIG. 7. Specifically, FIGS. 3A-3B illustrate screening of LNPs obtained from Source 1 in primary TM cells. LNPs carrying GFP mRNA were transfected in primary TM cells in culture. At 22 hours post-transfection, pictures were taken (FIG. 3A) and GFP score was determined (FIG. 31B).

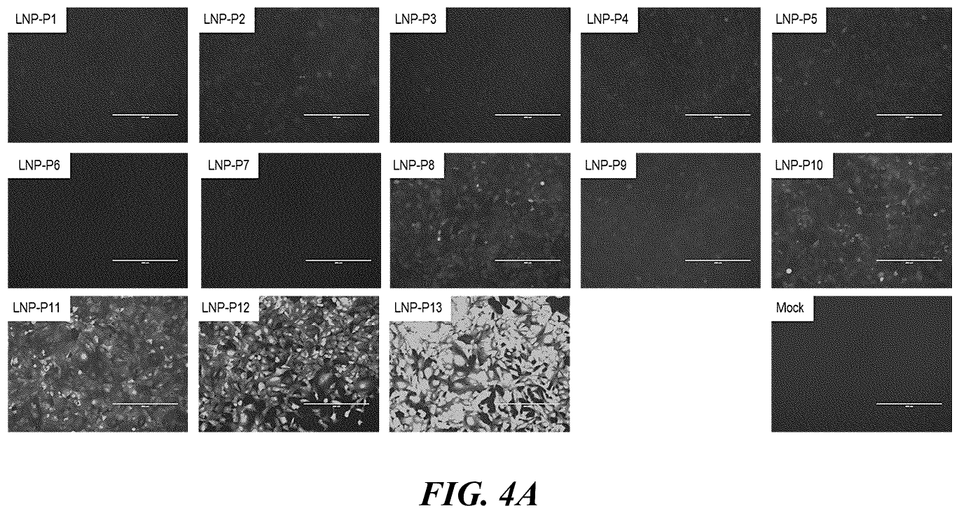

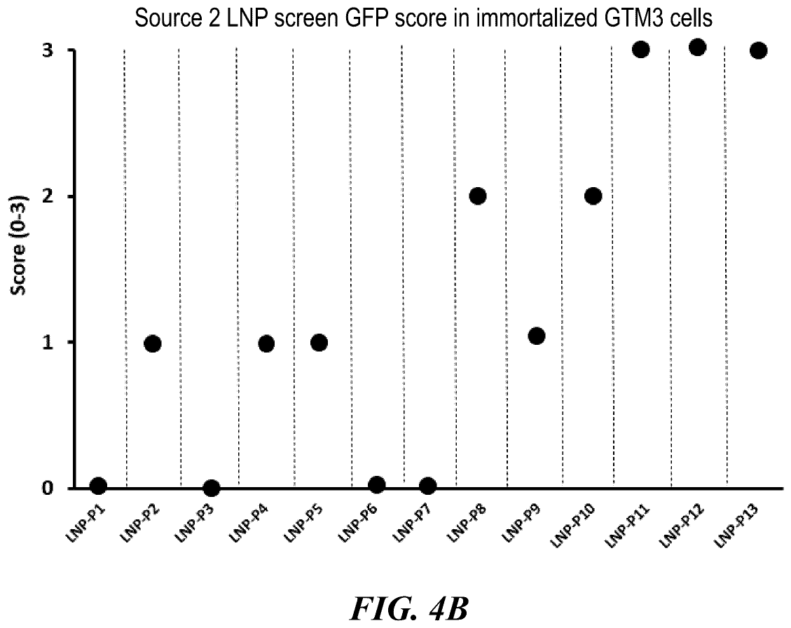

FIGS. 4A-4B illustrate screening of LNPs obtained from Source 2 in GTM3 cell line. LNPs carrying GFP mRNA were transfected in immortalized glaucomatous TM cells in culture. GTM3 cells carried a transgenic form of mutant G364V′ myocilin in fusion with dsRED (not shown in pictures). At 22 hours post-transfection, pictures were taken (FIG. 4A) and GFP score was determined (FIG. 4B).

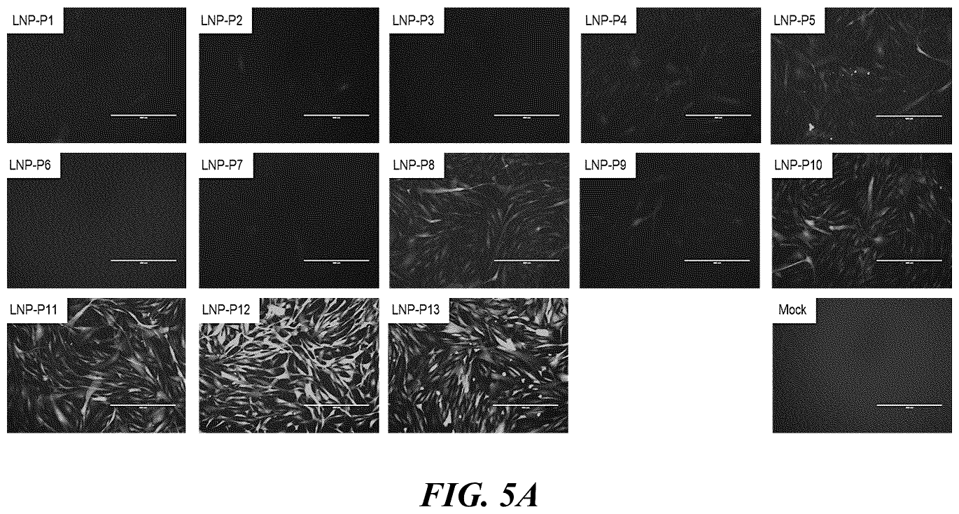

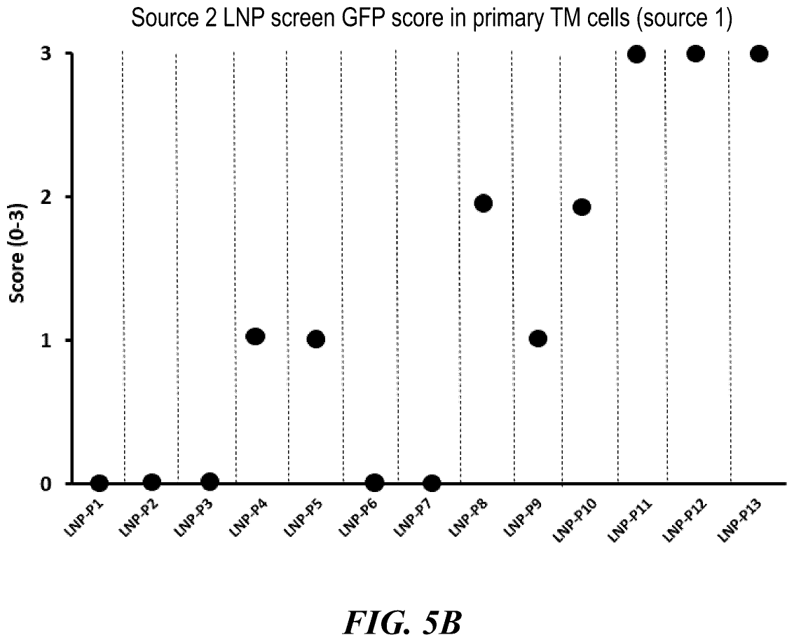

FIGS. SA-5B illustrate screening of LNPs obtained from Source 2 in primary TM cells obtained from Source 1. LNPs carrying GFP mRNA were transfected in primary TM cells in culture. At 22 hours post-transfection, pictures were taken (FIG. 5A) and GFP score was determined (FIG. 5B).

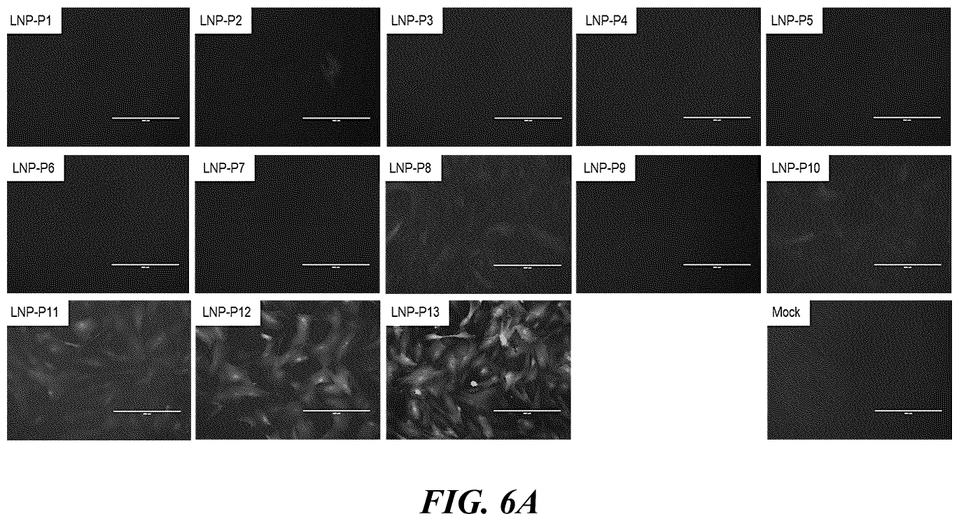

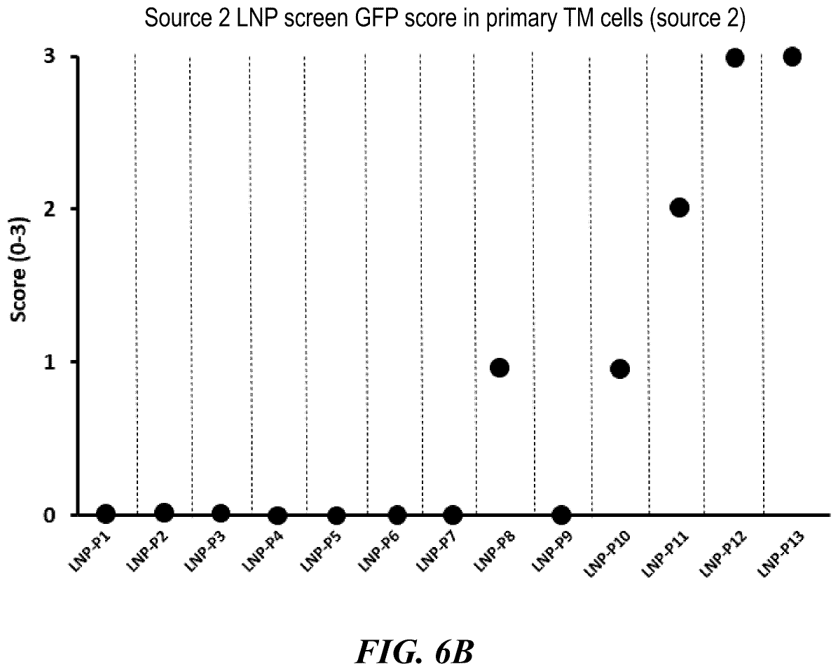

FIGS. 6A-6B illustrate screening of LNPs obtained from Source 2 in primary TM cells obtained from Source 2. LNPs carrying GFP mRNA were transfected in primary TM cells in culture. At 22 hours post-transfection, pictures were taken (FIG. 6A) and GFP score was determined (FIG. 6B).

Several different sources of trabecular meshwork (TM) cells were used for LNP screening as TM cells are notoriously difficult to isolate and can be contaminated from other cell types. By using multiple sources, a pattern could be confirmed for LNP transfection efficacy in TM cells.

FIG. 7 illustrates the results of quantification of editing efficiency of selected SpCas9 sgRNAs targeting MYOC coding sequence using selected lipid nanoparticles (LNPs) for delivery in GTM3 cells. GTM3 cells were transfected with either one of the two (2) versions of the CTX-C12-200-CT from Source 1: (1) containing 1 mol % DMG-PEG; or (2) containing 1.5 mol % DMG-PEG. Both versions were formulated with Cas9 protein and SpMCh21 sgRNAs. Two different doses were transfected in GTM3 cells which were harvested 2 days post transfection, DNA extracted, and PCR and TIDE performed to determined total indels and frameshift indels percentages at both genomic and transgenic sites. At the lower doses, 1 mol % DMG-PEG performed slightly better than 1.5 mol % DMG-PEG. However, at higher dose, they performed similarly, suggesting a plateau effect at such dose. The experiment was to confirm the efficacy of one of the leading performing LNP formulations tested with Cas9/sgRNA to achieve editing in cell model in vitro.

### Example 3: In Vivo Lipid Nanoparticles Screening

This example describes screening of LNPs in vivo. BALB/c mice were used for in vivo studies. One (1) microliter containing LNPs formulated with GFP mRNA was injected by intravitreal injection and whole globes were isolated at a later time point (e.g., at 24-hour timepoint). Whole globes were fixed, embedded, and processed for immunohistochemistry staining for GFP expression. Pictures were taken at same timepoint for all formulations tested and GFP score was determined using no GFP signal as score of 0 and highest GFP signal as score of 3.

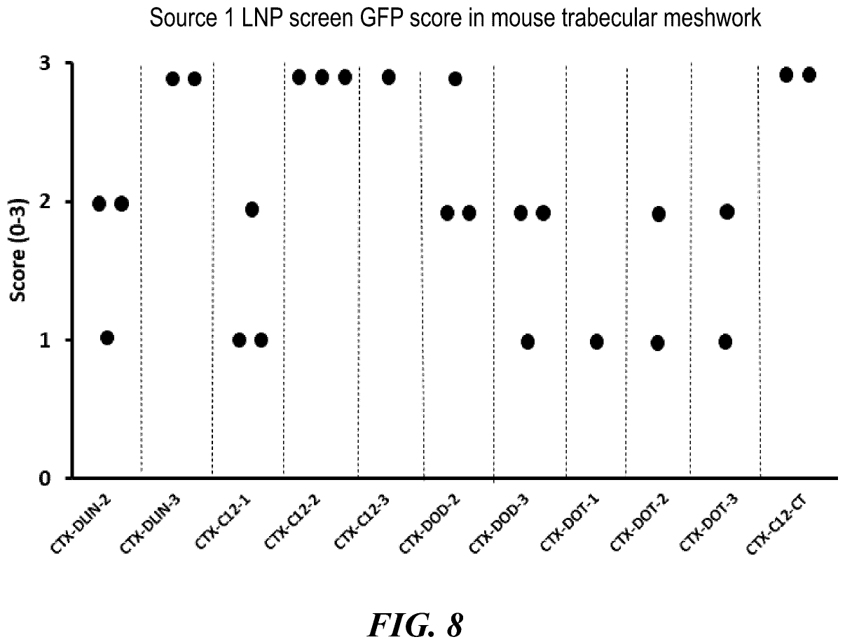

FIG. 8 illustrates GFP score of LNPs obtained from Source 1 in mouse trabecular meshwork following intravitreal injection of 300 ng of LNPs. LNPs carrying GFP mRNA were injected intravitreally in the eye of BALB/c mice. At 5 hours, mice were euthanized, and their eyes processed for IHC and anti-GFP staining. Pictures were taken and GFP score was determined.

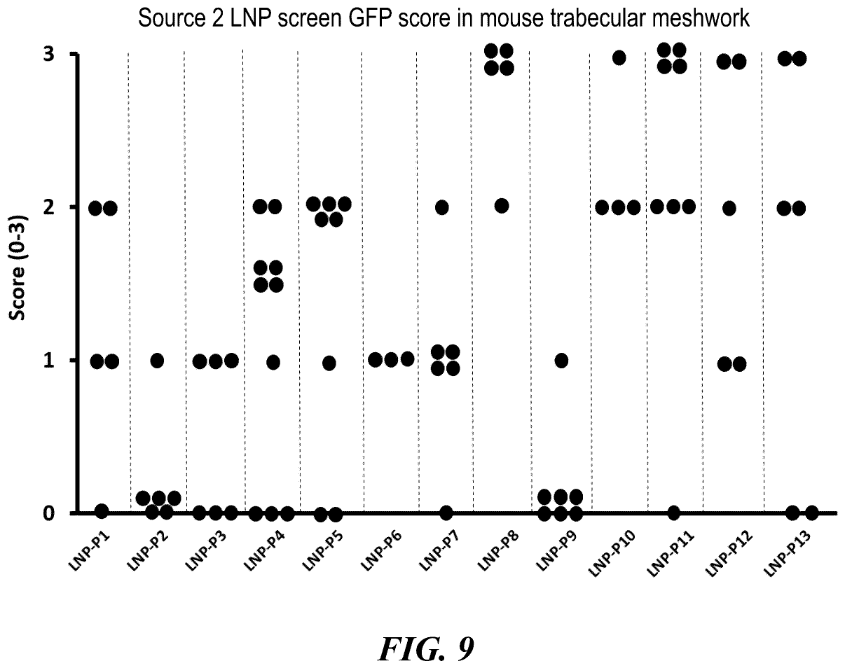

FIG. 9 illustrates GFP score of LNPs obtained from Source 2 in mouse trabecular meshwork following intravitreal injection of 300 ng of LNPs. LNPs carrying GFP mRNA were injected intravitreally in the eye of BALB/c mice. At 5 hours, mice were euthanized, and their eyes processed for IHC and anti-GFP staining. Pictures were taken and GFP score was determined.

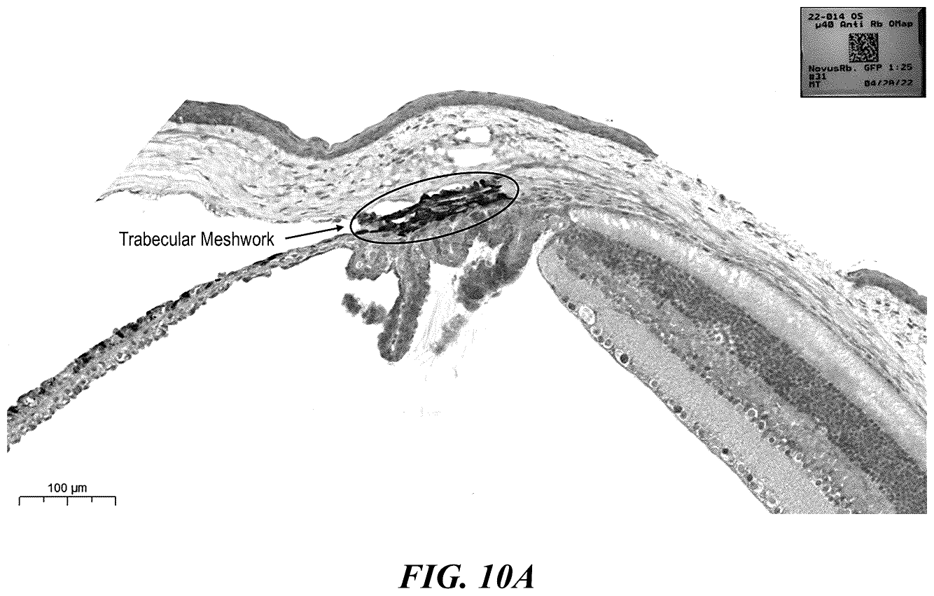

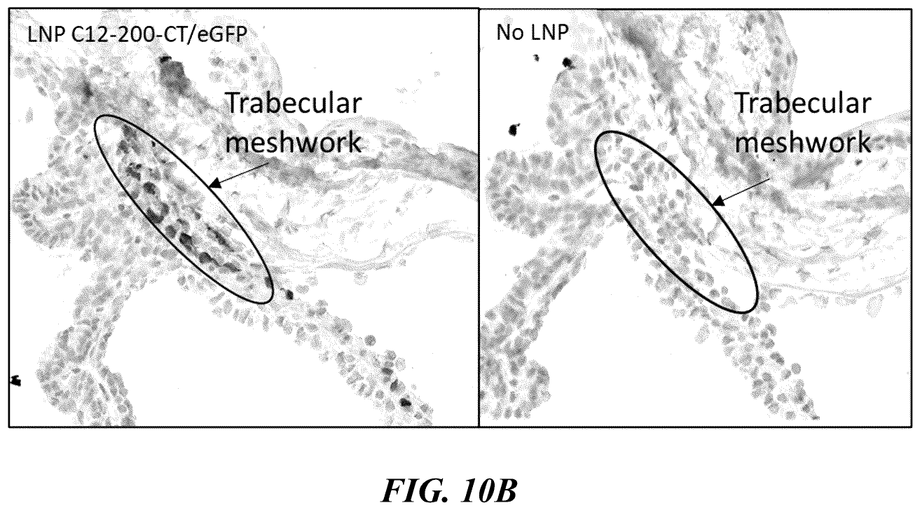

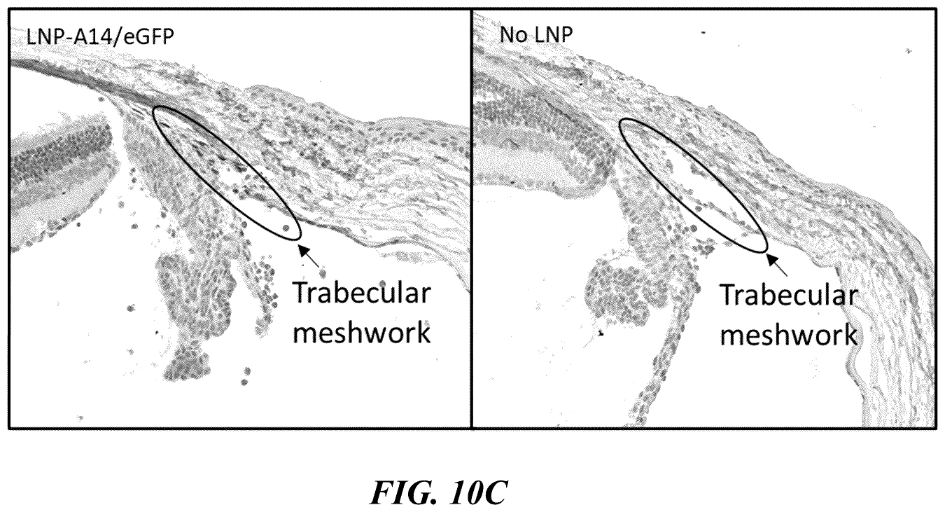

FIG. 10A illustrates IHC GFP staining of delivery of CTX-C12-CT (described in Table 8) obtained from Source 1 in mouse trabecular meshwork following intravitreal injection of 300 ng of LNP. GFP expression was seen in the trabecular meshwork tissue of this cross-section of a Balb/c mouse eye 5 hours post-injection of CTX-C12-CT formulated with GFP mRNA. FIG. 10B illustrates GFP protein expresses in the mouse trabecular tissue after intravitreal injection of LNP CTX-C12-200-CT/eGFP mRNA in a mouse eye. FIG. 10C illustrates GFP protein expresses in the mouse trabecular tissue after intravitreal injection of LNP A14/eGFP mRNA in a mouse eye. These results are the proof-of-concept that the intravitreal injection of LNP in the eye results in the expression of the cargo in the trabecular meshwork tissue.

### Example 4: In Vitro LNP Delivery of Cas9 and sgRNA

This example describes LNP delivery of Cas9 mRNA and sgRNA in vitro. The immortalized human glaucomatous TM cell line expressing myocilin mutant (G364V or Y437H) protein in fusion with dsRED was transfected with LNPs formulated with Cas9 mRNA and sgRNA for MYOC targeting. CTX-C12-CT with either 1% or 1.5% DMG-PEG LNP formulations were used to deliver Cas9 mRNA and sgRNA.

The primary human trabecular meshwork cells were transfected with LNPs formulated with Cas9 mRNA and sgRNA for MYOC targeting for 2 days, then cells were treated with dexamethasone to induce myocilin expression and harvested 3 days later.

The genomic DNA was extracted and subjected to TIDE (tracking of indels by decomposition) analyses. The results show that the combination of one of the lead sgRNA (SpMCh21) and one of the best LNP (CTX-C12-CT) can achieve high level of editing in trabecular meshwork cells.

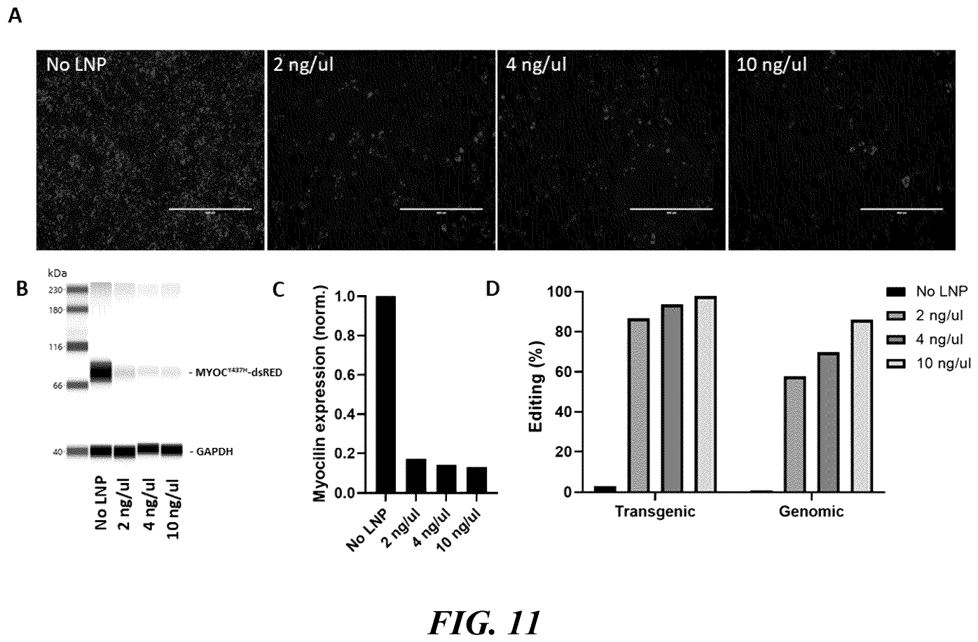

MYOC gene editing and myocilin protein knockdown after delivery of LNP CTX-C12-200-CT/Cas9 mRNA/SpMCh10 sgRNA to the human glaucomatous trabecular meshwork cell line GTM3 MYOC^{Y437H}-dsRED are demonstrated in FIG. 11. In this study, the human glaucomatous GTM3 MYOC^{Y437H}-dsRED were transfected with different amounts of LNP CTX-C12-200-CT formulated with S. pyogenes Cas9 mRNA and SpMCh10 targeting the human MYOC gene. The GTM3 MYOC^{Y437H}-dsRED cell line is an immortalized human trabecular meshwork cell line stably expressing the transgene myocilin mutant Y437H in fusion with the reporter protein dsRED.

It is shown that targeting the myocilin mutant sequence impacted the expression of the fusion protein, which is shown by the reduction of the dsRED expression in edited samples observed by epifluorescence microscopy (FIG. 11, panel A). Proteins were extracted at day 5 post-transfection and immunoblot was performed using anti-GAPDH and anti-myocilin detecting myocilin in fusion with dsRED (FIG. 11, panels B and C). DNA was also extracted and PCR and TIDE were performed to determined percentage of indels at cut site for both genomic and transgenic locus (FIG. 11, panel D). Myocilin mutant-dsRED protein expression is linked to the transgenic expression of MYOC and while genomic MYOC was also edited, no wild-type expression of myocilin was detected in this cell line. Editing percentages at the transgenic locus were ranging from 86.7% (2 ng/μl) to 97.8% (10 ng/μl) (FIG. 11, panel D), resulting in myocilin expression reduction by 83% (2 ng/μl) to 87% (10 ng/μl) (FIG. 11, panels B-C), compared to the non-edited control. These results suggest that high editing percentages of the MYOC gene leads to high myocilin protein knock down.

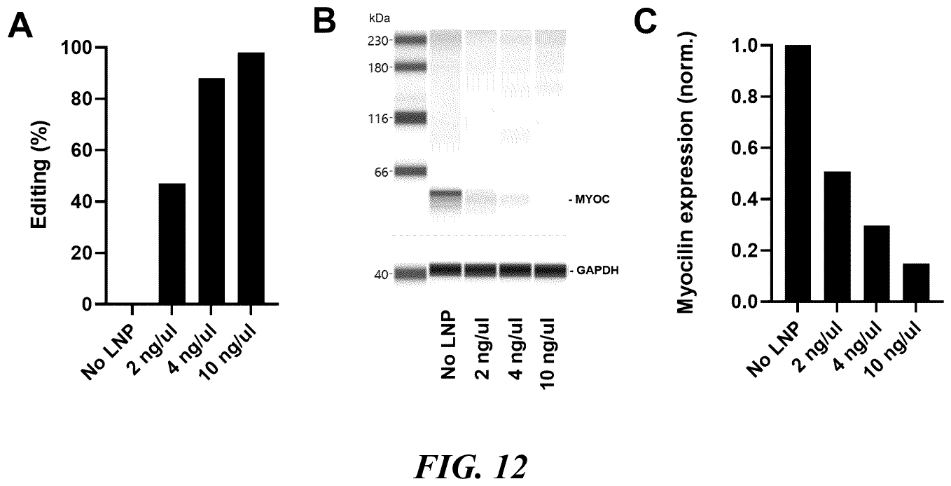

MYOC gene editing and myocilin protein knockdown after delivery of LNP CTX-C12-200-CT/Cas9 mRNA/SpMCh10 sgRNA to human primary trabecular meshwork cells are demonstrated in FIG. 12. In this study, human primary trabecular meshwork cells were transfected with different amounts of LNP CTX-C12-200-CT formulated with S. pyogenes Cas9 mRNA and SpMCh10 targeting the MYOC gene. Cells were transfected for 2 days then dexamethasone was added to the media to induce expression of myocilin. Three (3) days later, samples were harvested. DNA was extracted and PCR and TIDE were performed to determined percentage of indels at cut site for the genomic locus (FIG. 12, panel A). Proteins were extracted and immunoblot was performed using anti-GAPDH and anti-myocilin detecting wild type myocilin (FIG. 12, panels B and C). Editing percentages at the genomic locus ranging from 47% (2 ng/μl) to 98% (10 ng/μl) (FIG. 12, panel A) result in myocilin expression reduction by 49% (2 ng/μl) to 85% (10 ng/μl) (FIG. 12, panels B and C), compared to the non-edited control. These results suggest the correlation between editing percentages at MYOC gene locus and myocilin protein knock down.

### Example 5: Ex Vivo LNP Delivery of Cas9 and sgRNA

This example describes LNP delivery of Cas9 mRNA and MYOC sgRNA ex vivo. Anterior segment organ cultures (ASOC) were established from adult deceased human donors with no glaucomatous or reported eye conditions. ASOC were perfused with serum containing media at a flow rate of 2.5 μl/ml and intraocular pressure monitored. 50 μg of LNP CTX-C12-200-CT/eGFP or LNP CTX-C12-200-CT/Cas9 mRNA/MYOC sgRNAs were transfected for 24h (GFP) or 4 days (Cas9/sgRNAs) by introducing the formulations using a syringe and pump system. For GFP, ASOC were fixed, embedded, and processed for immunohistochemistry (IHC) staining for GFP expression. For Cas9 mRNA/sgRNAs infusions, tissues (cornea, sclera, and trabecular meshwork) were isolated and DNA or protein isolated for TIDE or western blot analysis).

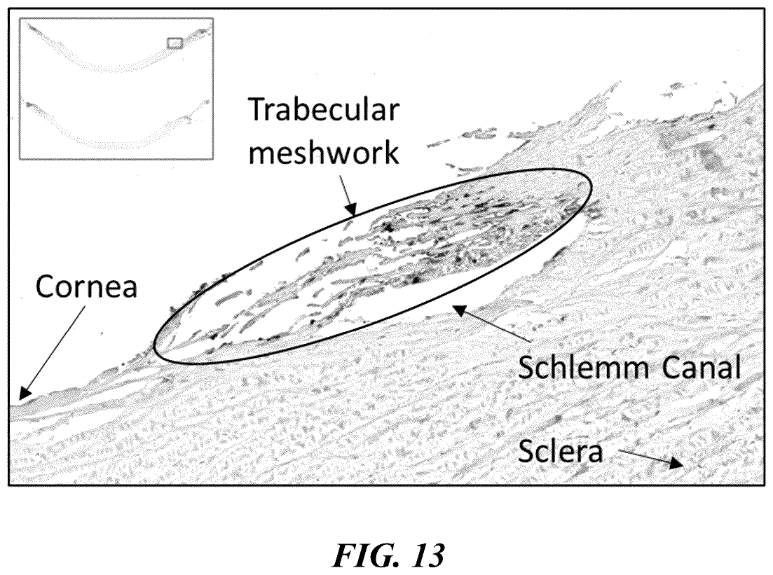

GFP protein expression in the trabecular meshwork tissue after delivery of LNP CTX-C12-200-CT/eGFP mRNA in an ex vivo anterior segment organ culture (ASOC) is demonstrated in by IHC staining in FIG. 13. The result suggests high and specific expression of GFP in the trabecular meshwork tissue after LNP transfection.

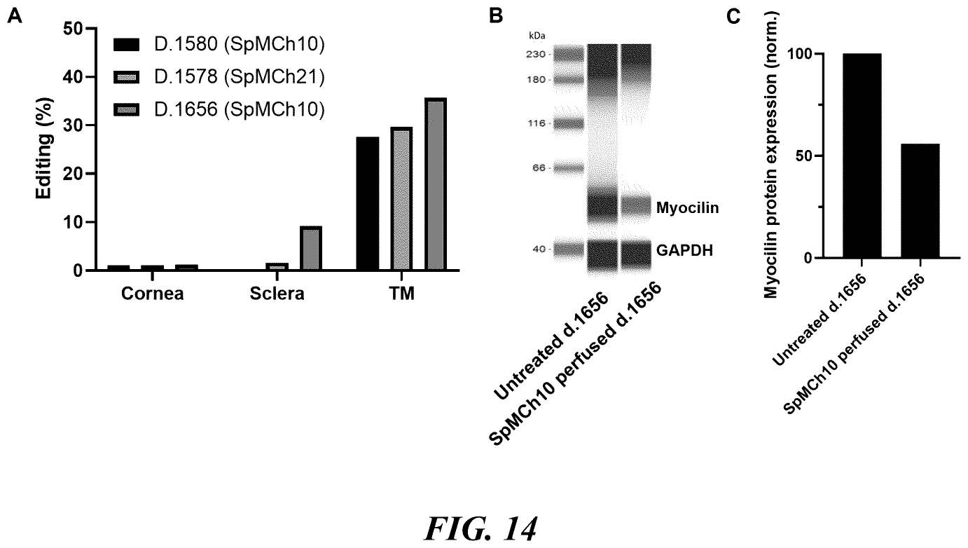

MYOC gene editing and myocilin protein knockdown in the trabecular meshwork tissue after delivery of LNP CTX-C12-200-CT/Cas9 mRNA/MYOC sgRNAs in an ex vivo anterior segment organ culture (ASOC) are demonstrated in FIG. 14. An ASOC was established from whole globes received from human decease donors. 50 μg of LNP CTX-C12-200-CT formulated with S. Pyogenes Cas9 mRNA and MYOC sgRNAs (either SpMCh10 or SpMCh21) were injected in the perfusion system at a flow rate of 2.5 μl/ml. Four (4) days later, cornea, sclera and trabecular meshwork tissues were isolated from the ASOC. DNA was extracted and PCR and TIDE were performed to determined percentage of indels at cut site for at the genomic locus (FIG. 14, panel A). Proteins from experiment involving donor #1656 were extracted and immunoblot was performed using anti-GAPDH and anti-myocilin detecting wild type myocilin (FIG. 14, panels B and C). Editing percentages in trabecular meshwork were respectively 27.6%, 29.7% and 35.7% for donors 1580, 1578 and 1656; where editing percentages in sclera and cornea were low (1-9%) (FIG. 14, panel A). Myocilin protein expression monitored in donor 1656 shows a 44% protein reduction (FIG. 12, panel B and FIG. 11, panel C) compared to the contralateral non-edited tissue control. These results are proof-of-concept that MYOC gene editing in the target trabecular meshwork tissue results in downregulation of the myocilin protein in that tissue.

### Example 6: In Vivo LNP Delivery of Cas9 and sgRNA

This example describes LNP delivery of Cas9 mRNA and sgRNA in vivo in mouse eye. ACTA2/α-SMA was chosen as a non-limiting exemplary target gene because this protein is well express in the trabecular meshwork and can be easily monitored by IHC.

One (1) microliter containing CTX-C12-CT LNP formulated with Cas9/sgRNA was injected intravitreally into BALB/c mouse eyes. The sgRNA targeting the mouse ACTA2 gene (α-smooth muscle actin or SMA) was selected for high editing efficiency in vitro. Whole globes of Naïves and LNP injected mice were harvested one (1), two (2) and four (4) weeks post-injection. Whole globes were fixed, embedded and processed for immunohistochemistry staining fora-SMA expression. Presence of SMA within the trabecular mesh work was evaluated at six (6) locations within the eye. A score from 0 (no TM cells with SMA signal) to 3 (majority of TM cells with SMA signal) was assigned to each of the six (6) locations and an average score was determined for each eye.

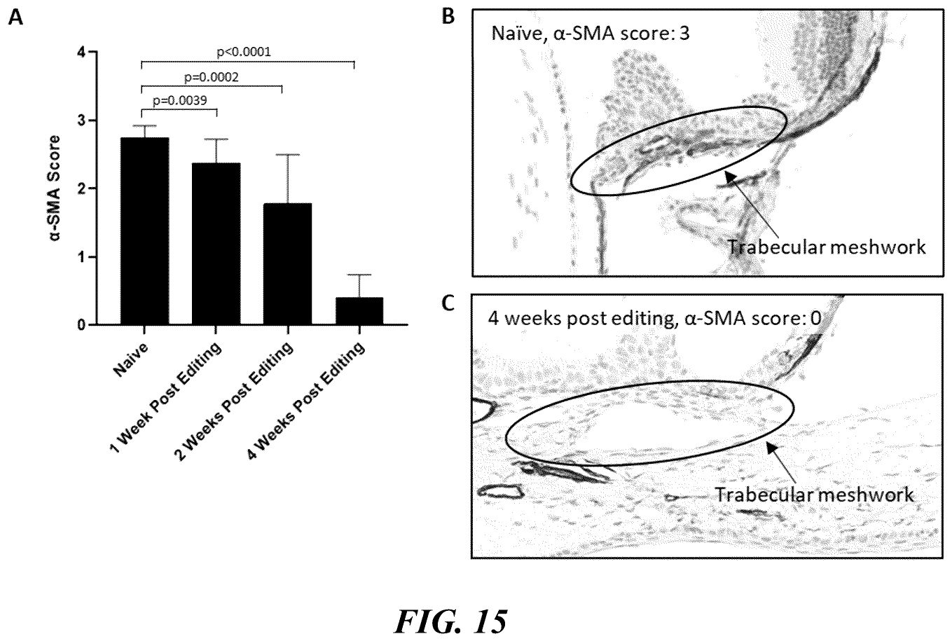

Following one injection of LNP containing sgRNAs targeting the ACTA2 mouse gene, the expression of the ACTA2 gene protein, α-SMA, decreased continuously from week 1 to week 4 as shown in FIG. 15, panel A). IHC were scored for the expression and the number of cells expressing α-SMA. FIG. 15, panel A shows an example of a score of 3 (highest score) in naïves/untreated animal, and FIG. 15, panel B shows an example of a score of 0 (lowest score) in the trabecular meshwork of a treated animals 4 four weeks post LNP injection.

This example demonstrates that LNP can deliver Cas9 mRNA/sgRNA in vivo to the trabecular meshwork and results in efficient editing by the Cas9/sgRNA complex at the target sequence and downregulation of the protein expression.

### Sequence Tables

The following tables provide details for the various nucleotide and amino acid sequences disclosed herein.

TABLE 1
Target Sequences for SaCas9 sgRNAs in the MYOC Coding Sequence

| | Target Sequence | SEQ ID NO | SEQ ID NO | Cut site |
|---|---|---|---|---|
| Name | (PAM in bold underline) | (no PAM) | (with PAM) | Location* |
| SaMCh1 | CTGCTTCTGGCCTGCCTGGTGTGGGAT | 1 | 28 | 171652533 |
| SaMCh2 | CAGCTCAGGAAGGCCAATGACCAGAGT | 2 | 29 | 171652488 |
| SaMCh3 | CCTTCAGTGTGGCCAGTCCCAATGAAT | 3 | 30 | 171652445 |
| SaMCh4 | GCCTGGCTCTGCTCTGGGCAGCTGGAT | 4 | 31 | 171652429 |
| SaMCh5 | GCTGCTGTCTCTCTGTAAGTTATGGAT | 5 | 32 | 171652392 |
| SaMCh6 | GGTGGCCTCCAGGTCTAAGCGTTGGGT | 6 | 33 | 171652365 |
| SaMCh7 | TGGAGGAGGCTCTCCAGGGAGCTGAGT | 7 | 34 | 171652330 |
| SaMCh8 | CAGCTCCCTCTGCAGCCCCTCCTGGGT | 8 | 35 | 171652266 |
| SaMCh9 | CAGCTGGTCCCGCTCCCGCCTCAGGGT | 9 | 36 | 171652236 |
| SaMCh10 | ACCAGCTGGAAACCCAAACCAGAGAGT | 10 | 37 | 171652208 |
| SaMCh11 | GGCAGTCTCCAACTCTCTGGTTTGGGT | 11 | 38 | 171652206 |
| SaMCh12 | AAGCGACTAAGGCAAGAAAATGAGAAT | 12 | 39 | 171652125 |
| SaMCh13 | TGGCACAGCCCGAGCAGTGTCTCGGGT | 13 | 40 | 171652041 |
| SaMCh14 | GCTAACTGAAGTTCCTGCTTCCCGAAT | 14 | 41 | 171638655 |
| SaMCh15 | GAGAGCCCATCTGGCTATCTCAGGAGT | 15 | 42 | 171638621 |
| SaMCh16 | AATTACTGGCAAGTATGGTGTGTGGAT | 16 | 43 | 171636636 |
| SaMCh17 | GGGTGTAGGGGTAGGTGGGCTTGGGGT | 17 | 44 | 171636613 |
| SaMCh18 | CTACACCCAGGAGACCACGTGGAGAAT | 18 | 45 | 171636585 |
| SaMCh19 | TCCACGTGGTCTCCTGGGTGTAGGGGT | 19 | 46 | 171636598 |
| SaMCh20 | GTCGATTCTCCACGTGGTCTCCTGGGT | 20 | 47 | 171636590 |
| SaMCh21 | TGGAGAATCGACACAGTTGGCACGGAT | 21 | 48 | 171636566 |
| SaMCh22 | GCACGGATGTCCGCCAGGTTTTTGAGT | 22 | 49 | 171636547 |
| SaMCh23 | TAGGCAGTATGTGAACCTTAGAAGGGT | 23 | 50 | 171636499 |
| SaMCh24 | CTGCCTAGGCCACTGGAAAGCACGGGT | 24 | 51 | 171636470 |
| SaMCh25 | GGAGCCTCTATTTCCAGGGCGCTGAGT | 25 | 52 | 171636427 |
| SaMCh26 | CGCCCTGGAAATAGAGGCTCCCCGAGT | 26 | 53 | 171636442 |
| SaMCh27 | AGAACTGTCATAAGATATGAGCTGAAT | 27 | 54 | 171636398 |

*Chromosomal location of guide cut-site in chromosome 1 of human genome Hg

TABLE 2
Target Sequences for SpCas9 sgRNAs in the MYOC Coding Sequence

| | Target Sequence | SEQ ID NO | SEQ ID NO | Cut site |
|---|---|---|---|---|
| Name | (PAM in bold underline) | (no PAM) | (with PAM) | Location* |
| SpMCh1 | GTGCACGTTGCTGCAGCTTTGGG | 55 | 116 | 171652581 |
| SpMCh2 | AGCTGGACAGCTGGCATCTCAGG | 56 | 117 | 171652569 |
| SpMCh3 | AGCTGTCCAGCTGCTGCTTCTGG | 57 | 118 | 171652547 |
| SpMCh4 | GCTTCTGGCCTGCCTGGTGTGGG | 58 | 119 | 171652532 |
| SpMCh5 | GCCTGCCTGGTGTGGGATGTGGG | 59 | 120 | 171652525 |
| SpMCh6 | CTGCCTGGTGTGGGATGTGGGGG | 60 | 121 | 171652523 |
| SpMCh7 | GGGCCAGGACAGCTCAGCTCAGG | 61 | 122 | 171652503 |
| SpMCh8 | GCATCGGCCACTCTGGTCATTGG | 62 | 123 | 171652487 |
| SpMCh9 | TATACTGGCATCGGCCACTCTGG | 63 | 124 | 171652480 |
| SpMCh10 | ATGCCAGTATACCTTCAGTGTGG | 64 | 125 | 171652457 |
| SpMCh11 | CACTGAAGGTATACTGGCATCGG | 65 | 126 | 171652471 |
| SpMCh12 | TGGCCACACTGAAGGTATACTGG | 66 | 127 | 171652465 |
| SpMCh13 | ATTGGGACTGGCCACACTGAAGG | 67 | 128 | 171652457 |
| SpMCh14 | GCAGCTGGATTCATTGGGACTGG | 68 | 129 | 171652445 |
| SpMCh15 | TCTGGGCAGCTGGATTCATTGGG | 69 | 130 | 171652440 |
| SpMCh16 | CTCTGGGCAGCTGGATTCATTGG | 70 | 131 | 171652439 |
| SpMCh17 | CTGGCTCTGCTCTGGGCAGCTGG | 71 | 132 | 171652430 |
| SpMCh18 | TAAGTTATGGATGACTGACATGG | 72 | 133 | 171652406 |
| SpMCh19 | CAGCACCCAACGCTTAGACCTGG | 73 | 134 | 171652361 |
| SpMCh20 | CACCCAACGCTTAGACCTGGAGG | 74 | 135 | 171652358 |
| SpMCh21 | GGCCTCCAGGTCTAAGCGTTGGG | 75 | 136 | 171652367 |
| SpMCh22 | TGGCCTCCAGGTCTAAGCGTTGG | 76 | 137 | 171652366 |
| SpMCh23 | GTCGAGCTTTGGTGGCCTCCAGG | 77 | 138 | 171652354 |
| SpMCh24 | GGAGCTGAGTCGAGCTTTGGTGG | 78 | 139 | 171652346 |
| SpMCh25 | CAAAGCTCGACTCAGCTCCCTGG | 79 | 140 | 171652331 |
| SpMCh26 | CAGGGAGCTGAGTCGAGCTTTGG | 80 | 141 | 171652343 |
| SpMCh27 | TTGGTGGAGGAGGCTCTCCAGGG | 81 | 142 | 171652325 |
| SpMCh28 | ATTGGTGGAGGAGGCTCTCCAGG | 82 | 143 | 171652324 |
| SpMCh29 | CCTCCTCCACCAATTGACCTTGG | 83 | 144 | 171652304 |
| SpMCh30 | CCAAGGTCAATTGGTGGAGGAGG | 84 | 145 | 171652315 |
| SpMCh31 | CCACCAATTGACCTTGGACCAGG | 85 | 146 | 171652298 |
| SpMCh32 | GGTCCAAGGTCAATTGGTGGAGG | 86 | 147 | 171652312 |
| SpMCh33 | CCTGGTCCAAGGTCAATTGGTGG | 87 | 148 | 171652309 |
| SpMCh34 | CAGCCTGGTCCAAGGTCAATTGG | 88 | 149 | 171652306 |
| SpMCh35 | GGGCACCCTGAGGCGGGAGCGGG | 89 | 150 | 171652232 |
| SpMCh36 | CTGGTCCCGCTCCCGCCTCAGGG | 90 | 151 | 171652238 |
| SpMCh37 | GCTGGTCCCGCTCCCGCCTCAGG | 91 | 152 | 171652237 |
| SpMCh38 | GAGGCGGGAGCGGGACCAGCTGG | 92 | 153 | 171652223 |
| SpMCh39 | CTCTGGTTTGGGTTTCCAGCTGG | 93 | 154 | 171652219 |
| SpMCh40 | AGTCTCCAACTCTCTGGTTTGGG | 94 | 155 | 171652208 |
| SpMCh41 | GTCTCGGAGGAGGTTGCTGTAGG | 95 | 156 | 171652184 |
| SpMCh42 | CCTCCGAGACAAGTCAGTTCTGG | 96 | 157 | 171652160 |
| SpMCh43 | GAACTGACTTGTCTCGGAGGAGG | 97 | 158 | 171652174 |
| SpMCh44 | CCAGAACTGACTTGTCTCGGAGG | 98 | 159 | 171652171 |
| SpMCh45 | CCGAGACAAGTCAGTTCTGGAGG | 99 | 160 | 171652157 |
| SpMCh46 | CCTCCAGAACTGACTTGTCTCGG | 100 | 161 | 171652168 |
| SpMCh47 | AGGAAGAGAAGAAGCGACTAAGG | 101 | 162 | 171652137 |
| SpMCh48 | AAGAAAATGAGAATCTGGCCAGG | 102 | 163 | 171652113 |
| SpMCh49 | TGAGAATCTGGCCAGGAGGTTGG | 103 | 164 | 171652106 |
| SpMCh50 | GCTGCTGCTTTCCAACCTCCTGG | 104 | 165 | 171652106 |
| SpMCh51 | GAGGTTGGAAAGCAGCAGCCAGG | 105 | 166 | 171652091 |
| SpMCh52 | GTTGGAAAGCAGCAGCCAGGAGG | 106 | 167 | 171652088 |
| SpMCh53 | GCAGCAGCCAGGAGGTAGCAAGG | 107 | 168 | 171652080 |
| SpMCh54 | TGTCTCGGGTCTGGGGACACTGG | 108 | 169 | 171652057 |
| SpMCh55 | CCAGACCCGAGACACTGCTCGGG | 109 | 170 | 171652037 |
| SpMCh56 | CGAGCAGTGTCTCGGGTCTGGGG | 110 | 171 | 171652050 |
| SpMCh57 | CCGAGCAGTGTCTCGGGTCTGGG | 111 | 172 | 171652049 |
| SpMCh58 | CCCGAGCAGTGTCTCGGGTCTGG | 112 | 173 | 171652048 |
| SpMCh59 | CACAGCCCGAGCAGTGTCTCGGG | 113 | 174 | 171652043 |
| SpMCh60 | GCACAGCCCGAGCAGTGTCTCGG | 114 | 175 | 171652042 |
| SpMCh61 | ACTGCTCGGGCTGTGCCACCAGG | 115 | 176 | 171652024 |

TABLE 3
DNA Sequences of SpCas9 and SaCas9 tcrRNA sequences used in sgRNAs (in the
tcrRNA sequences, Ts are replaced by Us)

| | | SEQ ID |
|---|---|---|
| Name | tcrRNA sequences | NO |
| Sp tcrRNA | GTTTTAGAGCTAGAAATAGCAAGTTAAAAUAAGGCTAGTCCGTTAT | 177 |
| | CAACTTGAAAAAGTGGCACCGAGTCGGTGCT | |
| Sa tcrRNA | GTTTAAGTACTCTGTGCTGGAAACAGCACAGAATCTACTTAAACAAGGCA | 178 |
| | AAATGCCGTGTTTATCTCGTCAACTTGTTGGCGAGAT | |

TABLE 4
PCR and TIDE Primer Sequences for Analysis of INDEL Frequency at Cut Site
Corresponding to SaCas9 sgRNAs

| | PCR | SEQ ID | PCR | SEQ ID | Sequencing | SEQ | Sequencing | SEQ |
|---|---|---|---|---|---|---|---|---|
| sgRNAs | primer 1 | NO | primer 2 | NO | primer 1 | ID NO | primer 2 | ID NO |
| SaMCh1 | GGGTGCAT | 179 | TACTTGAAG | 180 | CTGTCTTCCC | 181 | CTCTCTGGTT | 182 |
| to | AAATTGGG | | GTGATCGCT | | CCATGAAGGG | | TGGGTTTCCA | |
| SaMCh5 | ATGTTC | | GTGC | | | | GC | |
| SaMCh6 | GGGTGCAT | 179 | TACTTGAAG | 180 | GACAGCTCAG | 183 | GCTGCTGCTT | 184 |
| to | AAATTGGG | | GTGATCGCT | | CTCAGGAAGG | | TCCAACCTCC | |
| SaMCh13 | ATGTTC | | GTGC | | | | | |
| SaMCh12 | GGGTGCAT | 179 | TACTTGAAG | 180 | TACTTGAAGG | 185 | CCAAAGCTCG | 186 |
| to | AAATTGGG | | GTGATCGCT | | TGATCGCTGT | | ACTCAGCTCC | |
| SaMCh13 | ATGTTC | | GTGC | | GC | | | |
| SaMCh14 | CTGCCACC | 187 | TGGGTGATA | 188 | CTGCCACCAC | 187 | TGGGTGATAG | 188 |
| to | ACATCCAG | | GGATAGAGG | | ATCCAGCTAA | | GATAGAGGGC | |
| SaMCh15 | CTAATTC | | GCTTTG | | TTC | | TTTG | |
| SaMCh16 | CGATTTGT | 189 | GTTTGGAGA | 190 | TCTCCAGGGC | 191 | CTTTGGCCTC | 192 |
| to | CTCCAGGG | | GGACAATGG | | TGTCACATCT | | ATCGGTGCTG | |
| SaMCh27 | CTGTC | | CACC | | AC | | | |

TABLE 5
PCR and TIDE Primer Sequences for Analysis of INDEL Frequency at Cut Site
Corresponding to SpCas9 sgRNAs

| | PCR | SEQ ID | PCR | SEQ ID | Sequencing | SEQ ID | Sequencing | SEQ |
|---|---|---|---|---|---|---|---|---|
| sgRNA | primer 1 | NO | primer 2 | NO | primer 1 | NO | primer 2 | ID NO |
| SpMCh1 | GGGTGCAT | 179 | TACTTGAAG | 180 | CTGTCTTCC | 181 | CTCTCTGGTT | 182 |
| to | AAATTGGG | | GTGATCGCT | | CCCATGAAG | | TGGGTTTCCA | |
| SpMCh19 | ATGTTC | | GTGC | | GG | | GC | |
| SpMCh20 | GGGTGCAT | 179 | TACTTGAAG | 180 | GACAGCTCA | 183 | GCTGCTGCTT | 184 |
| to | AAATTGGG | | GTGATCGCT | | GCTCAGGAA | | TCCAACCTCC | |
| SpMCh40 | ATGTTC | | GTGC | | GG | | | |
| SpMCh41 | GGGTGCAT | 179 | TACTTGAAG | 180 | TACTTGAAG | 185 | CCAAAGCTCG | 186 |
| to | AAATTGGG | | GTGATCGCT | | GTGATCGCT | | ACTCAGCTCC | |
| SpMCh61 | ATGTTC | | GTGC | | GTGC | | | |

TABLE 10
Cas9 DNA Sequences

| | | SEQ ID |
|---|---|---|
| Name | Sequence | NO |
| SpCas9 | ATGGGCCCCGCCGCCAAGAGAGTGAAGCTGGACggatccGACAAGAAGTACTCCATTGGGCT | 193 |
| DNA | GGACATTGGCACTAACTCCGTGGGATGGGCCGTGATCACCGACGAGTACAAAGTGCCCAGCA | |
| sequence | AGAAGTTTAAAGTGCTGGGGAATACTGACCGGCACAGCATCAAGAAGAACCTTATAGGCGCC | |
| | CTCCTGTTTGATTCCGGAGAAACCGCTGAAGCCACCCGGCTCAAGAGAACCGCCAGACGCCG | |
| | CTACACCCGGAGGAAGAATCGCATCTGCTATCTGCAAGAGATCTTCTCCAACGAAATGGCCA | |
| | AGGTGGACGACTCGTTCTTCCATCGGCTGGAGGAGTCCTTTCTGGTGGAAGAAGATAAGAAG | |
| | CATGAGAGACACCCCATCTTCGGCAACATCGTGGATGAAGTGGCCTACCACGAAAAGTACCC | |
| | TACCATCTACCACCTTCGCAAGAAGCTCGTGGATAGCACTGATAAGGCGGACCTCCGCCTGA | |
| | TCTACCTCGCGCTCGCCCATATGATCAAGTTCCGGGGACACTTCCTGATCGAGGGGGACCTG | |
| | AACCCTGACAACAGCGACGTGGATAAGCTGTTCATCCAACTGGTGCAAACCTATAACCAGCT | |
| | GTTCGAGGAGAACCCTATCAACGCCTCCGGAGTGGACGCCAAGGCCATCCTGTCGGCTCGCC | |
| | TGTCCAAGTCGAGAAGGCTGGAAAACCTGATTGCCCAGCTCCCGGGAGAAAAGAAGAACGGC | |
| | CTGTTCGGCAACCTGATCGCTCTCTCCCTGGGCCTGACCCCGAATTTCAAGAGCAACTTCGA | |
| | CCTCGCCGAAGATGCAAAGCTCCAGCTGTCAAAAGACACCTACGACGATGACCTGGACAATC | |
| | TGCTGGCACAGATCGGGGATCAGTACGCTGACCTGTTCCTGGCCGCCAAGAACCTGTCCGAC | |
| | GCGATCCTGCTCTCGGATATTCTGAGGGTCAACACCGAGATTACCAAGGCCCCTCTGTCCGC | |
| | GAGCATGATCAAGCGGTACGATGAACATCACCAGGATCTGACACTCTTGAAGGCCCTTGTCC | |
| | GCCAACAACTGCCGGAGAAGTACAAGGAGATTTTCTTTGATCAGTCCAAGAACGGCTACGCT | |
| | GGCTACATTGACGGGGGTGCCAGCCAGGAAGAATTTTACAAGTTCATTAAGCCTATTCTCGA | |
| | AAAGATGGACGGAACTGAGGAGTTGCTCGTGAAGCTGAACCGGGAGGACCTGTTGAGAAAGC | |
| | AACGCACCTTCGACAACGGTTCGATTCCTCATCAAATTCATCTGGGTGAACTGCACGCCATC | |
| | CTCCGGCGGCAGGAGGATTTCTATCCATTCCTGAAAGACAACCGAGAGAAGATTGAGAAAAT | |
| | CCTGACCTTCCGGATACCCTACTACGTGGGACCATTGGCTCGGGGGAACAGCAGATTCGCGT | |
| | GGATGACTAGAAAGTCCGAGGAGACTATTACCCCGTGGAACTTCGAGGAGGTGGTCGATAAG | |
| | GGCGCATCGGCACAGTCCTTCATCGAGCGGATGACCAACTTCGACAAGAACCTTCCCAACGA | |
| | AAAGGTGCTGCCCAAGCACTCGCTGTTGTACGAGTACTTTACCGTGTACAACGAGCTCACTA | |
| | AAGTGAAATACGTGACCGAGGGAATGAGAAAGCCGGCCTTTCTGTCCGGGGAACAGAAGAAG | |
| | GCCATCGTGGACCTCCTCTTCAAAACCAACAGAAAAGTCACCGTGAAGCAGCTGAAGGAGGA | |
| | CTACTTCAAGAAAATCGAGTGCTTCGACTCGGTCGAGATTTCGGGGGTCGAGGATAGGTTTA | |
| | ATGCCAGCCTGGGTACTTACCACGATCTGCTGAAGATCATTAAGGACAAGGACTTCCTTGAC | |
| | AACGAAGAAAACGAGGACATCCTTGAGGACATTGTCCTGACCCTGACCCTGTTTGAGGATCG | |
| | GGAGATGATTGAGGAAAGACTTAAGACCTACGCTCATTTGTTCGACGACAAGGTCATGAAAC | |
| | AGCTGAAGCGGAGGCGGTACACTGGATGGGGTCGGCTGTCCAGGAAGCTGATCAACGGAATC | |
| | CGGGACAAGCAATCCGGAAAGACCATCCTGGACTTCCTGAAGTCAGACGGGTTCGCCAACCG | |
| | GAACTTCATGCAGCTCATTCACGACGACAGCCTGACGTTCAAGGAGGACATCCAGAAGGCAC | |
| | AAGTGTCGGGACAGGGAGACAGCCTCCACGAACACATTGCGAACCTCGCGGGTTCACCGGCT | |
| | ATCAAGAAGGGAATCCTGCAGACTGTGAAGGTGGTGGACGAGTTGGTCAAGGTCATGGGCAG | |
| | GCATAAGCCTGAAAACATCGTGATCGAGATGGCCCGGGAGAACCAGACCACCCAGAAGGGGC | |
| | AGAAGAACAGCAGAGAGCGCATGAAGCGCATTGAGGAGGGCATCAAGGAACTGGGATCACAG | |
| | ATCCTGAAGGAACATCCCGTGGAAAACACGCAGCTGCAGAACGAGAAACTCTACCTGTACTA | |
| | TTTGCAAAACGGCCGCGATATGTACGTGGACCAAGAACTCGATATCAACCGCCTGTCCGACT | |
| | ACGACGTGGACCACATCGTGCCGCAGAGCTTCCTGAAGGATGATTCTATCGATAACAAGGTC | |
| | CTCACCCGGTCGGACAAGAATCGGGGGAAGTCAGATAACGTGCCGTCTGAGGAAGTGGTGAA | |
| | GAAGATGAAGAATTACTGGCGGCAGCTTCTGAACGCGAAACTTATTACCCAGCGGAAATTCG | |
| | ACAACCTGACTAAGGCCGAGCGGGGAGGACTGTCAGAACTGGACAAAGCCGGCTTCATTAAG | |
| | AGACAGCTGGTCGAAACTCGCCAGATCACCAAACATGTGGCCCAGATCCTGGACTCCAGGAT | |
| | GAACACCAAGTACGACGAAAACGATAAGCTCATTCGGGAAGTGAAAGTGATCACACTGAAGT | |
| | CCAAGCTGGTGTCCGACTTCCGCAAGGACTTCCAGTTCTACAAGGTCCGCGAGATTAACAAC | |
| | TACCACCACGCACACGACGCTTACTTGAACGCCGTCGTGGGCACTGCCTTGATTAAGAAATA | |
| | CCCGAAGCTGGAATCCGAGTTCGTGTACGGAGACTACAAGGTGTACGATGTGCGCAAGATGA | |
| | TCGCCAAGTCGGAGCAAGAAATCGGAAAGGCCACCGCTAAGTATTTCTTTTACTCCAACATT | |
| | ATGAACTTCTTCAAGACTGAGATCACCCTGGCCAATGGAGAAATCCGCAAGAGGCCGCTGAT | |
| | CGAAACCAATGGAGAGACTGGAGAGATTGTGTGGGATAAGGGACGCGACTTCGCCACCGTGC | |
| | GCAAGGTGCTGAGCATGCCCCAAGTCAACATTGTGAAAAAGACCGAAGTGCAGACGGGCGGT | |
| | TTCTCAAAGGAAAGCATCCTGCCTAAGCGGAACTCCGATAAGCTGATCGCGCGCAAGAAGGA | |
| | CTGGGACCCGAAGAAATATGGCGGCTTCGACTCCCCCACCGTCGCCTACTCGGTGCTCGTCG | |
| | TGGCTAAAGTGGAGAAGGGAAAGTCGAAGAAGCTCAAGTCCGTGAAGGAATTGCTGGGTATT | |
| | ACTATTATGGAACGGTCCAGCTTCGAGAAGAATCCGATCGACTTCCTGGAGGCCAAGGGATA | |
| | CAAGGAAGTGAAGAAGGACCTGATCATTAAGCTGCCGAAGTACAGCCTTTTTGAGCTGGAAA | |
| | ACGGACGCAAGCGGATGCTGGCCTCCGCCGGAGAGCTGCAGAAGGGCAACGAACTGGCCCTC | |
| | CCGTCCAAATACGTGAACTTTCTGTACCTGGCCAGCCACTACGAGAAGCTGAAGGGATCACC | |
| | TGAAGATAACGAGCAGAAGCAGCTGTTCGTGGAACAACATAAGCATTATCTTGACGAGATCA | |
| | TTGAACAGATCTCTGAGTTCTCCAAGAGAGTGATTCTGGCTGACGCTAACCTTGACAAAGTG | |
| | CTGAGCGCTTACAACAAGCACAGGGACAAGCCCATCCGGGAGCAGGCAGAGAACATCATTCA | |
| | CCTGTTCACTCTCACCAACTTGGGTGCCCCGGCAGCCTTCAAGTACTTCGATACCACAATCG | |
| | ACCGCAAGAGGTACACCTCAACCAAGGAGGTCCTTGACGCTACCCTGATCCATCAATCCATT | |
| | ACCGGCCTGTACGAAACTAGGATCGACCTGTCGCAGCTGGGTGGCGACAAGCTTCCTGCCGC | |
| | CAAGAGAGTGAAGCTGGACtaa | |
| SaCas9 | atgCCTGCCGCCAAGAGAGTGAAGCTGGACggatccggaaagcggaactatatcctgggact | 194 |
| DNA | ggacatcggaattacctccgtgggatacggcatcatcgattacgagactagggacgtgattg | |
| sequence | acgccggcgtgagactctttaaggaggccaacgtggaaaacaacgaaggtcgcagatccaag | |
| | cggggtgcaagacgcctgaagcgccggaggagacatcggatacagcgcgtgaagaagctcct | |
| | tttcgactacaacctcctcactgaccactcggaattgtccggtatcaacccctacgaagccc | |
| | gcgtgaaaggcctgagccagaagctgtccgaagaggagtttagcgcagccctgctgcacctg | |
| | gctaagcgaaggggggtgcacaacgtgaacgaggtggaggaggacactggcaacgaactgtc | |
| | caccaaggagcagatttcacggaactcgaaggcgctggaagagaaatatgtggccgagctgc | |
| | agctggagaggctcaagaaggatggcgaagtccgggggagcatcaatcgcttcaagacctcg | |
| | gactacgtgaaggaagccaaacagctgttgaaggtgcagaaggcctaccaccaactggacca | |
| | atcattcattgacacttacatcgatctgcttgaaaccaggcgcacctactacgagggtcctg | |
| | gagaaggcagccctttcggatggaaggacatcaaggagtggtatgagatgctgatgggtcat | |
| | tgcacctactttccggaagaactgcgctcagtgaagtacgcgtacaacgctgacctctacaa | |
| | cgctctcaacgatctgaacaacctcgtgatcacccgggacgagaacgaaaagctggagtact | |
| | acgaaaagttccagattatcgaaaacgtgttcaagcagaagaagaagcccaccctgaagcag | |
| | attgcaaaggagatccttgtgaacgaggaggatattaagggctaccgggtcacctccaccgg | |
| | gaaaccagagttcactaatctcaaggtgtaccatgacattaaggacattactgcccgcaagg | |
| | agatcattgaaaacgcggaactgctggaccaaatcgcgaagatcctgaccatctatcagagc | |
| | tccgaggatatccaggaggaacttactaacctcaattccgagctgacgcaggaagaaatcga | |
| | gcaaattagcaacctgaagggttacactggaacccacaacctcagcttgaaagcgattaacc | |
| | ttattttggatgaactttggcacactaatgacaatcagatcgccattttcaaccggctgaaa | |
| | ctggtgccgaagaaggtggacctgagccaacagaaggaaatcccgaccacccttgtggacga | |
| | tttcatcctgtcacctgtggtgaagaggagcttcatccagtcgatcaaggtcatcaacgcca | |
| | tcataaagaagtacggccttcccaacgacatcatcatcgaactggcccgcgagaagaactcc | |
| | aaagatgcccagaagatgatcaacgagatgcagaagcgaaaccggcagacgaacgaacggat | |
| | cgaggagatcatccggaccaccgggaaggaaaacgcgaagtacctgatcgagaaaatcaagc | |
| | tgcatgatatgcaggaagggaagtgtctctactccctggaggccattccgctggaggatttg | |
| | ctgaacaaccctttcaactacgaagtcgatcatatcattcctcgctccgtgtccttcgataa | |
| | ctccttcaacaataaggtcctcgtgaagcaggaggagaactcgaagaagggcaacagaaccc | |
| | cgttccagtacctctcgtcgtccgactccaagatcagctacgaaactttcaagaagcacatt | |
| | ctgaacctggccaagggcaaagggagaattagcaagaccaagaaggaatacctcctggaaga | |
| | gagagacatcaaccgcttctcggtgcaaaaggatttcatcaaccgcaacctggtcgatacca | |
| | gatacgccaccaggggactgatgaacctcctgcggtcctacttccgggtcaacaatctggac | |
| | gtgaaggtcaaatccatcaacgggggctttacttctttcctgcgccggaagtggaagttcaa | |
| | gaaggaacggaacaagggatacaagcaccacgctgaagatgccctgattattgccaacgccg | |
| | acttcatctttaaggaatggaaaaagctggacaaggctaagaaggtcatggagaaccagatg | |
| | ttcgaagaaaagcaggccgagtccatgcccgaaatcgaaaccgagcaggaatacaaggagat | |
| | cttcatcacaccgcaccaaatcaagcacatcaaggacttcaaggattacaagtacagccacc | |
| | gggtggacaagaagcctaacagagagcttatcaacgacaccctgtactccacgcgcaaggac | |
| | gacaagggaaacacattgatcgtgaacaacctgaacggactgtatgacaaggacaatgacaa | |
| | actgaagaagctgatcaacaaatcgccggaaaagctcctgatgtaccatcacgaccctcaaa | |
| | cctaccagaaactgaagctcatcatggagcagtacggcgacgaaaagaatcccctgtacaaa | |
| | tactacgaggagactggaaattacctgactaagtactccaagaaggataacggccccgtgat | |
| | caagaagattaagtactacggaaacaaactgaacgcacatctcgacatcaccgatgattatc | |
| | caaactcccgcaacaaagtcgtgaagctctccctcaaaccgtaccgcttcgacgtgtacctg | |
| | gataatggggtgtacaagttcgtgaccgtgaagaacctggacgtcattaagaaggaaaacta | |
| | ctacgaagtgaactcaaagtgctacgaggaagccaagaagctcaagaagatcagcaaccagg | |
| | ccgagttcatcgcatcgttttacaacaatgacctcattaagattaatggagaactgtacaga | |
| | gtgatcggcgtgaacaacgacctcctgaaccggattgaagtgaacatgatcgatattaccta | |
| | ccgggagtatctggagaacatgaacgacaagcgcccaccgagaatcatcaaaactattgcct | |
| | ccaagacccaatccattaagaaatactccaccgacatcctgggcaacctgtacgaggtcaag | |
| | tcgaagaagcacccccagattatcaagaagggaaagcttCCTGCCGCCAAGAGAGTGAAGCT | |
| | GGACtaa | |

TABLE 11
SaCas9 sgRNA Unmodified Sequences for SaCas9 sgRNAs Targeting the MYOC
Coding sequence

| | | SEQ |
|---|---|---|
| | | ID |
| Name | sgRNA sequence | NO |
| SaMCh1 | CUGCUUCUGGCCUGCCUGGUGGUUUUAGUACUCUGGAAACAGAAUCUACUAAAACAAGGCAAAAUG | 195 |
| | CCGUGUUUAUCUCGUCAACUUGUUGGCGAGAU | |
| SaMCh2 | CAGCUCAGGAAGGCCAAUGACGUUUUAGUACUCUGGAAACAGAAUCUACUAAAACAAGGCAAAAUG | 196 |
| | CCGUGUUUAUCUCGUCAACUUGUUGGCGAGAU | |
| SaMCh3 | CCUUCAGUGUGGCCAGUCCCAGUUUUAGUACUCUGGAAACAGAAUCUACUAAAACAAGGCAAAAUG | 197 |
| | CCGUGUUUAUCUCGUCAACUUGUUGGCGAGAU | |
| SaMCh4 | GCCUGGCUCUGCUCUGGGCAGGUUUUAGUACUCUGGAAACAGAAUCUACUAAAACAAGGCAAAAUG | 198 |
| | CCGUGUUUAUCUCGUCAACUUGUUGGCGAGAU | |
| SaMCh5 | GCUGCUGUCUCUCUGUAAGUUGUUUUAGUACUCUGGAAACAGAAUCUACUAAAACAAGGCAAAAUG | 199 |
| | CCGUGUUUAUCUCGUCAACUUGUUGGCGAGAU | |
| SaMCh6 | GGUGGCCUCCAGGUCUAAGCGGUUUUAGUACUCUGGAAACAGAAUCUACUAAAACAAGGCAAAAUG | 200 |
| | CCGUGUUUAUCUCGUCAACUUGUUGGCGAGAU | |
| SaMCh7 | UGGAGGAGGCUCUCCAGGGAGGUUUUAGUACUCUGGAAACAGAAUCUACUAAAACAAGGCAAAAUG | 201 |
| | CCGUGUUUAUCUCGUCAACUUGUUGGCGAGAU | |
| SaMCh8 | CAGCUCCCUCUGCAGCCCCUCGUUUUAGUACUCUGGAAACAGAAUCUACUAAAACAAGGCAAAAUG | 202 |
| | CCGUGUUUAUCUCGUCAACUUGUUGGCGAGAU | |
| SaMCh9 | CAGCUGGUCCCGCUCCCGCCUGUUUUAGUACUCUGGAAACAGAAUCUACUAAAACAAGGCAAAAUG | 203 |
| | CCGUGUUUAUCUCGUCAACUUGUUGGCGAGAU | |
| SaMCh10 | ACCAGCUGGAAACCCAAACCAGUUUUAGUACUCUGGAAACAGAAUCUACUAAAACAAGGCAAAAUG | 204 |
| | CCGUGUUUAUCUCGUCAACUUGUUGGCGAGAU | |
| SaMCh11 | GGCAGUCUCCAACUCUCUGGUGUUUUAGUACUCUGGAAACAGAAUCUACUAAAACAAGGCAAAAUG | 205 |
| | CCGUGUUUAUCUCGUCAACUUGUUGGCGAGAU | |
| SaMCh12 | AAGCGACUAAGGCAAGAAAAUGUUUUAGUACUCUGGAAACAGAAUCUACUAAAACAAGGCAAAAUG | 206 |
| | CCGUGUUUAUCUCGUCAACUUGUUGGCGAGAU | |
| SaMCh13 | UGGCACAGCCCGAGCAGUGUCGUUUUAGUACUCUGGAAACAGAAUCUACUAAAACAAGGCAAAAUG | 207 |
| | CCGUGUUUAUCUCGUCAACUUGUUGGCGAGAU | |
| SaMCh14 | GCUAACUGAAGUUCCUGCUUCGUUUUAGUACUCUGGAAACAGAAUCUACUAAAACAAGGCAAAAUG | 208 |
| | CCGUGUUUAUCUCGUCAACUUGUUGGCGAGAU | |
| SaMCh15 | GAGAGCCCAUCUGGCUAUCUCGUUUUAGUACUCUGGAAACAGAAUCUACUAAAACAAGGCAAAAUG | 209 |
| | CCGUGUUUAUCUCGUCAACUUGUUGGCGAGAU | |
| SaMCh16 | AAUUACUGGCAAGUAUGGUGUGUUUUAGUACUCUGGAAACAGAAUCUACUAAAACAAGGCAAAAUG | 210 |
| | CCGUGUUUAUCUCGUCAACUUGUUGGCGAGAU | |
| SaMCh17 | GGGUGUAGGGGUAGGUGGGCUGUUUUAGUACUCUGGAAACAGAAUCUACUAAAACAAGGCAAAAUG | 211 |
| | CCGUGUUUAUCUCGUCAACUUGUUGGCGAGAU | |
| SaMCh18 | CUACACCCAGGAGACCACGUGGUUUUAGUACUCUGGAAACAGAAUCUACUAAAACAAGGCAAAAUG | 212 |
| | CCGUGUUUAUCUCGUCAACUUGUUGGCGAGAU | |
| SaMCh19 | UCCACGUGGUCUCCUGGGUGUGUUUUAGUACUCUGGAAACAGAAUCUACUAAAACAAGGCAAAAUG | 213 |
| | CCGUGUUUAUCUCGUCAACUUGUUGGCGAGAU | |
| SaMCh20 | GUCGAUUCUCCACGUGGUCUCGUUUUAGUACUCUGGAAACAGAAUCUACUAAAACAAGGCAAAAUG | 214 |
| | CCGUGUUUAUCUCGUCAACUUGUUGGCGAGAU | |
| SaMCh21 | UGGAGAAUCGACACAGUUGGCGUUUUAGUACUCUGGAAACAGAAUCUACUAAAACAAGGCAAAAUG | 215 |
| | CCGUGUUUAUCUCGUCAACUUGUUGGCGAGAU | |
| SaMCh22 | GCACGGAUGUCCGCCAGGUUUGUUUUAGUACUCUGGAAACAGAAUCUACUAAAACAAGGCAAAAUG | 216 |
| | CCGUGUUUAUCUCGUCAACUUGUUGGCGAGAU | |
| SaMCh23 | UAGGCAGUAUGUGAACCUUAGGUUUUAGUACUCUGGAAACAGAAUCUACUAAAACAAGGCAAAAUG | 217 |
| | CCGUGUUUAUCUCGUCAACUUGUUGGCGAGAU | |
| SaMCh24 | CUGCCUAGGCCACUGGAAAGCGUUUUAGUACUCUGGAAACAGAAUCUACUAAAACAAGGCAAAAUG | 218 |
| | CCGUGUUUAUCUCGUCAACUUGUUGGCGAGAU | |
| SaMCh25 | GGAGCCUCUAUUUCCAGGGCGGUUUUAGUACUCUGGAAACAGAAUCUACUAAAACAAGGCAAAAUG | 219 |
| | CCGUGUUUAUCUCGUCAACUUGUUGGCGAGAU | |
| SaMCh26 | CGCCCUGGAAAUAGAGGCUCCGUUUUAGUACUCUGGAAACAGAAUCUACUAAAACAAGGCAAAAUG | 220 |
| | CCGUGUUUAUCUCGUCAACUUGUUGGCGAGAU | |
| SaMCh27 | AGAACUGUCAUAAGAUAUGAGGUUUUAGUACUCUGGAAACAGAAUCUACUAAAACAAGGCAAAAUG | 221 |
| | CCGUGUUUAUCUCGUCAACUUGUUGGCGAGAU | |

TABLE 12
SaCas9 sgRNA Modified Sequences for SaCas9 sgRNAs Targeting the MYOC
Coding sequence (screening/small batches)

| | | SEQ |
|---|---|---|
| | gRNA sequence | ID |
| Name | (Modified nucleotides in bold) | NO |
| SaMCh1 | csusgsCUUCUGGCCUGCCUGGUGGUUUUAGUACUCUGGAAACAGAAUCUACUAAAACAAGGCAA | 222 |
| | AAUGCCGUGUUUAUCUCGUCAACUUGUUGGCGAGAusususu | |
| SaMCh2 | csasgsCUCAGGAAGGCCAAUGACGUUUUAGUACUCUGGAAACAGAAUCUACUAAAACAAGGCAA | 223 |
| | AAUGCCGUGUUUAUCUCGUCAACUUGUUGGCGAGAusususu | |
| SaMCh3 | cscsusUCAGUGUGGCCAGUCCCAGUUUUAGUACUCUGGAAACAGAAUCUACUAAAACAAGGCAA | 224 |
| | AAUGCCGUGUUUAUCUCGUCAACUUGUUGGCGAGAusususu | |
| SaMCh4 | gscscsUGGCUCUGCUCUGGGCAGGUUUUAGUACUCUGGAAACAGAAUCUACUAAAACAAGGCAA | 225 |
| | AAUGCCGUGUUUAUCUCGUCAACUUGUUGGCGAGAusususu | |
| SaMCh5 | gscsusGCUGUCUCUCUGUAAGUUGUUUUAGUACUCUGGAAACAGAAUCUACUAAAACAAGGCAA | 226 |
| | AAUGCCGUGUUUAUCUCGUCAACUUGUUGGCGAGAusususu | |
| SaMCh6 | gsgsusGGCCUCCAGGUCUAAGCGGUUUUAGUACUCUGGAAACAGAAUCUACUAAAACAAGGCAA | 227 |
| | AAUGCCGUGUUUAUCUCGUCAACUUGUUGGCGAGAusususu | |
| SaMCh7 | usgsgsAGGAGGCUCUCCAGGGAGGUUUUAGUACUCUGGAAACAGAAUCUACUAAAACAAGGCAA | 228 |
| | AAUGCCGUGUUUAUCUCGUCAACUUGUUGGCGAGAusususu | |
| SaMCh8 | csasgsCUCCCUCUGCAGCCCCUCGUUUUAGUACUCUGGAAACAGAAUCUACUAAAACAAGGCAA | 229 |
| | AAUGCCGUGUUUAUCUCGUCAACUUGUUGGCGAGAusususu | |
| SaMCh9 | csasgsCUGGUCCCGCUCCCGCCUGUUUUAGUACUCUGGAAACAGAAUCUACUAAAACAAGGCAA | 230 |
| | AAUGCCGUGUUUAUCUCGUCAACUUGUUGGCGAGAusususu | |
| SaMCh10 | ascscsAGCUGGAAACCCAAACCAGUUUUAGUACUCUGGAAACAGAAUCUACUAAAACAAGGCAA | 231 |
| | AAUGCCGUGUUUAUCUCGUCAACUUGUUGGCGAGAusususu | |
| SaMCh11 | gsgscsAGUCUCCAACUCUCUGGUGUUUUAGUACUCUGGAAACAGAAUCUACUAAAACAAGGCAA | 232 |
| | AAUGCCGUGUUUAUCUCGUCAACUUGUUGGCGAGAusususu | |
| SaMCh12 | asasgsCGACUAAGGCAAGAAAAUGUUUUAGUACUCUGGAAACAGAAUCUACUAAAACAAGGCAA | 233 |
| | AAUGCCGUGUUUAUCUCGUCAACUUGUUGGCGAGAusususu | |
| SaMCh13 | usgsgsCACAGCCCGAGCAGUGUCGUUUUAGUACUCUGGAAACAGAAUCUACUAAAACAAGGCAA | 234 |
| | AAUGCCGUGUUUAUCUCGUCAACUUGUUGGCGAGAusususu | |
| SaMCh14 | gscsusAACUGAAGUUCCUGCUUCGUUUUAGUACUCUGGAAACAGAAUCUACUAAAACAAGGCAA | 235 |
| | AAUGCCGUGUUUAUCUCGUCAACUUGUUGGCGAGAusususu | |
| SaMCh15 | gsasgsAGCCCAUCUGGCUAUCUCGUUUUAGUACUCUGGAAACAGAAUCUACUAAAACAAGGCAA | 236 |
| | AAUGCCGUGUUUAUCUCGUCAACUUGUUGGCGAGAusususu | |
| SaMCh16 | asasusUACUGGCAAGUAUGGUGUGUUUUAGUACUCUGGAAACAGAAUCUACUAAAACAAGGCAA | 237 |
| | AAUGCCGUGUUUAUCUCGUCAACUUGUUGGCGAGAusususu | |
| SaMCh17 | gsgsgsUGUAGGGGUAGGUGGGCUGUUUUAGUACUCUGGAAACAGAAUCUACUAAAACAAGGCAA | 238 |
| | AAUGCCGUGUUUAUCUCGUCAACUUGUUGGCGAGAusususu | |
| SaMCh18 | csusasCACCCAGGAGACCACGUGGUUUUAGUACUCUGGAAACAGAAUCUACUAAAACAAGGCAA | 239 |
| | AAUGCCGUGUUUAUCUCGUCAACUUGUUGGCGAGAusususu | |
| SaMCh19 | uscscsACGUGGUCUCCUGGGUGUGUUUUAGUACUCUGGAAACAGAAUCUACUAAAACAAGGCAA | 240 |
| | AAUGCCGUGUUUAUCUCGUCAACUUGUUGGCGAGAusususu | |
| SaMCh20 | gsuscsGAUUCUCCACGUGGUCUCGUUUUAGUACUCUGGAAACAGAAUCUACUAAAACAAGGCAA | 241 |
| | AAUGCCGUGUUUAUCUCGUCAACUUGUUGGCGAGAusususu | |
| SaMCh21 | usgsgsAGAAUCGACACAGUUGGCGUUUUAGUACUCUGGAAACAGAAUCUACUAAAACAAGGCAA | 242 |
| | AAUGCCGUGUUUAUCUCGUCAACUUGUUGGCGAGAusususu | |
| SaMCh22 | gscsasCGGAUGUCCGCCAGGUUUGUUUUAGUACUCUGGAAACAGAAUCUACUAAAACAAGGCAA | 243 |
| | AAUGCCGUGUUUAUCUCGUCAACUUGUUGGCGAGAusususu | |
| SaMCh23 | usasgsGCAGUAUGUGAACCUUAGGUUUUAGUACUCUGGAAACAGAAUCUACUAAAACAAGGCAA | 244 |
| | AAUGCCGUGUUUAUCUCGUCAACUUGUUGGCGAGAusususu | |
| SaMCh24 | csusgsCCUAGGCCACUGGAAAGCGUUUUAGUACUCUGGAAACAGAAUCUACUAAAACAAGGCAA | 245 |
| | AAUGCCGUGUUUAUCUCGUCAACUUGUUGGCGAGAusususu | |
| SaMCh25 | gsgsasGCCUCUAUUUCCAGGGCGGUUUUAGUACUCUGGAAACAGAAUCUACUAAAACAAGGCAA | 246 |
| | AAUGCCGUGUUUAUCUCGUCAACUUGUUGGCGAGAusususu | |
| SaMCh26 | csgscsCCUGGAAAUAGAGGCUCCGUUUUAGUACUCUGGAAACAGAAUCUACUAAAACAAGGCAA | 247 |
| | AAUGCCGUGUUUAUCUCGUCAACUUGUUGGCGAGAusususu | |
| SaMCh27 | asgsasACUGUCAUAAGAUAUGAGGUUUUAGUACUCUGGAAACAGAAUCUACUAAAACAAGGCAA | 248 |
| | AAUGCCGUGUUUAUCUCGUCAACUUGUUGGCGAGAusususu | |

A, G, U, C, N: RNA nucleotide
a, g, u, c, n: 2′-O-Methyl-nucleotide
s: Phosphorothioate linkage

TABLE 13
SpCas9 sgRNA Unmodified Sequences for SpCas9 sgRNAs Targeting the
MYOC Coding sequence

| | | SEQ |
|---|---|---|
| Name | Guide RNA | ID NO |
| SpMCh1 | GUGCACGUUGCUGCAGCUUUGUUUUAGAGCUAGAAAUAGCAAGUUAAAAUAAGGCUAGUCCGUU | 249 |
| | AUCAACUUGAAAAAGUGGCACCGAGUCGGUGCU | |
| SpMCh2 | AGCUGGACAGCUGGCAUCUCGUUUUAGAGCUAGAAAUAGCAAGUUAAAAUAAGGCUAGUCCGUU | 250 |
| | AUCAACUUGAAAAAGUGGCACCGAGUCGGUGCU | |
| SpMCh3 | AGCUGUCCAGCUGCUGCUUCGUUUUAGAGCUAGAAAUAGCAAGUUAAAAUAAGGCUAGUCCGUU | 251 |
| | AUCAACUUGAAAAAGUGGCACCGAGUCGGUGCU | |
| SpMCh4 | GCUUCUGGCCUGCCUGGUGUGUUUUAGAGCUAGAAAUAGCAAGUUAAAAUAAGGCUAGUCCGUU | 252 |
| | AUCAACUUGAAAAAGUGGCACCGAGUCGGUGCU | |
| SpMCh5 | GCCUGCCUGGUGUGGGAUGUGUUUUAGAGCUAGAAAUAGCAAGUUAAAAUAAGGCUAGUCCGUU | 253 |
| | AUCAACUUGAAAAAGUGGCACCGAGUCGGUGCU | |
| SpMCh6 | CUGCCUGGUGUGGGAUGUGGGUUUUAGAGCUAGAAAUAGCAAGUUAAAAUAAGGCUAGUCCGUU | 254 |
| | AUCAACUUGAAAAAGUGGCACCGAGUCGGUGCU | |
| SpMCh7 | GGGCCAGGACAGCUCAGCUCGUUUUAGAGCUAGAAAUAGCAAGUUAAAAUAAGGCUAGUCCGUU | 255 |
| | AUCAACUUGAAAAAGUGGCACCGAGUCGGUGCU | |
| SpMCh8 | GCAUCGGCCACUCUGGUCAUGUUUUAGAGCUAGAAAUAGCAAGUUAAAAUAAGGCUAGUCCGUU | 256 |
| | AUCAACUUGAAAAAGUGGCACCGAGUCGGUGCU | |
| SpMCh9 | UAUACUGGCAUCGGCCACUCGUUUUAGAGCUAGAAAUAGCAAGUUAAAAUAAGGCUAGUCCGUU | 257 |
| | AUCAACUUGAAAAAGUGGCACCGAGUCGGUGCU | |
| SpMCh10 | AUGCCAGUAUACCUUCAGUGGUUUUAGAGCUAGAAAUAGCAAGUUAAAAUAAGGCUAGUCCGUU | 258 |
| | AUCAACUUGAAAAAGUGGCACCGAGUCGGUGCU | |
| SpMCh11 | CACUGAAGGUAUACUGGCAUGUUUUAGAGCUAGAAAUAGCAAGUUAAAAUAAGGCUAGUCCGUU | 259 |
| | AUCAACUUGAAAAAGUGGCACCGAGUCGGUGCU | |
| SpMCh12 | UGGCCACACUGAAGGUAUACGUUUUAGAGCUAGAAAUAGCAAGUUAAAAUAAGGCUAGUCCGUU | 260 |
| | AUCAACUUGAAAAAGUGGCACCGAGUCGGUGCU | |
| SpMCh13 | AUUGGGACUGGCCACACUGAGUUUUAGAGCUAGAAAUAGCAAGUUAAAAUAAGGCUAGUCCGUU | 261 |
| | AUCAACUUGAAAAAGUGGCACCGAGUCGGUGCU | |
| SpMCh14 | GCAGCUGGAUUCAUUGGGACGUUUUAGAGCUAGAAAUAGCAAGUUAAAAUAAGGCUAGUCCGUU | 262 |
| | AUCAACUUGAAAAAGUGGCACCGAGUCGGUGCU | |
| SpMCh15 | UCUGGGCAGCUGGAUUCAUUGUUUUAGAGCUAGAAAUAGCAAGUUAAAAUAAGGCUAGUCCGUU | 263 |
| | AUCAACUUGAAAAAGUGGCACCGAGUCGGUGCU | |
| SpMCh16 | CUCUGGGCAGCUGGAUUCAUGUUUUAGAGCUAGAAAUAGCAAGUUAAAAUAAGGCUAGUCCGUU | 264 |
| | AUCAACUUGAAAAAGUGGCACCGAGUCGGUGCU | |
| SpMCh17 | CUGGCUCUGCUCUGGGCAGCGUUUUAGAGCUAGAAAUAGCAAGUUAAAAUAAGGCUAGUCCGUU | 265 |
| | AUCAACUUGAAAAAGUGGCACCGAGUCGGUGCU | |
| SpMCh18 | UAAGUUAUGGAUGACUGACAGUUUUAGAGCUAGAAAUAGCAAGUUAAAAUAAGGCUAGUCCGUU | 266 |
| | AUCAACUUGAAAAAGUGGCACCGAGUCGGUGCU | |
| SpMCh19 | CAGCACCCAACGCUUAGACCGUUUUAGAGCUAGAAAUAGCAAGUUAAAAUAAGGCUAGUCCGUU | 267 |
| | AUCAACUUGAAAAAGUGGCACCGAGUCGGUGCU | |
| SpMCh20 | CACCCAACGCUUAGACCUGGGUUUUAGAGCUAGAAAUAGCAAGUUAAAAUAAGGCUAGUCCGUU | 268 |
| | AUCAACUUGAAAAAGUGGCACCGAGUCGGUGCU | |
| SpMCh21 | GGCCUCCAGGUCUAAGCGUUGUUUUAGAGCUAGAAAUAGCAAGUUAAAAUAAGGCUAGUCCGUU | 269 |
| | AUCAACUUGAAAAAGUGGCACCGAGUCGGUGCU | |
| SpMCh22 | UGGCCUCCAGGUCUAAGCGUGUUUUAGAGCUAGAAAUAGCAAGUUAAAAUAAGGCUAGUCCGUU | 270 |
| | AUCAACUUGAAAAAGUGGCACCGAGUCGGUGCU | |
| SpMCh23 | GUCGAGCUUUGGUGGCCUCCGUUUUAGAGCUAGAAAUAGCAAGUUAAAAUAAGGCUAGUCCGUU | 271 |
| | AUCAACUUGAAAAAGUGGCACCGAGUCGGUGCU | |
| SpMCh24 | GGAGCUGAGUCGAGCUUUGGGUUUUAGAGCUAGAAAUAGCAAGUUAAAAUAAGGCUAGUCCGUU | 272 |
| | AUCAACUUGAAAAAGUGGCACCGAGUCGGUGCU | |
| SpMCh25 | CAAAGCUCGACUCAGCUCCCGUUUUAGAGCUAGAAAUAGCAAGUUAAAAUAAGGCUAGUCCGUU | 273 |
| | AUCAACUUGAAAAAGUGGCACCGAGUCGGUGCU | |
| SpMCh26 | CAGGGAGCUGAGUCGAGCUUGUUUUAGAGCUAGAAAUAGCAAGUUAAAAUAAGGCUAGUCCGUU | 274 |
| | AUCAACUUGAAAAAGUGGCACCGAGUCGGUGCU | |
| SpMCh27 | UUGGUGGAGGAGGCUCUCCAGUUUUAGAGCUAGAAAUAGCAAGUUAAAAUAAGGCUAGUCCGUU | 275 |
| | AUCAACUUGAAAAAGUGGCACCGAGUCGGUGCU | |
| SpMCh28 | AUUGGUGGAGGAGGCUCUCCGUUUUAGAGCUAGAAAUAGCAAGUUAAAAUAAGGCUAGUCCGUU | 276 |
| | AUCAACUUGAAAAAGUGGCACCGAGUCGGUGCU | |
| SpMCh29 | CCUCCUCCACCAAUUGACCUGUUUUAGAGCUAGAAAUAGCAAGUUAAAAUAAGGCUAGUCCGUU | 277 |
| | AUCAACUUGAAAAAGUGGCACCGAGUCGGUGCU | |
| SpMCh30 | CCAAGGUCAAUUGGUGGAGGGUUUUAGAGCUAGAAAUAGCAAGUUAAAAUAAGGCUAGUCCGUU | 278 |
| | AUCAACUUGAAAAAGUGGCACCGAGUCGGUGCU | |
| SpMCh31 | CCACCAAUUGACCUUGGACCGUUUUAGAGCUAGAAAUAGCAAGUUAAAAUAAGGCUAGUCCGUU | 279 |
| | AUCAACUUGAAAAAGUGGCACCGAGUCGGUGCU | |
| SpMCh32 | GGUCCAAGGUCAAUUGGUGGGUUUUAGAGCUAGAAAUAGCAAGUUAAAAUAAGGCUAGUCCGUU | 280 |
| | AUCAACUUGAAAAAGUGGCACCGAGUCGGUGCU | |
| SpMCh33 | CCUGGUCCAAGGUCAAUUGGGUUUUAGAGCUAGAAAUAGCAAGUUAAAAUAAGGCUAGUCCGUU | 281 |
| | AUCAACUUGAAAAAGUGGCACCGAGUCGGUGCU | |
| SpMCh34 | CAGCCUGGUCCAAGGUCAAUGUUUUAGAGCUAGAAAUAGCAAGUUAAAAUAAGGCUAGUCCGUU | 282 |
| | AUCAACUUGAAAAAGUGGCACCGAGUCGGUGCU | |
| SpMCh35 | GGGCACCCUGAGGCGGGAGCGUUUUAGAGCUAGAAAUAGCAAGUUAAAAUAAGGCUAGUCCGUU | 283 |
| | AUCAACUUGAAAAAGUGGCACCGAGUCGGUGCU | |
| SpMCh36 | CUGGUCCCGCUCCCGCCUCAGUUUUAGAGCUAGAAAUAGCAAGUUAAAAUAAGGCUAGUCCGUU | 284 |
| | AUCAACUUGAAAAAGUGGCACCGAGUCGGUGCU | |
| SpMCh37 | GCUGGUCCCGCUCCCGCCUCGUUUUAGAGCUAGAAAUAGCAAGUUAAAAUAAGGCUAGUCCGUU | 285 |
| | AUCAACUUGAAAAAGUGGCACCGAGUCGGUGCU | |
| SpMCh38 | GAGGCGGGAGCGGGACCAGCGUUUUAGAGCUAGAAAUAGCAAGUUAAAAUAAGGCUAGUCCGUU | 286 |
| | AUCAACUUGAAAAAGUGGCACCGAGUCGGUGCU | |
| SpMCh39 | CUCUGGUUUGGGUUUCCAGCGUUUUAGAGCUAGAAAUAGCAAGUUAAAAUAAGGCUAGUCCGUU | 287 |
| | AUCAACUUGAAAAAGUGGCACCGAGUCGGUGCU | |
| SpMCh40 | AGUCUCCAACUCUCUGGUUUGUUUUAGAGCUAGAAAUAGCAAGUUAAAAUAAGGCUAGUCCGUU | 288 |
| | AUCAACUUGAAAAAGUGGCACCGAGUCGGUGCU | |
| SpMCh41 | GUCUCGGAGGAGGUUGCUGUGUUUUAGAGCUAGAAAUAGCAAGUUAAAAUAAGGCUAGUCCGUU | 289 |
| | AUCAACUUGAAAAAGUGGCACCGAGUCGGUGCU | |
| SpMCh42 | CCUCCGAGACAAGUCAGUUCGUUUUAGAGCUAGAAAUAGCAAGUUAAAAUAAGGCUAGUCCGUU | 290 |
| | AUCAACUUGAAAAAGUGGCACCGAGUCGGUGCU | |
| SpMCh43 | GAACUGACUUGUCUCGGAGGGUUUUAGAGCUAGAAAUAGCAAGUUAAAAUAAGGCUAGUCCGUU | 291 |
| | AUCAACUUGAAAAAGUGGCACCGAGUCGGUGCU | |
| SpMCh44 | CCAGAACUGACUUGUCUCGGGUUUUAGAGCUAGAAAUAGCAAGUUAAAAUAAGGCUAGUCCGUU | 292 |
| | AUCAACUUGAAAAAGUGGCACCGAGUCGGUGCU | |
| SpMCh45 | CCGAGACAAGUCAGUUCUGGGUUUUAGAGCUAGAAAUAGCAAGUUAAAAUAAGGCUAGUCCGUU | 293 |
| | AUCAACUUGAAAAAGUGGCACCGAGUCGGUGCU | |
| SpMCh46 | CCUCCAGAACUGACUUGUCUGUUUUAGAGCUAGAAAUAGCAAGUUAAAAUAAGGCUAGUCCGUU | 294 |
| | AUCAACUUGAAAAAGUGGCACCGAGUCGGUGCU | |
| SpMCh47 | AGGAAGAGAAGAAGCGACUAGUUUUAGAGCUAGAAAUAGCAAGUUAAAAUAAGGCUAGUCCGUU | 295 |
| | AUCAACUUGAAAAAGUGGCACCGAGUCGGUGCU | |
| SpMCh48 | AAGAAAAUGAGAAUCUGGCCGUUUUAGAGCUAGAAAUAGCAAGUUAAAAUAAGGCUAGUCCGUU | 296 |
| | AUCAACUUGAAAAAGUGGCACCGAGUCGGUGCU | |
| SpMCh49 | UGAGAAUCUGGCCAGGAGGUGUUUUAGAGCUAGAAAUAGCAAGUUAAAAUAAGGCUAGUCCGUU | 297 |
| | AUCAACUUGAAAAAGUGGCACCGAGUCGGUGCU | |
| SpMCh50 | GCUGCUGCUUUCCAACCUCCGUUUUAGAGCUAGAAAUAGCAAGUUAAAAUAAGGCUAGUCCGUU | 298 |
| | AUCAACUUGAAAAAGUGGCACCGAGUCGGUGCU | |
| SpMCh51 | GAGGUUGGAAAGCAGCAGCCGUUUUAGAGCUAGAAAUAGCAAGUUAAAAUAAGGCUAGUCCGUU | 299 |
| | AUCAACUUGAAAAAGUGGCACCGAGUCGGUGCU | |
| SpMCh52 | GUUGGAAAGCAGCAGCCAGGGUUUUAGAGCUAGAAAUAGCAAGUUAAAAUAAGGCUAGUCCGUU | 300 |
| | AUCAACUUGAAAAAGUGGCACCGAGUCGGUGCU | |
| SpMCh53 | GCAGCAGCCAGGAGGUAGCAGUUUUAGAGCUAGAAAUAGCAAGUUAAAAUAAGGCUAGUCCGUU | 301 |
| | AUCAACUUGAAAAAGUGGCACCGAGUCGGUGCU | |
| SpMCh54 | UGUCUCGGGUCUGGGGACACGUUUUAGAGCUAGAAAUAGCAAGUUAAAAUAAGGCUAGUCCGUU | 302 |
| | AUCAACUUGAAAAAGUGGCACCGAGUCGGUGCU | |
| SpMCh55 | CCAGACCCGAGACACUGCUCGUUUUAGAGCUAGAAAUAGCAAGUUAAAAUAAGGCUAGUCCGUU | 303 |
| | AUCAACUUGAAAAAGUGGCACCGAGUCGGUGCU | |
| SpMCh56 | CGAGCAGUGUCUCGGGUCUGGUUUUAGAGCUAGAAAUAGCAAGUUAAAAUAAGGCUAGUCCGUU | 304 |
| | AUCAACUUGAAAAAGUGGCACCGAGUCGGUGCU | |
| SpMCh57 | CCGAGCAGUGUCUCGGGUCUGUUUUAGAGCUAGAAAUAGCAAGUUAAAAUAAGGCUAGUCCGUU | 305 |
| | AUCAACUUGAAAAAGUGGCACCGAGUCGGUGCU | |
| SpMCh58 | CCCGAGCAGUGUCUCGGGUCGUUUUAGAGCUAGAAAUAGCAAGUUAAAAUAAGGCUAGUCCGUU | 306 |
| | AUCAACUUGAAAAAGUGGCACCGAGUCGGUGCU | |
| SpMCh59 | CACAGCCCGAGCAGUGUCUCGUUUUAGAGCUAGAAAUAGCAAGUUAAAAUAAGGCUAGUCCGUU | 307 |
| | AUCAACUUGAAAAAGUGGCACCGAGUCGGUGCU | |
| SpMCh60 | GCACAGCCCGAGCAGUGUCUGUUUUAGAGCUAGAAAUAGCAAGUUAAAAUAAGGCUAGUCCGUU | 308 |
| | AUCAACUUGAAAAAGUGGCACCGAGUCGGUGCU | |
| SpMCh61 | ACUGCUCGGGCUGUGCCACCGUUUUAGAGCUAGAAAUAGCAAGUUAAAAUAAGGCUAGUCCGUU | 309 |
| | AUCAACUUGAAAAAGUGGCACCGAGUCGGUGCU | |

TABLE 14
SpCas9 sgRNA modified Sequences for SpCas9 sgRNAs Targeting the MYOC
Coding sequence (screening/small batches)

| | | SEQ |
|---|---|---|
| | sgRNA Sequence | ID |
| Name | (Modified nucleotides in bold) | NO |
| SpMCh1 | gsusgsCACGUUGCUGCAGCUUUGUUUUAGAGCUAGAAAUAGCAAGUUAAAAUAAGGCUAGUCC | 310 |
| | GUUAUCAACUUGAAAAAGUGGCACCGAGUCGGUGCusususu | |
| SpMCh2 | asgscsUGGACAGCUGGCAUCUCGUUUUAGAGCUAGAAAUAGCAAGUUAAAAUAAGGCUAGUCC | 311 |
| | GUUAUCAACUUGAAAAAGUGGCACCGAGUCGGUGCusususu | |
| SpMCh3 | asgscsUGUCCAGCUGCUGCUUCGUUUUAGAGCUAGAAAUAGCAAGUUAAAAUAAGGCUAGUCC | 312 |
| | GUUAUCAACUUGAAAAAGUGGCACCGAGUCGGUGCusususu | |
| SpMCh4 | gscsusUCUGGCCUGCCUGGUGUGUUUUAGAGCUAGAAAUAGCAAGUUAAAAUAAGGCUAGUCC | 313 |
| | GUUAUCAACUUGAAAAAGUGGCACCGAGUCGGUGCusususu | |
| SpMCh5 | gscscsUGCCUGGUGUGGGAUGUGUUUUAGAGCUAGAAAUAGCAAGUUAAAAUAAGGCUAGUCC | 314 |
| | GUUAUCAACUUGAAAAAGUGGCACCGAGUCGGUGCusususu | |
| SpMCh6 | csusgsCCUGGUGUGGGAUGUGGGUUUUAGAGCUAGAAAUAGCAAGUUAAAAUAAGGCUAGUCC | 315 |
| | GUUAUCAACUUGAAAAAGUGGCACCGAGUCGGUGCusususu | |
| SpMCh7 | gsgsgsCCAGGACAGCUCAGCUCGUUUUAGAGCUAGAAAUAGCAAGUUAAAAUAAGGCUAGUCC | 316 |
| | GUUAUCAACUUGAAAAAGUGGCACCGAGUCGGUGCusususu | |
| SpMCh8 | gscsasUCGGCCACUCUGGUCAUGUUUUAGAGCUAGAAAUAGCAAGUUAAAAUAAGGCUAGUCC | 317 |
| | GUUAUCAACUUGAAAAAGUGGCACCGAGUCGGUGCusususu | |
| SpMCh9 | usasusACUGGCAUCGGCCACUCGUUUUAGAGCUAGAAAUAGCAAGUUAAAAUAAGGCUAGUCC | 318 |
| | GUUAUCAACUUGAAAAAGUGGCACCGAGUCGGUGCusususu | |
| SpMCh10 | asusgsCCAGUAUACCUUCAGUGGUUUUAGAGCUAGAAAUAGCAAGUUAAAAUAAGGCUAGUCC | 319 |
| | GUUAUCAACUUGAAAAAGUGGCACCGAGUCGGUGCusususu | |
| SpMCh11 | csascsUGAAGGUAUACUGGCAUGUUUUAGAGCUAGAAAUAGCAAGUUAAAAUAAGGCUAGUCC | 320 |
| | GUUAUCAACUUGAAAAAGUGGCACCGAGUCGGUGCusususu | |
| SpMCh12 | usgsgsCCACACUGAAGGUAUACGUUUUAGAGCUAGAAAUAGCAAGUUAAAAUAAGGCUAGUCC | 321 |
| | GUUAUCAACUUGAAAAAGUGGCACCGAGUCGGUGCusususu | |
| SpMCh13 | asususGGGACUGGCCACACUGAGUUUUAGAGCUAGAAAUAGCAAGUUAAAAUAAGGCUAGUCC | 322 |
| | GUUAUCAACUUGAAAAAGUGGCACCGAGUCGGUGCusususu | |
| SpMCh14 | gscsasGCUGGAUUCAUUGGGACGUUUUAGAGCUAGAAAUAGCAAGUUAAAAUAAGGCUAGUCC | 323 |
| | GUUAUCAACUUGAAAAAGUGGCACCGAGUCGGUGCusususu | |
| SpMCh15 | uscsusGGGCAGCUGGAUUCAUUGUUUUAGAGCUAGAAAUAGCAAGUUAAAAUAAGGCUAGUCC | 324 |
| | GUUAUCAACUUGAAAAAGUGGCACCGAGUCGGUGCusususu | |
| SpMCh16 | csuscsUGGGCAGCUGGAUUCAUGUUUUAGAGCUAGAAAUAGCAAGUUAAAAUAAGGCUAGUCC | 325 |
| | GUUAUCAACUUGAAAAAGUGGCACCGAGUCGGUGCusususu | |
| SpMCh17 | csusgsGCUCUGCUCUGGGCAGCGUUUUAGAGCUAGAAAUAGCAAGUUAAAAUAAGGCUAGUCC | 326 |
| | GUUAUCAACUUGAAAAAGUGGCACCGAGUCGGUGCusususu | |
| SpMCh18 | usasasGUUAUGGAUGACUGACAGUUUUAGAGCUAGAAAUAGCAAGUUAAAAUAAGGCUAGUCC | 327 |
| | GUUAUCAACUUGAAAAAGUGGCACCGAGUCGGUGCusususu | |
| SpMCh19 | csasgsCACCCAACGCUUAGACCGUUUUAGAGCUAGAAAUAGCAAGUUAAAAUAAGGCUAGUCC | 328 |
| | GUUAUCAACUUGAAAAAGUGGCACCGAGUCGGUGCusususu | |
| SpMCh20 | csascsCCAACGCUUAGACCUGGGUUUUAGAGCUAGAAAUAGCAAGUUAAAAUAAGGCUAGUCC | 329 |
| | GUUAUCAACUUGAAAAAGUGGCACCGAGUCGGUGCusususu | |
| SpMCh21 | gsgscsCUCCAGGUCUAAGCGUUGUUUUAGAGCUAGAAAUAGCAAGUUAAAAUAAGGCUAGUCC | 330 |
| | GUUAUCAACUUGAAAAAGUGGCACCGAGUCGGUGCusususu | |
| SpMCh22 | usgsgsCCUCCAGGUCUAAGCGUGUUUUAGAGCUAGAAAUAGCAAGUUAAAAUAAGGCUAGUCC | 331 |
| | GUUAUCAACUUGAAAAAGUGGCACCGAGUCGGUGCusususu | |
| SpMCh23 | gsuscsGAGCUUUGGUGGCCUCCGUUUUAGAGCUAGAAAUAGCAAGUUAAAAUAAGGCUAGUCC | 332 |
| | GUUAUCAACUUGAAAAAGUGGCACCGAGUCGGUGCusususu | |
| SpMCh24 | gsgsasGCUGAGUCGAGCUUUGGGUUUUAGAGCUAGAAAUAGCAAGUUAAAAUAAGGCUAGUCC | 333 |
| | GUUAUCAACUUGAAAAAGUGGCACCGAGUCGGUGCusususu | |
| SpMCh25 | csasasAGCUCGACUCAGCUCCCGUUUUAGAGCUAGAAAUAGCAAGUUAAAAUAAGGCUAGUCC | 334 |
| | GUUAUCAACUUGAAAAAGUGGCACCGAGUCGGUGCusususu | |
| SpMCh26 | csasgsGGAGCUGAGUCGAGCUUGUUUUAGAGCUAGAAAUAGCAAGUUAAAAUAAGGCUAGUCC | 335 |
| | GUUAUCAACUUGAAAAAGUGGCACCGAGUCGGUGCusususu | |
| SpMCh27 | ususgsGUGGAGGAGGCUCUCCAGUUUUAGAGCUAGAAAUAGCAAGUUAAAAUAAGGCUAGUCC | 336 |
| | GUUAUCAACUUGAAAAAGUGGCACCGAGUCGGUGCusususu | |
| SpMCh28 | asususGGUGGAGGAGGCUCUCCGUUUUAGAGCUAGAAAUAGCAAGUUAAAAUAAGGCUAGUCC | 337 |
| | GUUAUCAACUUGAAAAAGUGGCACCGAGUCGGUGCusususu | |
| SpMCh29 | cscsusCCUCCACCAAUUGACCUGUUUUAGAGCUAGAAAUAGCAAGUUAAAAUAAGGCUAGUCC | 338 |
| | GUUAUCAACUUGAAAAAGUGGCACCGAGUCGGUGCusususu | |
| SpMCh30 | cscsasAGGUCAAUUGGUGGAGGGUUUUAGAGCUAGAAAUAGCAAGUUAAAAUAAGGCUAGUCC | 339 |
| | GUUAUCAACUUGAAAAAGUGGCACCGAGUCGGUGCusususu | |
| SpMCh31 | cscsasCCAAUUGACCUUGGACCGUUUUAGAGCUAGAAAUAGCAAGUUAAAAUAAGGCUAGUCC | 340 |
| | GUUAUCAACUUGAAAAAGUGGCACCGAGUCGGUGCusususu | |
| SpMCh32 | gsgsusCCAAGGUCAAUUGGUGGGUUUUAGAGCUAGAAAUAGCAAGUUAAAAUAAGGCUAGUCC | 341 |
| | GUUAUCAACUUGAAAAAGUGGCACCGAGUCGGUGCusususu | |
| SpMCh33 | cscsusGGUCCAAGGUCAAUUGGGUUUUAGAGCUAGAAAUAGCAAGUUAAAAUAAGGCUAGUCC | 342 |
| | GUUAUCAACUUGAAAAAGUGGCACCGAGUCGGUGCusususu | |
| SpMCh34 | csasgsCCUGGUCCAAGGUCAAUGUUUUAGAGCUAGAAAUAGCAAGUUAAAAUAAGGCUAGUCC | 343 |
| | GUUAUCAACUUGAAAAAGUGGCACCGAGUCGGUGCusususu | |
| SpMCh35 | gsgsgsCACCCUGAGGCGGGAGCGUUUUAGAGCUAGAAAUAGCAAGUUAAAAUAAGGCUAGUCC | 344 |
| | GUUAUCAACUUGAAAAAGUGGCACCGAGUCGGUGCusususu | |
| SpMCh36 | csusgsGUCCCGCUCCCGCCUCAGUUUUAGAGCUAGAAAUAGCAAGUUAAAAUAAGGCUAGUCC | 345 |
| | GUUAUCAACUUGAAAAAGUGGCACCGAGUCGGUGCusususu | |
| SpMCh37 | gscsusGGUCCCGCUCCCGCCUCGUUUUAGAGCUAGAAAUAGCAAGUUAAAAUAAGGCUAGUCC | 346 |
| | GUUAUCAACUUGAAAAAGUGGCACCGAGUCGGUGCusususu | |
| SpMCh38 | gsasgsGCGGGAGCGGGACCAGCGUUUUAGAGCUAGAAAUAGCAAGUUAAAAUAAGGCUAGUCC | 347 |
| | GUUAUCAACUUGAAAAAGUGGCACCGAGUCGGUGCusususu | |
| SpMCh39 | csuscsUGGUUUGGGUUUCCAGCGUUUUAGAGCUAGAAAUAGCAAGUUAAAAUAAGGCUAGUCC | 348 |
| | GUUAUCAACUUGAAAAAGUGGCACCGAGUCGGUGCusususu | |
| SpMCh40 | asgsusCUCCAACUCUCUGGUUUGUUUUAGAGCUAGAAAUAGCAAGUUAAAAUAAGGCUAGUCC | 349 |
| | GUUAUCAACUUGAAAAAGUGGCACCGAGUCGGUGCusususu | |
| SpMCh41 | gsuscsUCGGAGGAGGUUGCUGUGUUUUAGAGCUAGAAAUAGCAAGUUAAAAUAAGGCUAGUCC | 350 |
| | GUUAUCAACUUGAAAAAGUGGCACCGAGUCGGUGCusususu | |
| SpMCh42 | cscsusCCGAGACAAGUCAGUUCGUUUUAGAGCUAGAAAUAGCAAGUUAAAAUAAGGCUAGUCC | 351 |
| | GUUAUCAACUUGAAAAAGUGGCACCGAGUCGGUGCusususu | |
| SpMCh43 | gsasasCUGACUUGUCUCGGAGGGUUUUAGAGCUAGAAAUAGCAAGUUAAAAUAAGGCUAGUCC | 352 |
| | GUUAUCAACUUGAAAAAGUGGCACCGAGUCGGUGCusususu | |
| SpMCh44 | cscsasGAACUGACUUGUCUCGGGUUUUAGAGCUAGAAAUAGCAAGUUAAAAUAAGGCUAGUCC | 353 |
| | GUUAUCAACUUGAAAAAGUGGCACCGAGUCGGUGCusususu | |
| SpMCh45 | cscsgsAGACAAGUCAGUUCUGGGUUUUAGAGCUAGAAAUAGCAAGUUAAAAUAAGGCUAGUCC | 354 |
| | GUUAUCAACUUGAAAAAGUGGCACCGAGUCGGUGCusususu | |
| SpMCh46 | cscsusCCAGAACUGACUUGUCUGUUUUAGAGCUAGAAAUAGCAAGUUAAAAUAAGGCUAGUCC | 355 |
| | GUUAUCAACUUGAAAAAGUGGCACCGAGUCGGUGCusususu | |
| SpMCh47 | asgsgsAAGAGAAGAAGCGACUAGUUUUAGAGCUAGAAAUAGCAAGUUAAAAUAAGGCUAGUCC | 356 |
| | GUUAUCAACUUGAAAAAGUGGCACCGAGUCGGUGCusususu | |
| SpMCh48 | asasgsAAAAUGAGAAUCUGGCCGUUUUAGAGCUAGAAAUAGCAAGUUAAAAUAAGGCUAGUCC | 357 |
| | GUUAUCAACUUGAAAAAGUGGCACCGAGUCGGUGCusususu | |
| SpMCh49 | usgsasGAAUCUGGCCAGGAGGUGUUUUAGAGCUAGAAAUAGCAAGUUAAAAUAAGGCUAGUCC | 358 |
| | GUUAUCAACUUGAAAAAGUGGCACCGAGUCGGUGCusususu | |
| SpMCh50 | gscsusGCUGCUUUCCAACCUCCGUUUUAGAGCUAGAAAUAGCAAGUUAAAAUAAGGCUAGUCC | 359 |
| | GUUAUCAACUUGAAAAAGUGGCACCGAGUCGGUGCusususu | |
| SpMCh51 | gsasgsGUUGGAAAGCAGCAGCCGUUUUAGAGCUAGAAAUAGCAAGUUAAAAUAAGGCUAGUCC | 360 |
| | GUUAUCAACUUGAAAAAGUGGCACCGAGUCGGUGCusususu | |
| SpMCh52 | gsususGGAAAGCAGCAGCCAGGGUUUUAGAGCUAGAAAUAGCAAGUUAAAAUAAGGCUAGUCC | 361 |
| | GUUAUCAACUUGAAAAAGUGGCACCGAGUCGGUGCusususu | |
| SpMCh53 | gscsasGCAGCCAGGAGGUAGCAGUUUUAGAGCUAGAAAUAGCAAGUUAAAAUAAGGCUAGUCC | 362 |
| | GUUAUCAACUUGAAAAAGUGGCACCGAGUCGGUGCusususu | |
| SpMCh54 | usgsusCUCGGGUCUGGGGACACGUUUUAGAGCUAGAAAUAGCAAGUUAAAAUAAGGCUAGUCC | 363 |
| | GUUAUCAACUUGAAAAAGUGGCACCGAGUCGGUGCusususu | |
| SpMCh55 | cscsasGACCCGAGACACUGCUCGUUUUAGAGCUAGAAAUAGCAAGUUAAAAUAAGGCUAGUCC | 364 |
| | GUUAUCAACUUGAAAAAGUGGCACCGAGUCGGUGCusususu | |
| SpMCh56 | csgsasGCAGUGUCUCGGGUCUGGUUUUAGAGCUAGAAAUAGCAAGUUAAAAUAAGGCUAGUCC | 365 |
| | GUUAUCAACUUGAAAAAGUGGCACCGAGUCGGUGCusususu | |
| SpMCh57 | cscsgsAGCAGUGUCUCGGGUCUGUUUUAGAGCUAGAAAUAGCAAGUUAAAAUAAGGCUAGUCC | 366 |
| | GUUAUCAACUUGAAAAAGUGGCACCGAGUCGGUGCusususu | |
| SpMCh58 | cscscsGAGCAGUGUCUCGGGUCGUUUUAGAGCUAGAAAUAGCAAGUUAAAAUAAGGCUAGUCC | 367 |
| | GUUAUCAACUUGAAAAAGUGGCACCGAGUCGGUGCusususu | |
| SpMCh59 | csascsAGCCCGAGCAGUGUCUCGUUUUAGAGCUAGAAAUAGCAAGUUAAAAUAAGGCUAGUCC | 368 |
| | GUUAUCAACUUGAAAAAGUGGCACCGAGUCGGUGCusususu | |
| SpMCh60 | gscsasCAGCCCGAGCAGUGUCUGUUUUAGAGCUAGAAAUAGCAAGUUAAAAUAAGGCUAGUCC | 369 |
| | GUUAUCAACUUGAAAAAGUGGCACCGAGUCGGUGCusususu | |
| SpMCh61 | ascsusGCUCGGGCUGUGCCACCGUUUUAGAGCUAGAAAUAGCAAGUUAAAAUAAGGCUAGUCC | 370 |
| | GUUAUCAACUUGAAAAAGUGGCACCGAGUCGGUGCusususu | |

A, G, U, C, N: RNA nucleotide
a, g, u, c, n: 2′-O-Methyl-nucleotide
s: Phosphorothicate linkage

TABLE 15
SpCas9 sgRNA modified Sequences for SpCas9 sgRNAs Targeting the MYOC
Coding sequence (larger batches). SpMCh10 sgRNA as an example.

| | Guide RNA | SEQ |
|---|---|---|
| Name | (Modified nucleotides in bold) | ID NO |
| SpMCh10 | asusgsCCAGUAUACCUUCAGUGGUUUUAGAgcuagaaauagcAAGUUAAAAUAAGGCUAGU | 371 |
| | CCGUUAUCaacuugaaaaaguggcaccgagucggugcusususu | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “about” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to ±10%, more preferably up to ±5%, and more preferably still up to f 1% of a given value. Where particular values are described in the application and claims, unless otherwise stated, the term “about” is implicit and in this context means within an acceptable error range for the particular value.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for delivering a CRISPR/Cas-mediated gene editing system to cells of the eye of a subject, the method comprising administering to the subject a plurality of lipid nanoparticles (LNPs) complexed with
(a) a guide RNA for a target gene or a nucleic acid encoding the guide RNA; and/or
(b) a RNA-guided endonuclease or a nucleic acid encoding the RNA-guided endonuclease, thereby reducing the expression of the target gene in cells of the eye of the subject.

2. The method of claim 1, wherein CRISPR/Cas-mediated gene editing system is delivered to the trabecular meshwork cells of the subject.

3. The method of claim 1, wherein the target gene is myocilin (MYOC) gene.

4. The method of claim 1, wherein the expression of the target gene, the expression of the protein encoded by the target gene, or both, in the subject's eye is reduced by at least 20% after the administration.

5. (canceled)

6. (canceled)

7. (canceled)

8. (canceled)

9. The method of claim 3, wherein the expression of the MYOC gene is reduced in the trabecular meshwork cells of the subject's eye by at least 20% after the administration.

10. The method of claim 1, wherein the RNA-guided nuclease is a Cas9 nuclease.

11. The method of claim 10, wherein the Cas9 nuclease is a Staphylococcus aureus Cas9 (SaCas9) nuclease or a Streptococcus pyogenes Cas9 (SpCas9) nuclease.

12. The method of claim 3, wherein the site targeted by the guide RNA is within exon 1, exon 2 or exon 3 of the MYOC gene.

13. The method of claim 3, wherein the site targeted by the guide RNA comprises a nucleotide sequence selected from the group consisting of SEQ ID NOs: 1-27 and 55-115.

14. (canceled)

15. (canceled)

16. The method of claim 3, wherein the guide RNA comprises a spacer sequence having a RNA sequence corresponding to any one of the nucleotide sequence selected from the group consisting of SEQ ID NOs: 1-27 and 55-115.

17. The method of claim 3, wherein the guide RNA comprises a spacer sequence having a RNA sequence corresponding to any one of the nucleotide sequence selected from the group consisting of SEQ ID NOs: 6, 10, 15, 18, 26, 59, 61, 63, 64, 66, 69, 72-77, 79, 81, 82, 90, 95, 98-101, 104, 106, 107, 109, and 113-115.

18. (canceled)

19. (canceled)

20. (canceled)

21. The method of claim 3, wherein the guide RNA comprises a nucleotide sequence selected from the group consisting of SEQ ID NOs: 258, 267-270, 309, 319, 328-331, 370 and 371.

22. The method of claim 1, wherein a LNP of the plurality of LNPs comprises an ionizable cationic lipid, a helper lipid, a sterol, and a poly(ethylene glycol)-lipid (PEG-lipid), and wherein the ionizable cationic lipid is selected from the group consisting of C12-200, cKK-E12,
DLIN-MC3, DLIN-MC4, DLIN-MC5, DODMA, DOTAP, DODAP, DC Cholesterol, DLin-DMA, DLin-K-DMA, and DLin-KC2 DMA;
the helper lipid is selected from the group consisting of 1,2-distearoyl-sn-glycero-3-phosphocholine (DSPC), 1,2-dioleoyl-sn-glycero-3-phosphocholine (DOPC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-phosphocholine (DOPC), diundecanoylphosphatidylcholine (DUPC), phosphatidylcholine (POPC), 1-stearoyl-2-oleoyl-sn-glycero-3-phosphocholine (SOPC), 1,2-dioleoyl-Sn-glycero-3-phosphoethanolamine (DOPE), 1,2-distearoyl-sn-glycero-3-phosphoethanolamine (DSPE), 1,2-dihexadecanoyl-sn-glycero-3-phosphoethanolamine, (DPPE), 1,2-dimyristoyl-sn-glycero-3-phosphoethanolamine (DMPE), 1-stearoyl-2-oleoyl-sn-glycero-3-phosphoethanolamine (SOPE, 18:0-18:1 PE), 1-palmitoyl-2-oleoyl-sn-glycero-3-phosphoethanolamine (POPE), dioleoyl phosphatidylglycerol (DOPG), and dipalmitoyl-sn-glycero-3-PG (DPPG):
the sterol is selected from the group consisting of cholesterol, sitosterol, p-sitosterol, phytosterols, fucosterol, zoosterol, and ergosterol; and/or
the PEG-lipid is DMG-PEG, DSG-PEG, a PEG-ceramide, or a PEG-phospholipid.

23. The method of claim 22, wherein the LNP comprises about 20-60% the ionizable lipids, about 18.5% to 60% the sterol, about 0.01 to 30% the helper lipid, and/or about 0%-10% PEG-lipid.

24-28. (canceled)

29. The method of claim 1, wherein a LNP of the plurality of LNPs comprises about 50 mol % of C12-200, DLIN-MC3, DODMA or DOTAP, about 10 mol % of DSPC, about 37.0-39.5 mol % of cholesterol or sitosterol, and about 0.5-3.0% of DMG-PEG.

30. (canceled)

31. (canceled)

32. The method of claim 1, wherein the plurality of LNP is administered to the subject by intravitreal injection or intracameral injection.

33. The method of claim 1, wherein the method comprises a single administration of the plurality of LNPs to the subject.

34. (canceled)

35. (canceled)

36. The method of claim 1, wherein the subject is a human.

37. The method of claim 1, wherein the LNPs are complexed with (a) the guide RNA or a nucleic acid encoding the guide RNA and (b) the RNA-guided endonuclease or the nucleic acid encoding the RNA-guided endonuclease separately, or wherein the LNPs complexed with (a) the guide RNA or a nucleic acid encoding the guide RNA and the LNPs complexed with (b) the RNA-guided endonuclease or the nucleic acid encoding the RNA-guided endonuclease are different LNPs.

38. (canceled)

39. A guide RNA targeting a MYOC gene, comprising a nucleotide sequence specific to a fragment in exon 1, exon 2 or exon 3 of the MYOC gene, wherein the guide RNA comprises a spacer sequence having a RNA sequence corresponding to any one of the nucleotide sequence selected from the group consisting of SEQ ID NOs: 1-27 and 55-115 or a spacer sequence having one, two, or three mismatches relative to a RNA sequence corresponding to any one of the nucleotide sequence selected from the group consisting of SEQ ID NOs: 1-27 and 55-115.

40-66. (canceled)


SEQUENCE LISTING (371 sequences)

SEQ ID NO: 1 - DNA, 21 bp, synthetic construct
  source 1..21: mol_type = other DNA
ctgcttctggcctgcctggtg

SEQ ID NO: 2 - DNA, 21 bp, synthetic construct
  source 1..21: mol_type = other DNA
cagctcaggaaggccaatgac

SEQ ID NO: 3 - DNA, 21 bp, synthetic construct
  source 1..21: mol_type = other DNA
ccttcagtgtggccagtccca

SEQ ID NO: 4 - DNA, 21 bp, synthetic construct
  source 1..21: mol_type = other DNA
gcctggctctgctctgggcag

SEQ ID NO: 5 - DNA, 21 bp, synthetic construct
  source 1..21: mol_type = other DNA
gctgctgtctctctgtaagtt

SEQ ID NO: 6 - DNA, 21 bp, synthetic construct
  source 1..21: mol_type = other DNA
ggtggcctccaggtctaagcg

SEQ ID NO: 7 - DNA, 21 bp, synthetic construct
  source 1..21: mol_type = other DNA
tggaggaggctctccagggag

SEQ ID NO: 8 - DNA, 21 bp, synthetic construct
  source 1..21: mol_type = other DNA
cagctccctctgcagcccctc

SEQ ID NO: 9 - DNA, 21 bp, synthetic construct
  source 1..21: mol_type = other DNA
cagctggtcccgctcccgcct

SEQ ID NO: 10 - DNA, 21 bp, synthetic construct
  source 1..21: mol_type = other DNA
accagctggaaacccaaacca

SEQ ID NO: 11 - DNA, 21 bp, synthetic construct
  source 1..21: mol_type = other DNA
ggcagtctccaactctctggt

SEQ ID NO: 12 - DNA, 21 bp, synthetic construct
  source 1..21: mol_type = other DNA
aagcgactaaggcaagaaaat

SEQ ID NO: 13 - DNA, 21 bp, synthetic construct
  source 1..21: mol_type = other DNA
tggcacagcccgagcagtgtc

SEQ ID NO: 14 - DNA, 21 bp, synthetic construct
  source 1..21: mol_type = other DNA
gctaactgaagttcctgcttc

SEQ ID NO: 15 - DNA, 21 bp, synthetic construct
  source 1..21: mol_type = other DNA
gagagcccatctggctatctc

SEQ ID NO: 16 - DNA, 21 bp, synthetic construct
  source 1..21: mol_type = other DNA
aattactggcaagtatggtgt

SEQ ID NO: 17 - DNA, 21 bp, synthetic construct
  source 1..21: mol_type = other DNA
gggtgtaggggtaggtgggct

SEQ ID NO: 18 - DNA, 21 bp, synthetic construct
  source 1..21: mol_type = other DNA
ctacacccaggagaccacgtg

SEQ ID NO: 19 - DNA, 21 bp, synthetic construct
  source 1..21: mol_type = other DNA
tccacgtggtctcctgggtgt

SEQ ID NO: 20 - DNA, 21 bp, synthetic construct
  source 1..21: mol_type = other DNA
gtcgattctccacgtggtctc

SEQ ID NO: 21 - DNA, 21 bp, synthetic construct
  source 1..21: mol_type = other DNA
tggagaatcgacacagttggc

SEQ ID NO: 22 - DNA, 21 bp, synthetic construct
  source 1..21: mol_type = other DNA
gcacggatgtccgccaggttt

SEQ ID NO: 23 - DNA, 21 bp, synthetic construct
  source 1..21: mol_type = other DNA
taggcagtatgtgaaccttag

SEQ ID NO: 24 - DNA, 21 bp, synthetic construct
  source 1..21: mol_type = other DNA
ctgcctaggccactggaaagc

SEQ ID NO: 25 - DNA, 21 bp, synthetic construct
  source 1..21: mol_type = other DNA
ggagcctctatttccagggcg

SEQ ID NO: 26 - DNA, 21 bp, synthetic construct
  source 1..21: mol_type = other DNA
cgccctggaaatagaggctcc

SEQ ID NO: 27 - DNA, 21 bp, synthetic construct
  source 1..21: mol_type = other DNA
agaactgtcataagatatgag

SEQ ID NO: 28 - DNA, 27 bp, synthetic construct
  source 1..27: mol_type = other DNA
ctgcttctggcctgcctggtgtgggat

SEQ ID NO: 29 - DNA, 27 bp, synthetic construct
  source 1..27: mol_type = other DNA
cagctcaggaaggccaatgaccagagt

SEQ ID NO: 30 - DNA, 27 bp, synthetic construct
  source 1..27: mol_type = other DNA
ccttcagtgtggccagtcccaatgaat

SEQ ID NO: 31 - DNA, 27 bp, synthetic construct
  source 1..27: mol_type = other DNA
gcctggctctgctctgggcagctggat

SEQ ID NO: 32 - DNA, 27 bp, synthetic construct
  source 1..27: mol_type = other DNA
gctgctgtctctctgtaagttatggat

SEQ ID NO: 33 - DNA, 27 bp, synthetic construct
  source 1..27: mol_type = other DNA
ggtggcctccaggtctaagcgttgggt

SEQ ID NO: 34 - DNA, 27 bp, synthetic construct
  source 1..27: mol_type = other DNA
tggaggaggctctccagggagctgagt

SEQ ID NO: 35 - DNA, 27 bp, synthetic construct
  source 1..27: mol_type = other DNA
cagctccctctgcagcccctcctgggt

SEQ ID NO: 36 - DNA, 27 bp, synthetic construct
  source 1..27: mol_type = other DNA
cagctggtcccgctcccgcctcagggt

SEQ ID NO: 37 - DNA, 27 bp, synthetic construct
  source 1..27: mol_type = other DNA
accagctggaaacccaaaccagagagt

SEQ ID NO: 38 - DNA, 27 bp, synthetic construct
  source 1..27: mol_type = other DNA
ggcagtctccaactctctggtttgggt

SEQ ID NO: 39 - DNA, 27 bp, synthetic construct
  source 1..27: mol_type = other DNA
aagcgactaaggcaagaaaatgagaat

SEQ ID NO: 40 - DNA, 27 bp, synthetic construct
  source 1..27: mol_type = other DNA
tggcacagcccgagcagtgtctcgggt

SEQ ID NO: 41 - DNA, 27 bp, synthetic construct
  source 1..27: mol_type = other DNA
gctaactgaagttcctgcttcccgaat

SEQ ID NO: 42 - DNA, 27 bp, synthetic construct
  source 1..27: mol_type = other DNA
gagagcccatctggctatctcaggagt

SEQ ID NO: 43 - DNA, 27 bp, synthetic construct
  source 1..27: mol_type = other DNA
aattactggcaagtatggtgtgtggat

SEQ ID NO: 44 - DNA, 27 bp, synthetic construct
  source 1..27: mol_type = other DNA
gggtgtaggggtaggtgggcttggggt

SEQ ID NO: 45 - DNA, 27 bp, synthetic construct
  source 1..27: mol_type = other DNA
ctacacccaggagaccacgtggagaat

SEQ ID NO: 46 - DNA, 27 bp, synthetic construct
  source 1..27: mol_type = other DNA
tccacgtggtctcctgggtgtaggggt

SEQ ID NO: 47 - DNA, 27 bp, synthetic construct
  source 1..27: mol_type = other DNA
gtcgattctccacgtggtctcctgggt

SEQ ID NO: 48 - DNA, 27 bp, synthetic construct
  source 1..27: mol_type = other DNA
tggagaatcgacacagttggcacggat

SEQ ID NO: 49 - DNA, 27 bp, synthetic construct
  source 1..27: mol_type = other DNA
gcacggatgtccgccaggtttttgagt

SEQ ID NO: 50 - DNA, 27 bp, synthetic construct
  source 1..27: mol_type = other DNA
taggcagtatgtgaaccttagaagggt

SEQ ID NO: 51 - DNA, 27 bp, synthetic construct
  source 1..27: mol_type = other DNA
ctgcctaggccactggaaagcacgggt

SEQ ID NO: 52 - DNA, 27 bp, synthetic construct
  source 1..27: mol_type = other DNA
ggagcctctatttccagggcgctgagt

SEQ ID NO: 53 - DNA, 27 bp, synthetic construct
  source 1..27: mol_type = other DNA
cgccctggaaatagaggctccccgagt

SEQ ID NO: 54 - DNA, 27 bp, synthetic construct
  source 1..27: mol_type = other DNA
agaactgtcataagatatgagctgaat

SEQ ID NO: 55 - DNA, 20 bp, synthetic construct
  source 1..20: mol_type = other DNA
gtgcacgttgctgcagcttt

SEQ ID NO: 56 - DNA, 20 bp, synthetic construct
  source 1..20: mol_type = other DNA
agctggacagctggcatctc

SEQ ID NO: 57 - DNA, 20 bp, synthetic construct
  source 1..20: mol_type = other DNA
agctgtccagctgctgcttc

SEQ ID NO: 58 - DNA, 20 bp, synthetic construct
  source 1..20: mol_type = other DNA
gcttctggcctgcctggtgt

SEQ ID NO: 59 - DNA, 20 bp, synthetic construct
  source 1..20: mol_type = other DNA
gcctgcctggtgtgggatgt

SEQ ID NO: 60 - DNA, 20 bp, synthetic construct
  source 1..20: mol_type = other DNA
ctgcctggtgtgggatgtgg

SEQ ID NO: 61 - DNA, 20 bp, synthetic construct
  source 1..20: mol_type = other DNA
gggccaggacagctcagctc

SEQ ID NO: 62 - DNA, 20 bp, synthetic construct
  source 1..20: mol_type = other DNA
gcatcggccactctggtcat

SEQ ID NO: 63 - DNA, 20 bp, synthetic construct
  source 1..20: mol_type = other DNA
tatactggcatcggccactc

SEQ ID NO: 64 - DNA, 20 bp, synthetic construct
  source 1..20: mol_type = other DNA
atgccagtataccttcagtg

SEQ ID NO: 65 - DNA, 20 bp, synthetic construct
  source 1..20: mol_type = other DNA
cactgaaggtatactggcat

SEQ ID NO: 66 - DNA, 20 bp, synthetic construct
  source 1..20: mol_type = other DNA
tggccacactgaaggtatac

SEQ ID NO: 67 - DNA, 20 bp, synthetic construct
  source 1..20: mol_type = other DNA
attgggactggccacactga

SEQ ID NO: 68 - DNA, 20 bp, synthetic construct
  source 1..20: mol_type = other DNA
gcagctggattcattgggac

SEQ ID NO: 69 - DNA, 20 bp, synthetic construct
  source 1..20: mol_type = other DNA
tctgggcagctggattcatt

SEQ ID NO: 70 - DNA, 20 bp, synthetic construct
  source 1..20: mol_type = other DNA
ctctgggcagctggattcat

SEQ ID NO: 71 - DNA, 20 bp, synthetic construct
  source 1..20: mol_type = other DNA
ctggctctgctctgggcagc

SEQ ID NO: 72 - DNA, 20 bp, synthetic construct
  source 1..20: mol_type = other DNA
taagttatggatgactgaca

SEQ ID NO: 73 - DNA, 20 bp, synthetic construct
  source 1..20: mol_type = other DNA
cagcacccaacgcttagacc

SEQ ID NO: 74 - DNA, 20 bp, synthetic construct
  source 1..20: mol_type = other DNA
cacccaacgcttagacctgg

SEQ ID NO: 75 - DNA, 20 bp, synthetic construct
  source 1..20: mol_type = other DNA
ggcctccaggtctaagcgtt

SEQ ID NO: 76 - DNA, 20 bp, synthetic construct
  source 1..20: mol_type = other DNA
tggcctccaggtctaagcgt

SEQ ID NO: 77 - DNA, 20 bp, synthetic construct
  source 1..20: mol_type = other DNA
gtcgagctttggtggcctcc

SEQ ID NO: 78 - DNA, 20 bp, synthetic construct
  source 1..20: mol_type = other DNA
ggagctgagtcgagctttgg

SEQ ID NO: 79 - DNA, 20 bp, synthetic construct
  source 1..20: mol_type = other DNA
caaagctcgactcagctccc

SEQ ID NO: 80 - DNA, 20 bp, synthetic construct
  source 1..20: mol_type = other DNA
cagggagctgagtcgagctt

SEQ ID NO: 81 - DNA, 20 bp, synthetic construct
  source 1..20: mol_type = other DNA
ttggtggaggaggctctcca

SEQ ID NO: 82 - DNA, 20 bp, synthetic construct
  source 1..20: mol_type = other DNA
attggtggaggaggctctcc

SEQ ID NO: 83 - DNA, 20 bp, synthetic construct
  source 1..20: mol_type = other DNA
cctcctccaccaattgacct

SEQ ID NO: 84 - DNA, 20 bp, synthetic construct
  source 1..20: mol_type = other DNA
ccaaggtcaattggtggagg

SEQ ID NO: 85 - DNA, 20 bp, synthetic construct
  source 1..20: mol_type = other DNA
ccaccaattgaccttggacc

SEQ ID NO: 86 - DNA, 20 bp, synthetic construct
  source 1..20: mol_type = other DNA
ggtccaaggtcaattggtgg

SEQ ID NO: 87 - DNA, 20 bp, synthetic construct
  source 1..20: mol_type = other DNA
cctggtccaaggtcaattgg

SEQ ID NO: 88 - DNA, 20 bp, synthetic construct
  source 1..20: mol_type = other DNA
cagcctggtccaaggtcaat

SEQ ID NO: 89 - DNA, 20 bp, synthetic construct
  source 1..20: mol_type = other DNA
gggcaccctgaggcgggagc

SEQ ID NO: 90 - DNA, 20 bp, synthetic construct
  source 1..20: mol_type = other DNA
ctggtcccgctcccgcctca

SEQ ID NO: 91 - DNA, 20 bp, synthetic construct
  source 1..20: mol_type = other DNA
gctggtcccgctcccgcctc

SEQ ID NO: 92 - DNA, 20 bp, synthetic construct
  source 1..20: mol_type = other DNA
gaggcgggagcgggaccagc

SEQ ID NO: 93 - DNA, 20 bp, synthetic construct
  source 1..20: mol_type = other DNA
ctctggtttgggtttccagc

SEQ ID NO: 94 - DNA, 20 bp, synthetic construct
  source 1..20: mol_type = other DNA
agtctccaactctctggttt

SEQ ID NO: 95 - DNA, 20 bp, synthetic construct
  source 1..20: mol_type = other DNA
gtctcggaggaggttgctgt

SEQ ID NO: 96 - DNA, 20 bp, synthetic construct
  source 1..20: mol_type = other DNA
cctccgagacaagtcagttc

SEQ ID NO: 97 - DNA, 20 bp, synthetic construct
  source 1..20: mol_type = other DNA
gaactgacttgtctcggagg

SEQ ID NO: 98 - DNA, 20 bp, synthetic construct
  source 1..20: mol_type = other DNA
ccagaactgacttgtctcgg

SEQ ID NO: 99 - DNA, 20 bp, synthetic construct
  source 1..20: mol_type = other DNA
ccgagacaagtcagttctgg

SEQ ID NO: 100 - DNA, 20 bp, synthetic construct
  source 1..20: mol_type = other DNA
cctccagaactgacttgtct

SEQ ID NO: 101 - DNA, 20 bp, synthetic construct
  source 1..20: mol_type = other DNA
aggaagagaagaagcgacta

SEQ ID NO: 102 - DNA, 20 bp, synthetic construct
  source 1..20: mol_type = other DNA
aagaaaatgagaatctggcc

SEQ ID NO: 103 - DNA, 20 bp, synthetic construct
  source 1..20: mol_type = other DNA
tgagaatctggccaggaggt

SEQ ID NO: 104 - DNA, 20 bp, synthetic construct
  source 1..20: mol_type = other DNA
gctgctgctttccaacctcc

SEQ ID NO: 105 - DNA, 20 bp, synthetic construct
  source 1..20: mol_type = other DNA
gaggttggaaagcagcagcc

SEQ ID NO: 106 - DNA, 20 bp, synthetic construct
  source 1..20: mol_type = other DNA
gttggaaagcagcagccagg

SEQ ID NO: 107 - DNA, 20 bp, synthetic construct
  source 1..20: mol_type = other DNA
gcagcagccaggaggtagca

SEQ ID NO: 108 - DNA, 20 bp, synthetic construct
  source 1..20: mol_type = other DNA
tgtctcgggtctggggacac

SEQ ID NO: 109 - DNA, 20 bp, synthetic construct
  source 1..20: mol_type = other DNA
ccagacccgagacactgctc

SEQ ID NO: 110 - DNA, 20 bp, synthetic construct
  source 1..20: mol_type = other DNA
cgagcagtgtctcgggtctg

SEQ ID NO: 111 - DNA, 20 bp, synthetic construct
  source 1..20: mol_type = other DNA
ccgagcagtgtctcgggtct

SEQ ID NO: 112 - DNA, 20 bp, synthetic construct
  source 1..20: mol_type = other DNA
cccgagcagtgtctcgggtc

SEQ ID NO: 113 - DNA, 20 bp, synthetic construct
  source 1..20: mol_type = other DNA
cacagcccgagcagtgtctc

SEQ ID NO: 114 - DNA, 20 bp, synthetic construct
  source 1..20: mol_type = other DNA
gcacagcccgagcagtgtct

SEQ ID NO: 115 - DNA, 20 bp, synthetic construct
  source 1..20: mol_type = other DNA
actgctcgggctgtgccacc

SEQ ID NO: 116 - DNA, 23 bp, synthetic construct
  source 1..23: mol_type = other DNA
gtgcacgttgctgcagctttggg

SEQ ID NO: 117 - DNA, 23 bp, synthetic construct
  source 1..23: mol_type = other DNA
agctggacagctggcatctcagg

SEQ ID NO: 118 - DNA, 23 bp, synthetic construct
  source 1..23: mol_type = other DNA
agctgtccagctgctgcttctgg

SEQ ID NO: 119 - DNA, 23 bp, synthetic construct
  source 1..23: mol_type = other DNA
gcttctggcctgcctggtgtggg

SEQ ID NO: 120 - DNA, 23 bp, synthetic construct
  source 1..23: mol_type = other DNA
gcctgcctggtgtgggatgtggg

SEQ ID NO: 121 - DNA, 23 bp, synthetic construct
  source 1..23: mol_type = other DNA
ctgcctggtgtgggatgtggggg

SEQ ID NO: 122 - DNA, 23 bp, synthetic construct
  source 1..23: mol_type = other DNA
gggccaggacagctcagctcagg

SEQ ID NO: 123 - DNA, 23 bp, synthetic construct
  source 1..23: mol_type = other DNA
gcatcggccactctggtcattgg

SEQ ID NO: 124 - DNA, 23 bp, synthetic construct
  source 1..23: mol_type = other DNA
tatactggcatcggccactctgg

SEQ ID NO: 125 - DNA, 23 bp, synthetic construct
  source 1..23: mol_type = other DNA
atgccagtataccttcagtgtgg

SEQ ID NO: 126 - DNA, 23 bp, synthetic construct
  source 1..23: mol_type = other DNA
cactgaaggtatactggcatcgg

SEQ ID NO: 127 - DNA, 23 bp, synthetic construct
  source 1..23: mol_type = other DNA
tggccacactgaaggtatactgg

SEQ ID NO: 128 - DNA, 23 bp, synthetic construct
  source 1..23: mol_type = other DNA
attgggactggccacactgaagg

SEQ ID NO: 129 - DNA, 23 bp, synthetic construct
  source 1..23: mol_type = other DNA
gcagctggattcattgggactgg

SEQ ID NO: 130 - DNA, 23 bp, synthetic construct
  source 1..23: mol_type = other DNA
tctgggcagctggattcattggg

SEQ ID NO: 131 - DNA, 23 bp, synthetic construct
  source 1..23: mol_type = other DNA
ctctgggcagctggattcattgg

SEQ ID NO: 132 - DNA, 23 bp, synthetic construct
  source 1..23: mol_type = other DNA
ctggctctgctctgggcagctgg

SEQ ID NO: 133 - DNA, 23 bp, synthetic construct
  source 1..23: mol_type = other DNA
taagttatggatgactgacatgg

SEQ ID NO: 134 - DNA, 23 bp, synthetic construct
  source 1..23: mol_type = other DNA
cagcacccaacgcttagacctgg

SEQ ID NO: 135 - DNA, 23 bp, synthetic construct
  source 1..23: mol_type = other DNA
cacccaacgcttagacctggagg

SEQ ID NO: 136 - DNA, 23 bp, synthetic construct
  source 1..23: mol_type = other DNA
ggcctccaggtctaagcgttggg

SEQ ID NO: 137 - DNA, 23 bp, synthetic construct
  source 1..23: mol_type = other DNA
tggcctccaggtctaagcgttgg

SEQ ID NO: 138 - DNA, 23 bp, synthetic construct
  source 1..23: mol_type = other DNA
gtcgagctttggtggcctccagg

SEQ ID NO: 139 - DNA, 23 bp, synthetic construct
  source 1..23: mol_type = other DNA
ggagctgagtcgagctttggtgg

SEQ ID NO: 140 - DNA, 23 bp, synthetic construct
  source 1..23: mol_type = other DNA
caaagctcgactcagctccctgg

SEQ ID NO: 141 - DNA, 23 bp, synthetic construct
  source 1..23: mol_type = other DNA
cagggagctgagtcgagctttgg

SEQ ID NO: 142 - DNA, 23 bp, synthetic construct
  source 1..23: mol_type = other DNA
ttggtggaggaggctctccaggg

SEQ ID NO: 143 - DNA, 23 bp, synthetic construct
  source 1..23: mol_type = other DNA
attggtggaggaggctctccagg

SEQ ID NO: 144 - DNA, 23 bp, synthetic construct
  source 1..23: mol_type = other DNA
cctcctccaccaattgaccttgg

SEQ ID NO: 145 - DNA, 23 bp, synthetic construct
  source 1..23: mol_type = other DNA
ccaaggtcaattggtggaggagg

SEQ ID NO: 146 - DNA, 23 bp, synthetic construct
  source 1..23: mol_type = other DNA
ccaccaattgaccttggaccagg

SEQ ID NO: 147 - DNA, 23 bp, synthetic construct
  source 1..23: mol_type = other DNA
ggtccaaggtcaattggtggagg

SEQ ID NO: 148 - DNA, 23 bp, synthetic construct
  source 1..23: mol_type = other DNA
cctggtccaaggtcaattggtgg

SEQ ID NO: 149 - DNA, 23 bp, synthetic construct
  source 1..23: mol_type = other DNA
cagcctggtccaaggtcaattgg

SEQ ID NO: 150 - DNA, 23 bp, synthetic construct
  source 1..23: mol_type = other DNA
gggcaccctgaggcgggagcggg

SEQ ID NO: 151 - DNA, 23 bp, synthetic construct
  source 1..23: mol_type = other DNA
ctggtcccgctcccgcctcaggg

SEQ ID NO: 152 - DNA, 23 bp, synthetic construct
  source 1..23: mol_type = other DNA
gctggtcccgctcccgcctcagg

SEQ ID NO: 153 - DNA, 23 bp, synthetic construct
  source 1..23: mol_type = other DNA
gaggcgggagcgggaccagctgg

SEQ ID NO: 154 - DNA, 23 bp, synthetic construct
  source 1..23: mol_type = other DNA
ctctggtttgggtttccagctgg

SEQ ID NO: 155 - DNA, 23 bp, synthetic construct
  source 1..23: mol_type = other DNA
agtctccaactctctggtttggg

SEQ ID NO: 156 - DNA, 23 bp, synthetic construct
  source 1..23: mol_type = other DNA
gtctcggaggaggttgctgtagg

SEQ ID NO: 157 - DNA, 23 bp, synthetic construct
  source 1..23: mol_type = other DNA
cctccgagacaagtcagttctgg

SEQ ID NO: 158 - DNA, 23 bp, synthetic construct
  source 1..23: mol_type = other DNA
gaactgacttgtctcggaggagg

SEQ ID NO: 159 - DNA, 23 bp, synthetic construct
  source 1..23: mol_type = other DNA
ccagaactgacttgtctcggagg

SEQ ID NO: 160 - DNA, 23 bp, synthetic construct
  source 1..23: mol_type = other DNA
ccgagacaagtcagttctggagg

SEQ ID NO: 161 - DNA, 23 bp, synthetic construct
  source 1..23: mol_type = other DNA
cctccagaactgacttgtctcgg

SEQ ID NO: 162 - DNA, 23 bp, synthetic construct
  source 1..23: mol_type = other DNA
aggaagagaagaagcgactaagg

SEQ ID NO: 163 - DNA, 23 bp, synthetic construct
  source 1..23: mol_type = other DNA
aagaaaatgagaatctggccagg

SEQ ID NO: 164 - DNA, 23 bp, synthetic construct
  source 1..23: mol_type = other DNA
tgagaatctggccaggaggttgg

SEQ ID NO: 165 - DNA, 23 bp, synthetic construct
  source 1..23: mol_type = other DNA
gctgctgctttccaacctcctgg

SEQ ID NO: 166 - DNA, 23 bp, synthetic construct
  source 1..23: mol_type = other DNA
gaggttggaaagcagcagccagg

SEQ ID NO: 167 - DNA, 23 bp, synthetic construct
  source 1..23: mol_type = other DNA
gttggaaagcagcagccaggagg

SEQ ID NO: 168 - DNA, 23 bp, synthetic construct
  source 1..23: mol_type = other DNA
gcagcagccaggaggtagcaagg

SEQ ID NO: 169 - DNA, 23 bp, synthetic construct
  source 1..23: mol_type = other DNA
tgtctcgggtctggggacactgg

SEQ ID NO: 170 - DNA, 23 bp, synthetic construct
  source 1..23: mol_type = other DNA
ccagacccgagacactgctcggg

SEQ ID NO: 171 - DNA, 23 bp, synthetic construct
  source 1..23: mol_type = other DNA
cgagcagtgtctcgggtctgggg

SEQ ID NO: 172 - DNA, 23 bp, synthetic construct
  source 1..23: mol_type = other DNA
ccgagcagtgtctcgggtctggg

SEQ ID NO: 173 - DNA, 23 bp, synthetic construct
  source 1..23: mol_type = other DNA
cccgagcagtgtctcgggtctgg

SEQ ID NO: 174 - DNA, 23 bp, synthetic construct
  source 1..23: mol_type = other DNA
cacagcccgagcagtgtctcggg

SEQ ID NO: 175 - DNA, 23 bp, synthetic construct
  source 1..23: mol_type = other DNA
gcacagcccgagcagtgtctcgg

SEQ ID NO: 176 - DNA, 23 bp, synthetic construct
  source 1..23: mol_type = other DNA
actgctcgggctgtgccaccagg

SEQ ID NO: 177 - RNA, 77 nt, synthetic construct
  source 1..77: mol_type = other RNA
gttttagagctagaaatagcaagttaaaataaggctagtccgttatcaacttgaaaaagtggcaccgagtcggtgct

SEQ ID NO: 178 - RNA, 86 nt, synthetic construct
  source 1..86: mol_type = other RNA
gtttaagtactctgtgctggaaacagcacagaatctacttaaacaaggcaaaatgccgtgtttatctcgtcaacttgttggcgaga

SEQ ID NO: 179 - DNA, 22 bp, synthetic construct
  source 1..22: mol_type = other DNA
gggtgcataaattgggatgttc

SEQ ID NO: 180 - DNA, 22 bp, synthetic construct
  source 1..22: mol_type = other DNA
tacttgaaggtgatcgctgtgc

SEQ ID NO: 181 - DNA, 20 bp, synthetic construct
  source 1..20: mol_type = other DNA
ctgtcttcccccatgaaggg

SEQ ID NO: 182 - DNA, 22 bp, synthetic construct
  source 1..22: mol_type = other DNA
ctctctggtttgggtttccagc

SEQ ID NO: 183 - DNA, 20 bp, synthetic construct
  source 1..20: mol_type = other DNA
gacagctcagctcaggaagg

SEQ ID NO: 184 - DNA, 20 bp, synthetic construct
  source 1..20: mol_type = other DNA
gctgctgctttccaacctcc

SEQ ID NO: 185 - DNA, 22 bp, synthetic construct
  source 1..22: mol_type = other DNA
tacttgaaggtgatcgctgtgc

SEQ ID NO: 186 - DNA, 20 bp, synthetic construct
  source 1..20: mol_type = other DNA
ccaaagctcgactcagctcc

SEQ ID NO: 187 - DNA, 23 bp, synthetic construct
  source 1..23: mol_type = other DNA
ctgccaccacatccagctaattc

SEQ ID NO: 188 - DNA, 24 bp, synthetic construct
  source 1..24: mol_type = other DNA
tgggtgataggatagagggctttg

SEQ ID NO: 189 - DNA, 21 bp, synthetic construct
  source 1..21: mol_type = other DNA
cgatttgtctccagggctgtc

SEQ ID NO: 190 - DNA, 22 bp, synthetic construct
  source 1..22: mol_type = other DNA
gtttggagaggacaatggcacc

SEQ ID NO: 191 - DNA, 22 bp, synthetic construct
  source 1..22: mol_type = other DNA
tctccagggctgtcacatctac

SEQ ID NO: 192 - DNA, 20 bp, synthetic construct
  source 1..20: mol_type = other DNA
ctttggcctcatcggtgctg

SEQ ID NO: 193 - DNA, 4176 bp, Streptococcus pyogenes
  source 1..4176: mol_type = genomic DNA
atgggccccgccgccaagagagtgaagctggacggatccgacaagaagtactccattgggctggacattggcactaactccgtgggatgggccgtgatcaccgacgagtacaaagtgcccagcaagaagtttaaagtgctggggaatactgaccggcacagcatcaagaagaaccttataggcgccctcctgtttgattccggagaaaccgctgaagccacccggctcaagagaaccgccagacgccgctacacccggaggaagaatcgcatctgctatctgcaagagatcttctccaacgaaatggccaaggtggacgactcgttcttccatcggctggaggagtcctttctggtggaagaagataagaagcatgagagacaccccatcttcggcaacatcgtggatgaagtggcctaccacgaaaagtaccctaccatctaccaccttcgcaagaagctcgtggatagcactgataaggcggacctccgcctgatctacctcgcgctcgcccatatgatcaagttccggggacacttcctgatcgagggggacctgaaccctgacaacagcgacgtggataagctgttcatccaactggtgcaaacctataaccagctgttcgaggagaaccctatcaacgcctccggagtggacgccaaggccatcctgtcggctcgcctgtccaagtcgagaaggctggaaaacctgattgcccagctcccgggagaaaagaagaacggcctgttcggcaacctgatcgctctctccctgggcctgaccccgaatttcaagagcaacttcgacctcgccgaagatgcaaagctccagctgtcaaaagacacctacgacgatgacctggacaatctgctggcacagatcggggatcagtacgctgacctgttcctggccgccaagaacctgtccgacgcgatcctgctctcggatattctgagggtcaacaccgagattaccaaggcccctctgtccgcgagcatgatcaagcggtacgatgaacatcaccaggatctgacactcttgaaggcccttgtccgccaacaactgccggagaagtacaaggagattttctttgatcagtccaagaacggctacgctggctacattgacgggggtgccagccaggaagaattttacaagttcattaagcctattctcgaaaagatggacggaactgaggagttgctcgtgaagctgaaccgggaggacctgttgagaaagcaacgcaccttcgacaacggttcgattcctcatcaaattcatctgggtgaactgcacgccatcctccggcggcaggaggatttctatccattcctgaaagacaaccgagagaagattgagaaaatcctgaccttccggataccctactacgtgggaccattggctcgggggaacagcagattcgcgtggatgactagaaagtccgaggagactattaccccgtggaacttcgaggaggtggtcgataagggcgcatcggcacagtccttcatcgagcggatgaccaacttcgacaagaaccttcccaacgaaaaggtgctgcccaagcactcgctgttgtacgagtactttaccgtgtacaacgagctcactaaagtgaaatacgtgaccgagggaatgagaaagccggcctttctgtccggggaacagaagaaggccatcgtggacctcctcttcaaaaccaacagaaaagtcaccgtgaagcagctgaaggaggactacttcaagaaaatcgagtgcttcgactcggtcgagatttcgggggtcgaggataggtttaatgccagcctgggtacttaccacgatctgctgaagatcattaaggacaaggacttccttgacaacgaagaaaacgaggacatccttgaggacattgtcctgaccctgaccctgtttgaggatcgggagatgattgaggaaagacttaagacctacgctcatttgttcgacgacaaggtcatgaaacagctgaagcggaggcggtacactggatggggtcggctgtccaggaagctgatcaacggaatccgggacaagcaatccggaaagaccatcctggacttcctgaagtcagacgggttcgccaaccggaacttcatgcagctcattcacgacgacagcctgacgttcaaggaggacatccagaaggcacaagtgtcgggacagggagacagcctccacgaacacattgcgaacctcgcgggttcaccggctatcaagaagggaatcctgcagactgtgaaggtggtggacgagttggtcaaggtcatgggcaggcataagcctgaaaacatcgtgatcgagatggcccgggagaaccagaccacccagaaggggcagaagaacagcagagagcgcatgaagcgcattgaggagggcatcaaggaactgggatcacagatcctgaaggaacatcccgtggaaaacacgcagctgcagaacgagaaactctacctgtactatttgcaaaacggccgcgatatgtacgtggaccaagaactcgatatcaaccgcctgtccgactacgacgtggaccacatcgtgccgcagagcttcctgaaggatgattctatcgataacaaggtcctcacccggtcggacaagaatcgggggaagtcagataacgtgccgtctgaggaagtggtgaagaagatgaagaattactggcggcagcttctgaacgcgaaacttattacccagcggaaattcgacaacctgactaaggccgagcggggaggactgtcagaactggacaaagccggcttcattaagagacagctggtcgaaactcgccagatcaccaaacatgtggcccagatcctggactccaggatgaacaccaagtacgacgaaaacgataagctcattcgggaagtgaaagtgatcacactgaagtccaagctggtgtccgacttccgcaaggacttccagttctacaaggtccgcgagattaacaactaccaccacgcacacgacgcttacttgaacgccgtcgtgggcactgccttgattaagaaatacccgaagctggaatccgagttcgtgtacggagactacaaggtgtacgatgtgcgcaagatgatcgccaagtcggagcaagaaatcggaaaggccaccgctaagtatttcttttactccaacattatgaacttcttcaagactgagatcaccctggccaatggagaaatccgcaagaggccgctgatcgaaaccaatggagagactggagagattgtgtgggataagggacgcgacttcgccaccgtgcgcaaggtgctgagcatgccccaagtcaacattgtgaaaaagaccgaagtgcagacgggcggtttctcaaaggaaagcatcctgcctaagcggaactccgataagctgatcgcgcgcaagaaggactgggacccgaagaaatatggcggcttcgactcccccaccgtcgcctactcggtgctcgtcgtggctaaagtggagaagggaaagtcgaagaagctcaagtccgtgaaggaattgctgggtattactattatggaacggtccagcttcgagaagaatccgatcgacttcctggaggccaagggatacaaggaagtgaagaaggacctgatcattaagctgccgaagtacagcctttttgagctggaaaacggacgcaagcggatgctggcctccgccggagagctgcagaagggcaacgaactggccctcccgtccaaatacgtgaactttctgtacctggccagccactacgagaagctgaagggatcacctgaagataacgagcagaagcagctgttcgtggaacaacataagcattatcttgacgagatcattgaacagatctctgagttctccaagagagtgattctggctgacgctaaccttgacaaagtgctgagcgcttacaacaagcacagggacaagcccatccgggagcaggcagagaacatcattcacctgttcactctcaccaacttgggtgccccggcagccttcaagtacttcgataccacaatcgaccgcaagaggtacacctcaaccaaggaggtccttgacgctaccctgatccatcaatccattaccggcctgtacgaaactaggatcgacctgtcgcagctgggtggcgacaagcttcctgccgccaagagagtgaagctggactaa

SEQ ID NO: 194 - DNA, 3231 bp, Staphylococcus aureus
  source 1..3231: mol_type = genomic DNA
atgcctgccgccaagagagtgaagctggacggatccggaaagcggaactatatcctgggactggacatcggaattacctccgtgggatacggcatcatcgattacgagactagggacgtgattgacgccggcgtgagactctttaaggaggccaacgtggaaaacaacgaaggtcgcagatccaagcggggtgcaagacgcctgaagcgccggaggagacatcggatacagcgcgtgaagaagctccttttcgactacaacctcctcactgaccactcggaattgtccggtatcaacccctacgaagcccgcgtgaaaggcctgagccagaagctgtccgaagaggagtttagcgcagccctgctgcacctggctaagcgaaggggggtgcacaacgtgaacgaggtggaggaggacactggcaacgaactgtccaccaaggagcagatttcacggaactcgaaggcgctggaagagaaatatgtggccgagctgcagctggagaggctcaagaaggatggcgaagtccgggggagcatcaatcgcttcaagacctcggactacgtgaaggaagccaaacagctgttgaaggtgcagaaggcctaccaccaactggaccaatcattcattgacacttacatcgatctgcttgaaaccaggcgcacctactacgagggtcctggagaaggcagccctttcggatggaaggacatcaaggagtggtatgagatgctgatgggtcattgcacctactttccggaagaactgcgctcagtgaagtacgcgtacaacgctgacctctacaacgctctcaacgatctgaacaacctcgtgatcacccgggacgagaacgaaaagctggagtactacgaaaagttccagattatcgaaaacgtgttcaagcagaagaagaagcccaccctgaagcagattgcaaaggagatccttgtgaacgaggaggatattaagggctaccgggtcacctccaccgggaaaccagagttcactaatctcaaggtgtaccatgacattaaggacattactgcccgcaaggagatcattgaaaacgcggaactgctggaccaaatcgcgaagatcctgaccatctatcagagctccgaggatatccaggaggaacttactaacctcaattccgagctgacgcaggaagaaatcgagcaaattagcaacctgaagggttacactggaacccacaacctcagcttgaaagcgattaaccttattttggatgaactttggcacactaatgacaatcagatcgccattttcaaccggctgaaactggtgccgaagaaggtggacctgagccaacagaaggaaatcccgaccacccttgtggacgatttcatcctgtcacctgtggtgaagaggagcttcatccagtcgatcaaggtcatcaacgccatcataaagaagtacggccttcccaacgacatcatcatcgaactggcccgcgagaagaactccaaagatgcccagaagatgatcaacgagatgcagaagcgaaaccggcagacgaacgaacggatcgaggagatcatccggaccaccgggaaggaaaacgcgaagtacctgatcgagaaaatcaagctgcatgatatgcaggaagggaagtgtctctactccctggaggccattccgctggaggatttgctgaacaaccctttcaactacgaagtcgatcatatcattcctcgctccgtgtccttcgataactccttcaacaataaggtcctcgtgaagcaggaggagaactcgaagaagggcaacagaaccccgttccagtacctctcgtcgtccgactccaagatcagctacgaaactttcaagaagcacattctgaacctggccaagggcaaagggagaattagcaagaccaagaaggaatacctcctggaagagagagacatcaaccgcttctcggtgcaaaaggatttcatcaaccgcaacctggtcgataccagatacgccaccaggggactgatgaacctcctgcggtcctacttccgggtcaacaatctggacgtgaaggtcaaatccatcaacgggggctttacttctttcctgcgccggaagtggaagttcaagaaggaacggaacaagggatacaagcaccacgctgaagatgccctgattattgccaacgccgacttcatctttaaggaatggaaaaagctggacaaggctaagaaggtcatggagaaccagatgttcgaagaaaagcaggccgagtccatgcccgaaatcgaaaccgagcaggaatacaaggagatcttcatcacaccgcaccaaatcaagcacatcaaggacttcaaggattacaagtacagccaccgggtggacaagaagcctaacagagagcttatcaacgacaccctgtactccacgcgcaaggacgacaagggaaacacattgatcgtgaacaacctgaacggactgtatgacaaggacaatgacaaactgaagaagctgatcaacaaatcgccggaaaagctcctgatgtaccatcacgaccctcaaacctaccagaaactgaagctcatcatggagcagtacggcgacgaaaagaatcccctgtacaaatactacgaggagactggaaattacctgactaagtactccaagaaggataacggccccgtgatcaagaagattaagtactacggaaacaaactgaacgcacatctcgacatcaccgatgattatccaaactcccgcaacaaagtcgtgaagctctccctcaaaccgtaccgcttcgacgtgtacctggataatggggtgtacaagttcgtgaccgtgaagaacctggacgtcattaagaaggaaaactactacgaagtgaactcaaagtgctacgaggaagccaagaagctcaagaagatcagcaaccaggccgagttcatcgcatcgttttacaacaatgacctcattaagattaatggagaactgtacagagtgatcggcgtgaacaacgacctcctgaaccggattgaagtgaacatgatcgatattacctaccgggagtatctggagaacatgaacgacaagcgcccaccgagaatcatcaaaactattgcctccaagacccaatccattaagaaatactccaccgacatcctgggcaacctgtacgaggtcaagtcgaagaagcacccccagattatcaagaagggaaagcttcctgccgccaagagagtgaagctggactaa

SEQ ID NO: 195 - RNA, 98 nt, synthetic construct
  source 1..98: mol_type = other RNA
ctgcttctggcctgcctggtggttttagtactctggaaacagaatctactaaaacaaggcaaaatgccgtgtttatctcgtcaacttgttggcgagat

SEQ ID NO: 196 - RNA, 98 nt, synthetic construct
  source 1..98: mol_type = other RNA
cagctcaggaaggccaatgacgttttagtactctggaaacagaatctactaaaacaaggcaaaatgccgtgtttatctcgtcaacttgttggcgagat

SEQ ID NO: 197 - DNA, 98 bp, synthetic construct
  source 1..98: mol_type = other DNA
ccttcagtgtggccagtcccagttttagtactctggaaacagaatctactaaaacaaggcaaaatgccgtgtttatctcgtcaacttgttggcgagat

SEQ ID NO: 198 - RNA, 98 nt, synthetic construct
  source 1..98: mol_type = other RNA
gcctggctctgctctgggcaggttttagtactctggaaacagaatctactaaaacaaggcaaaatgccgtgtttatctcgtcaacttgttggcgagat

SEQ ID NO: 199 - RNA, 98 nt, synthetic construct
  source 1..98: mol_type = other RNA
gctgctgtctctctgtaagttgttttagtactctggaaacagaatctactaaaacaaggcaaaatgccgtgtttatctcgtcaacttgttggcgagat

SEQ ID NO: 200 - RNA, 98 nt, synthetic construct
  source 1..98: mol_type = other RNA
ggtggcctccaggtctaagcggttttagtactctggaaacagaatctactaaaacaaggcaaaatgccgtgtttatctcgtcaacttgttggcgagat

SEQ ID NO: 201 - RNA, 98 nt, synthetic construct
  source 1..98: mol_type = other RNA
tggaggaggctctccagggaggttttagtactctggaaacagaatctactaaaacaaggcaaaatgccgtgtttatctcgtcaacttgttggcgagat

SEQ ID NO: 202 - RNA, 98 nt, synthetic construct
  source 1..98: mol_type = other RNA
cagctccctctgcagcccctcgttttagtactctggaaacagaatctactaaaacaaggcaaaatgccgtgtttatctcgtcaacttgttggcgagat

SEQ ID NO: 203 - RNA, 98 nt, synthetic construct
  source 1..98: mol_type = other RNA
cagctggtcccgctcccgcctgttttagtactctggaaacagaatctactaaaacaaggcaaaatgccgtgtttatctcgtcaacttgttggcgagat

SEQ ID NO: 204 - RNA, 98 nt, synthetic construct
  source 1..98: mol_type = other RNA
accagctggaaacccaaaccagttttagtactctggaaacagaatctactaaaacaaggcaaaatgccgtgtttatctcgtcaacttgttggcgagat

SEQ ID NO: 205 - RNA, 98 nt, synthetic construct
  source 1..98: mol_type = other RNA
ggcagtctccaactctctggtgttttagtactctggaaacagaatctactaaaacaaggcaaaatgccgtgtttatctcgtcaacttgttggcgagat

SEQ ID NO: 206 - RNA, 98 nt, synthetic construct
  source 1..98: mol_type = other RNA
aagcgactaaggcaagaaaatgttttagtactctggaaacagaatctactaaaacaaggcaaaatgccgtgtttatctcgtcaacttgttggcgagat

SEQ ID NO: 207 - RNA, 98 nt, synthetic construct
  source 1..98: mol_type = other RNA
tggcacagcccgagcagtgtcgttttagtactctggaaacagaatctactaaaacaaggcaaaatgccgtgtttatctcgtcaacttgttggcgagat

SEQ ID NO: 208 - RNA, 98 nt, synthetic construct
  source 1..98: mol_type = other RNA
gctaactgaagttcctgcttcgttttagtactctggaaacagaatctactaaaacaaggcaaaatgccgtgtttatctcgtcaacttgttggcgagat

SEQ ID NO: 209 - RNA, 98 nt, synthetic construct
  source 1..98: mol_type = other RNA
gagagcccatctggctatctcgttttagtactctggaaacagaatctactaaaacaaggcaaaatgccgtgtttatctcgtcaacttgttggcgagat

SEQ ID NO: 210 - RNA, 98 nt, synthetic construct
  source 1..98: mol_type = other RNA
aattactggcaagtatggtgtgttttagtactctggaaacagaatctactaaaacaaggcaaaatgccgtgtttatctcgtcaacttgttggcgagat

SEQ ID NO: 211 - RNA, 98 nt, synthetic construct
  source 1..98: mol_type = other RNA
gggtgtaggggtaggtgggctgttttagtactctggaaacagaatctactaaaacaaggcaaaatgccgtgtttatctcgtcaacttgttggcgagat

SEQ ID NO: 212 - RNA, 98 nt, synthetic construct
  source 1..98: mol_type = other RNA
ctacacccaggagaccacgtggttttagtactctggaaacagaatctactaaaacaaggcaaaatgccgtgtttatctcgtcaacttgttggcgagat

SEQ ID NO: 213 - RNA, 98 nt, synthetic construct
  source 1..98: mol_type = other RNA
tccacgtggtctcctgggtgtgttttagtactctggaaacagaatctactaaaacaaggcaaaatgccgtgtttatctcgtcaacttgttggcgagat

SEQ ID NO: 214 - RNA, 98 nt, synthetic construct
  source 1..98: mol_type = other RNA
gtcgattctccacgtggtctcgttttagtactctggaaacagaatctactaaaacaaggcaaaatgccgtgtttatctcgtcaacttgttggcgagat

SEQ ID NO: 215 - RNA, 98 nt, synthetic construct
  source 1..98: mol_type = other RNA
tggagaatcgacacagttggcgttttagtactctggaaacagaatctactaaaacaaggcaaaatgccgtgtttatctcgtcaacttgttggcgagat

SEQ ID NO: 216 - RNA, 98 nt, synthetic construct
  source 1..98: mol_type = other RNA
gcacggatgtccgccaggtttgttttagtactctggaaacagaatctactaaaacaaggcaaaatgccgtgtttatctcgtcaacttgttggcgagat

SEQ ID NO: 217 - RNA, 98 nt, synthetic construct
  source 1..98: mol_type = other RNA
taggcagtatgtgaaccttaggttttagtactctggaaacagaatctactaaaacaaggcaaaatgccgtgtttatctcgtcaacttgttggcgagat

SEQ ID NO: 218 - RNA, 98 nt, synthetic construct
  source 1..98: mol_type = other RNA
ctgcctaggccactggaaagcgttttagtactctggaaacagaatctactaaaacaaggcaaaatgccgtgtttatctcgtcaacttgttggcgagat

SEQ ID NO: 219 - RNA, 98 nt, synthetic construct
  source 1..98: mol_type = other RNA
ggagcctctatttccagggcggttttagtactctggaaacagaatctactaaaacaaggcaaaatgccgtgtttatctcgtcaacttgttggcgagat

SEQ ID NO: 220 - RNA, 98 nt, synthetic construct
  source 1..98: mol_type = other RNA
cgccctggaaatagaggctccgttttagtactctggaaacagaatctactaaaacaaggcaaaatgccgtgtttatctcgtcaacttgttggcgagat

SEQ ID NO: 221 - RNA, 98 nt, synthetic construct
  source 1..98: mol_type = other RNA
agaactgtcataagatatgaggttttagtactctggaaacagaatctactaaaacaaggcaaaatgccgtgtttatctcgtcaacttgttggcgagat

SEQ ID NO: 222 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = Phosphorothioate linkage
ctgcttctggcctgcctggtggttttagtactctggaaacagaatctactaaaacaaggcaaaatgccgtgtttatctcgtcaacttgttggcgagatttt

SEQ ID NO: 223 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
cagctcaggaaggccaatgacgttttagtactctggaaacagaatctactaaaacaaggcaaaatgccgtgtttatctcgtcaacttgttggcgagatttt

SEQ ID NO: 224 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
ccttcagtgtggccagtcccagttttagtactctggaaacagaatctactaaaacaaggcaaaatgccgtgtttatctcgtcaacttgttggcgagatttt

SEQ ID NO: 225 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
gcctggctctgctctgggcaggttttagtactctggaaacagaatctactaaaacaaggcaaaatgccgtgtttatctcgtcaacttgttggcgagatttt

SEQ ID NO: 226 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
gctgctgtctctctgtaagttgttttagtactctggaaacagaatctactaaaacaaggcaaaatgccgtgtttatctcgtcaacttgttggcgagatttt

SEQ ID NO: 227 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
ggtggcctccaggtctaagcggttttagtactctggaaacagaatctactaaaacaaggcaaaatgccgtgtttatctcgtcaacttgttggcgagatttt

SEQ ID NO: 228 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
tggaggaggctctccagggaggttttagtactctggaaacagaatctactaaaacaaggcaaaatgccgtgtttatctcgtcaacttgttggcgagatttt

SEQ ID NO: 229 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
cagctccctctgcagcccctcgttttagtactctggaaacagaatctactaaaacaaggcaaaatgccgtgtttatctcgtcaacttgttggcgagatttt

SEQ ID NO: 230 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
cagctggtcccgctcccgcctgttttagtactctggaaacagaatctactaaaacaaggcaaaatgccgtgtttatctcgtcaacttgttggcgagatttt

SEQ ID NO: 231 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
accagctggaaacccaaaccagttttagtactctggaaacagaatctactaaaacaaggcaaaatgccgtgtttatctcgtcaacttgttggcgagatttt

SEQ ID NO: 232 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
ggcagtctccaactctctggtgttttagtactctggaaacagaatctactaaaacaaggcaaaatgccgtgtttatctcgtcaacttgttggcgagatttt

SEQ ID NO: 233 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
aagcgactaaggcaagaaaatgttttagtactctggaaacagaatctactaaaacaaggcaaaatgccgtgtttatctcgtcaacttgttggcgagatttt

SEQ ID NO: 234 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
tggcacagcccgagcagtgtcgttttagtactctggaaacagaatctactaaaacaaggcaaaatgccgtgtttatctcgtcaacttgttggcgagatttt

SEQ ID NO: 235 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
gctaactgaagttcctgcttcgttttagtactctggaaacagaatctactaaaacaaggcaaaatgccgtgtttatctcgtcaacttgttggcgagatttt

SEQ ID NO: 236 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
gagagcccatctggctatctcgttttagtactctggaaacagaatctactaaaacaaggcaaaatgccgtgtttatctcgtcaacttgttggcgagatttt

SEQ ID NO: 237 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
aattactggcaagtatggtgtgttttagtactctggaaacagaatctactaaaacaaggcaaaatgccgtgtttatctcgtcaacttgttggcgagatttt

SEQ ID NO: 238 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
gggtgtaggggtaggtgggctgttttagtactctggaaacagaatctactaaaacaaggcaaaatgccgtgtttatctcgtcaacttgttggcgagatttt

SEQ ID NO: 239 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
ctacacccaggagaccacgtggttttagtactctggaaacagaatctactaaaacaaggcaaaatgccgtgtttatctcgtcaacttgttggcgagatttt

SEQ ID NO: 240 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
tccacgtggtctcctgggtgtgttttagtactctggaaacagaatctactaaaacaaggcaaaatgccgtgtttatctcgtcaacttgttggcgagatttt

SEQ ID NO: 241 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
gtcgattctccacgtggtctcgttttagtactctggaaacagaatctactaaaacaaggcaaaatgccgtgtttatctcgtcaacttgttggcgagatttt

SEQ ID NO: 242 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
tggagaatcgacacagttggcgttttagtactctggaaacagaatctactaaaacaaggcaaaatgccgtgtttatctcgtcaacttgttggcgagatttt

SEQ ID NO: 243 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
gcacggatgtccgccaggtttgttttagtactctggaaacagaatctactaaaacaaggcaaaatgccgtgtttatctcgtcaacttgttggcgagatttt

SEQ ID NO: 244 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
taggcagtatgtgaaccttaggttttagtactctggaaacagaatctactaaaacaaggcaaaatgccgtgtttatctcgtcaacttgttggcgagatttt

SEQ ID NO: 245 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
ctgcctaggccactggaaagcgttttagtactctggaaacagaatctactaaaacaaggcaaaatgccgtgtttatctcgtcaacttgttggcgagatttt

SEQ ID NO: 246 - RNA, 101 nt, synthetic construct
  source 1..101: mol_type = other RNA
  misc_feature 98..101: note = phosphorothioate linkage
  misc_feature 98..101: note = 2'-O-methyl nucleotide
  misc_feature 1..3: note = 2'-O-methyl nucleotide
  misc_feature 1..4: note = phosphorothioate linkage
ggagcctctatttccagggcggttttagtactctggaaacagaatctactaaaacaaggcaaaatgccgtgtttatctcgtcaacttgttggcgagatttt

SEQ ID NO: 247 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
cgccctggaaatagaggctccgttttagtactctggaaacagaatctactaaaacaaggcaaaatgccgtgtttatctcgtcaacttgttggcgagatttt

SEQ ID NO: 248 - RNA, 101 nt, synthetic construct
  source 1..101: mol_type = other RNA
  misc_feature 98..101: note = 2'-O-methyl nucleotide
  misc_feature 98..101: note = phosphorothioate linkage
  misc_feature 1..3: note = 2'-O-methyl nucleotide
  misc_feature 1..4: note = phosphorothioate linkage
agaactgtcataagatatgaggttttagtactctggaaacagaatctactaaaacaaggcaaaatgccgtgtttatctcgtcaacttgttggcgagatttt

SEQ ID NO: 249 - RNA, 97 nt, synthetic construct
  source 1..97: mol_type = other RNA
gtgcacgttgctgcagctttgttttagagctagaaatagcaagttaaaataaggctagtccgttatcaacttgaaaaagtggcaccgagtcggtgct

SEQ ID NO: 250 - RNA, 97 nt, synthetic construct
  source 1..97: mol_type = other RNA
agctggacagctggcatctcgttttagagctagaaatagcaagttaaaataaggctagtccgttatcaacttgaaaaagtggcaccgagtcggtgct

SEQ ID NO: 251 - RNA, 97 nt, synthetic construct
  source 1..97: mol_type = other RNA
agctgtccagctgctgcttcgttttagagctagaaatagcaagttaaaataaggctagtccgttatcaacttgaaaaagtggcaccgagtcggtgct

SEQ ID NO: 252 - RNA, 97 nt, synthetic construct
  source 1..97: mol_type = other RNA
gcttctggcctgcctggtgtgttttagagctagaaatagcaagttaaaataaggctagtccgttatcaacttgaaaaagtggcaccgagtcggtgct

SEQ ID NO: 253 - RNA, 97 nt, synthetic construct
  source 1..97: mol_type = other RNA
gcctgcctggtgtgggatgtgttttagagctagaaatagcaagttaaaataaggctagtccgttatcaacttgaaaaagtggcaccgagtcggtgct

SEQ ID NO: 254 - RNA, 97 nt, synthetic construct
  source 1..97: mol_type = other RNA
ctgcctggtgtgggatgtgggttttagagctagaaatagcaagttaaaataaggctagtccgttatcaacttgaaaaagtggcaccgagtcggtgct

SEQ ID NO: 255 - RNA, 97 nt, synthetic construct
  source 1..97: mol_type = other RNA
gggccaggacagctcagctcgttttagagctagaaatagcaagttaaaataaggctagtccgttatcaacttgaaaaagtggcaccgagtcggtgct

SEQ ID NO: 256 - RNA, 97 nt, synthetic construct
  source 1..97: mol_type = other RNA
gcatcggccactctggtcatgttttagagctagaaatagcaagttaaaataaggctagtccgttatcaacttgaaaaagtggcaccgagtcggtgct

SEQ ID NO: 257 - RNA, 97 nt, synthetic construct
  source 1..97: mol_type = other RNA
tatactggcatcggccactcgttttagagctagaaatagcaagttaaaataaggctagtccgttatcaacttgaaaaagtggcaccgagtcggtgct

SEQ ID NO: 258 - RNA, 97 nt, synthetic construct
  source 1..97: mol_type = other RNA
atgccagtataccttcagtggttttagagctagaaatagcaagttaaaataaggctagtccgttatcaacttgaaaaagtggcaccgagtcggtgct

SEQ ID NO: 259 - RNA, 97 nt, synthetic construct
  source 1..97: mol_type = other RNA
cactgaaggtatactggcatgttttagagctagaaatagcaagttaaaataaggctagtccgttatcaacttgaaaaagtggcaccgagtcggtgct

SEQ ID NO: 260 - RNA, 97 nt, synthetic construct
  source 1..97: mol_type = other RNA
tggccacactgaaggtatacgttttagagctagaaatagcaagttaaaataaggctagtccgttatcaacttgaaaaagtggcaccgagtcggtgct

SEQ ID NO: 261 - RNA, 97 nt, synthetic construct
  source 1..97: mol_type = other RNA
attgggactggccacactgagttttagagctagaaatagcaagttaaaataaggctagtccgttatcaacttgaaaaagtggcaccgagtcggtgct

SEQ ID NO: 262 - RNA, 97 nt, synthetic construct
  source 1..97: mol_type = other RNA
gcagctggattcattgggacgttttagagctagaaatagcaagttaaaataaggctagtccgttatcaacttgaaaaagtggcaccgagtcggtgct

SEQ ID NO: 263 - RNA, 97 nt, synthetic construct
  source 1..97: mol_type = other RNA
tctgggcagctggattcattgttttagagctagaaatagcaagttaaaataaggctagtccgttatcaacttgaaaaagtggcaccgagtcggtgct

SEQ ID NO: 264 - RNA, 97 nt, synthetic construct
  source 1..97: mol_type = other RNA
ctctgggcagctggattcatgttttagagctagaaatagcaagttaaaataaggctagtccgttatcaacttgaaaaagtggcaccgagtcggtgct

SEQ ID NO: 265 - RNA, 97 nt, synthetic construct
  source 1..97: mol_type = other RNA
ctggctctgctctgggcagcgttttagagctagaaatagcaagttaaaataaggctagtccgttatcaacttgaaaaagtggcaccgagtcggtgct

SEQ ID NO: 266 - RNA, 97 nt, synthetic construct
  source 1..97: mol_type = other RNA
taagttatggatgactgacagttttagagctagaaatagcaagttaaaataaggctagtccgttatcaacttgaaaaagtggcaccgagtcggtgct

SEQ ID NO: 267 - RNA, 97 nt, synthetic construct
  source 1..97: mol_type = other RNA
cagcacccaacgcttagaccgttttagagctagaaatagcaagttaaaataaggctagtccgttatcaacttgaaaaagtggcaccgagtcggtgct

SEQ ID NO: 268 - RNA, 97 nt, synthetic construct
  source 1..97: mol_type = other RNA
cacccaacgcttagacctgggttttagagctagaaatagcaagttaaaataaggctagtccgttatcaacttgaaaaagtggcaccgagtcggtgct

SEQ ID NO: 269 - RNA, 97 nt, synthetic construct
  source 1..97: mol_type = other RNA
ggcctccaggtctaagcgttgttttagagctagaaatagcaagttaaaataaggctagtccgttatcaacttgaaaaagtggcaccgagtcggtgct

SEQ ID NO: 270 - RNA, 97 nt, synthetic construct
  source 1..97: mol_type = other RNA
tggcctccaggtctaagcgtgttttagagctagaaatagcaagttaaaataaggctagtccgttatcaacttgaaaaagtggcaccgagtcggtgct

SEQ ID NO: 271 - RNA, 97 nt, synthetic construct
  source 1..97: mol_type = other RNA
gtcgagctttggtggcctccgttttagagctagaaatagcaagttaaaataaggctagtccgttatcaacttgaaaaagtggcaccgagtcggtgct

SEQ ID NO: 272 - RNA, 97 nt, synthetic construct
  source 1..97: mol_type = other RNA
ggagctgagtcgagctttgggttttagagctagaaatagcaagttaaaataaggctagtccgttatcaacttgaaaaagtggcaccgagtcggtgct

SEQ ID NO: 273 - RNA, 97 nt, synthetic construct
  source 1..97: mol_type = other RNA
caaagctcgactcagctcccgttttagagctagaaatagcaagttaaaataaggctagtccgttatcaacttgaaaaagtggcaccgagtcggtgct

SEQ ID NO: 274 - RNA, 97 nt, synthetic construct
  source 1..97: mol_type = other RNA
cagggagctgagtcgagcttgttttagagctagaaatagcaagttaaaataaggctagtccgttatcaacttgaaaaagtggcaccgagtcggtgct

SEQ ID NO: 275 - RNA, 97 nt, synthetic construct
  source 1..97: mol_type = other RNA
ttggtggaggaggctctccagttttagagctagaaatagcaagttaaaataaggctagtccgttatcaacttgaaaaagtggcaccgagtcggtgct

SEQ ID NO: 276 - RNA, 97 nt, synthetic construct
  source 1..97: mol_type = other RNA
attggtggaggaggctctccgttttagagctagaaatagcaagttaaaataaggctagtccgttatcaacttgaaaaagtggcaccgagtcggtgct

SEQ ID NO: 277 - RNA, 97 nt, synthetic construct
  source 1..97: mol_type = other RNA
cctcctccaccaattgacctgttttagagctagaaatagcaagttaaaataaggctagtccgttatcaacttgaaaaagtggcaccgagtcggtgct

SEQ ID NO: 278 - RNA, 97 nt, synthetic construct
  source 1..97: mol_type = other RNA
ccaaggtcaattggtggagggttttagagctagaaatagcaagttaaaataaggctagtccgttatcaacttgaaaaagtggcaccgagtcggtgct

SEQ ID NO: 279 - RNA, 97 nt, synthetic construct
  source 1..97: mol_type = other RNA
ccaccaattgaccttggaccgttttagagctagaaatagcaagttaaaataaggctagtccgttatcaacttgaaaaagtggcaccgagtcggtgct

SEQ ID NO: 280 - RNA, 97 nt, synthetic construct
  source 1..97: mol_type = other RNA
ggtccaaggtcaattggtgggttttagagctagaaatagcaagttaaaataaggctagtccgttatcaacttgaaaaagtggcaccgagtcggtgct

SEQ ID NO: 281 - RNA, 97 nt, synthetic construct
  source 1..97: mol_type = other RNA
cctggtccaaggtcaattgggttttagagctagaaatagcaagttaaaataaggctagtccgttatcaacttgaaaaagtggcaccgagtcggtgct

SEQ ID NO: 282 - RNA, 97 nt, synthetic construct
  source 1..97: mol_type = other RNA
cagcctggtccaaggtcaatgttttagagctagaaatagcaagttaaaataaggctagtccgttatcaacttgaaaaagtggcaccgagtcggtgct

SEQ ID NO: 283 - RNA, 97 nt, synthetic construct
  source 1..97: mol_type = other RNA
gggcaccctgaggcgggagcgttttagagctagaaatagcaagttaaaataaggctagtccgttatcaacttgaaaaagtggcaccgagtcggtgct

SEQ ID NO: 284 - RNA, 97 nt, synthetic construct
  source 1..97: mol_type = other RNA
ctggtcccgctcccgcctcagttttagagctagaaatagcaagttaaaataaggctagtccgttatcaacttgaaaaagtggcaccgagtcggtgct

SEQ ID NO: 285 - RNA, 97 nt, synthetic construct
  source 1..97: mol_type = other RNA
gctggtcccgctcccgcctcgttttagagctagaaatagcaagttaaaataaggctagtccgttatcaacttgaaaaagtggcaccgagtcggtgct

SEQ ID NO: 286 - RNA, 97 nt, synthetic construct
  source 1..97: mol_type = other RNA
gaggcgggagcgggaccagcgttttagagctagaaatagcaagttaaaataaggctagtccgttatcaacttgaaaaagtggcaccgagtcggtgct

SEQ ID NO: 287 - RNA, 97 nt, synthetic construct
  source 1..97: mol_type = other RNA
ctctggtttgggtttccagcgttttagagctagaaatagcaagttaaaataaggctagtccgttatcaacttgaaaaagtggcaccgagtcggtgct

SEQ ID NO: 288 - RNA, 97 nt, synthetic construct
  source 1..97: mol_type = other RNA
agtctccaactctctggtttgttttagagctagaaatagcaagttaaaataaggctagtccgttatcaacttgaaaaagtggcaccgagtcggtgct

SEQ ID NO: 289 - RNA, 97 nt, synthetic construct
  source 1..97: mol_type = other RNA
gtctcggaggaggttgctgtgttttagagctagaaatagcaagttaaaataaggctagtccgttatcaacttgaaaaagtggcaccgagtcggtgct

SEQ ID NO: 290 - RNA, 97 nt, synthetic construct
  source 1..97: mol_type = other RNA
cctccgagacaagtcagttcgttttagagctagaaatagcaagttaaaataaggctagtccgttatcaacttgaaaaagtggcaccgagtcggtgct

SEQ ID NO: 291 - RNA, 97 nt, synthetic construct
  source 1..97: mol_type = other RNA
gaactgacttgtctcggagggttttagagctagaaatagcaagttaaaataaggctagtccgttatcaacttgaaaaagtggcaccgagtcggtgct

SEQ ID NO: 292 - RNA, 97 nt, synthetic construct
  source 1..97: mol_type = other RNA
ccagaactgacttgtctcgggttttagagctagaaatagcaagttaaaataaggctagtccgttatcaacttgaaaaagtggcaccgagtcggtgct

SEQ ID NO: 293 - RNA, 97 nt, synthetic construct
  source 1..97: mol_type = other RNA
ccgagacaagtcagttctgggttttagagctagaaatagcaagttaaaataaggctagtccgttatcaacttgaaaaagtggcaccgagtcggtgct

SEQ ID NO: 294 - RNA, 97 nt, synthetic construct
  source 1..97: mol_type = other RNA
cctccagaactgacttgtctgttttagagctagaaatagcaagttaaaataaggctagtccgttatcaacttgaaaaagtggcaccgagtcggtgct

SEQ ID NO: 295 - RNA, 97 nt, synthetic construct
  source 1..97: mol_type = other RNA
aggaagagaagaagcgactagttttagagctagaaatagcaagttaaaataaggctagtccgttatcaacttgaaaaagtggcaccgagtcggtgct

SEQ ID NO: 296 - RNA, 97 nt, synthetic construct
  source 1..97: mol_type = other RNA
aagaaaatgagaatctggccgttttagagctagaaatagcaagttaaaataaggctagtccgttatcaacttgaaaaagtggcaccgagtcggtgct

SEQ ID NO: 297 - RNA, 97 nt, synthetic construct
  source 1..97: mol_type = other RNA
tgagaatctggccaggaggtgttttagagctagaaatagcaagttaaaataaggctagtccgttatcaacttgaaaaagtggcaccgagtcggtgct

SEQ ID NO: 298 - RNA, 97 nt, synthetic construct
  source 1..97: mol_type = other RNA
gctgctgctttccaacctccgttttagagctagaaatagcaagttaaaataaggctagtccgttatcaacttgaaaaagtggcaccgagtcggtgct

SEQ ID NO: 299 - RNA, 97 nt, synthetic construct
  source 1..97: mol_type = other RNA
gaggttggaaagcagcagccgttttagagctagaaatagcaagttaaaataaggctagtccgttatcaacttgaaaaagtggcaccgagtcggtgct

SEQ ID NO: 300 - RNA, 97 nt, synthetic construct
  source 1..97: mol_type = other RNA
gttggaaagcagcagccagggttttagagctagaaatagcaagttaaaataaggctagtccgttatcaacttgaaaaagtggcaccgagtcggtgct

SEQ ID NO: 301 - RNA, 97 nt, synthetic construct
  source 1..97: mol_type = other RNA
gcagcagccaggaggtagcagttttagagctagaaatagcaagttaaaataaggctagtccgttatcaacttgaaaaagtggcaccgagtcggtgct

SEQ ID NO: 302 - RNA, 97 nt, synthetic construct
  source 1..97: mol_type = other RNA
tgtctcgggtctggggacacgttttagagctagaaatagcaagttaaaataaggctagtccgttatcaacttgaaaaagtggcaccgagtcggtgct

SEQ ID NO: 303 - RNA, 97 nt, synthetic construct
  source 1..97: mol_type = other RNA
ccagacccgagacactgctcgttttagagctagaaatagcaagttaaaataaggctagtccgttatcaacttgaaaaagtggcaccgagtcggtgct

SEQ ID NO: 304 - RNA, 97 nt, synthetic construct
  source 1..97: mol_type = other RNA
cgagcagtgtctcgggtctggttttagagctagaaatagcaagttaaaataaggctagtccgttatcaacttgaaaaagtggcaccgagtcggtgct

SEQ ID NO: 305 - RNA, 97 nt, synthetic construct
  source 1..97: mol_type = other RNA
ccgagcagtgtctcgggtctgttttagagctagaaatagcaagttaaaataaggctagtccgttatcaacttgaaaaagtggcaccgagtcggtgct

SEQ ID NO: 306 - RNA, 97 nt, synthetic construct
  source 1..97: mol_type = other RNA
cccgagcagtgtctcgggtcgttttagagctagaaatagcaagttaaaataaggctagtccgttatcaacttgaaaaagtggcaccgagtcggtgct

SEQ ID NO: 307 - RNA, 97 nt, synthetic construct
  source 1..97: mol_type = other RNA
cacagcccgagcagtgtctcgttttagagctagaaatagcaagttaaaataaggctagtccgttatcaacttgaaaaagtggcaccgagtcggtgct

SEQ ID NO: 308 - RNA, 97 nt, synthetic construct
  source 1..97: mol_type = other RNA
gcacagcccgagcagtgtctgttttagagctagaaatagcaagttaaaataaggctagtccgttatcaacttgaaaaagtggcaccgagtcggtgct

SEQ ID NO: 309 - RNA, 97 nt, synthetic construct
  source 1..97: mol_type = other RNA
actgctcgggctgtgccaccgttttagagctagaaatagcaagttaaaataaggctagtccgttatcaacttgaaaaagtggcaccgagtcggtgct

SEQ ID NO: 310 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..3: note = 2'-O-methyl nucleotide
  misc_feature 1..4: note = phosphorothioate linkage
gtgcacgttgctgcagctttgttttagagctagaaatagcaagttaaaataaggctagtccgttatcaacttgaaaaagtggcaccgagtcggtgctttt

SEQ ID NO: 311 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
agctggacagctggcatctcgttttagagctagaaatagcaagttaaaataaggctagtccgttatcaacttgaaaaagtggcaccgagtcggtgctttt

SEQ ID NO: 312 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
agctgtccagctgctgcttcgttttagagctagaaatagcaagttaaaataaggctagtccgttatcaacttgaaaaagtggcaccgagtcggtgctttt

SEQ ID NO: 313 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gcttctggcctgcctggtgtgttttagagctagaaatagcaagttaaaataaggctagtccgttatcaacttgaaaaagtggcaccgagtcggtgctttt

SEQ ID NO: 314 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gcctgcctggtgtgggatgtgttttagagctagaaatagcaagttaaaataaggctagtccgttatcaacttgaaaaagtggcaccgagtcggtgctttt

SEQ ID NO: 315 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ctgcctggtgtgggatgtgggttttagagctagaaatagcaagttaaaataaggctagtccgttatcaacttgaaaaagtggcaccgagtcggtgctttt

SEQ ID NO: 316 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gggccaggacagctcagctcgttttagagctagaaatagcaagttaaaataaggctagtccgttatcaacttgaaaaagtggcaccgagtcggtgctttt

SEQ ID NO: 317 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gcatcggccactctggtcatgttttagagctagaaatagcaagttaaaataaggctagtccgttatcaacttgaaaaagtggcaccgagtcggtgctttt

SEQ ID NO: 318 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
tatactggcatcggccactcgttttagagctagaaatagcaagttaaaataaggctagtccgttatcaacttgaaaaagtggcaccgagtcggtgctttt

SEQ ID NO: 319 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
atgccagtataccttcagtggttttagagctagaaatagcaagttaaaataaggctagtccgttatcaacttgaaaaagtggcaccgagtcggtgctttt

SEQ ID NO: 320 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
cactgaaggtatactggcatgttttagagctagaaatagcaagttaaaataaggctagtccgttatcaacttgaaaaagtggcaccgagtcggtgctttt

SEQ ID NO: 321 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
tggccacactgaaggtatacgttttagagctagaaatagcaagttaaaataaggctagtccgttatcaacttgaaaaagtggcaccgagtcggtgctttt

SEQ ID NO: 322 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
attgggactggccacactgagttttagagctagaaatagcaagttaaaataaggctagtccgttatcaacttgaaaaagtggcaccgagtcggtgctttt

SEQ ID NO: 323 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gcagctggattcattgggacgttttagagctagaaatagcaagttaaaataaggctagtccgttatcaacttgaaaaagtggcaccgagtcggtgctttt

SEQ ID NO: 324 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
tctgggcagctggattcattgttttagagctagaaatagcaagttaaaataaggctagtccgttatcaacttgaaaaagtggcaccgagtcggtgctttt

SEQ ID NO: 325 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ctctgggcagctggattcatgttttagagctagaaatagcaagttaaaataaggctagtccgttatcaacttgaaaaagtggcaccgagtcggtgctttt

SEQ ID NO: 326 - RNA, 100 nt, synthetic construct
  source 1..100: mol_type = other RNA
  misc_feature 97..100: note = phosphorothioate linkage
  misc_feature 97..100: note = 2'-O-methyl nucleotide
  misc_feature 1..4: note = phosphorothioate linkage
  misc_feature 1..3: note = 2'-O-methyl nucleotide
ctggctctgctctgggcagcgttttagagctagaaatagcaagttaaaataaggctagtccgttatcaacttgaaaaagtggcaccgagtcggtgctttt

SEQ ID NO: 327 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
taagttatggatgactgacagttttagagctagaaatagcaagttaaaataaggctagtccgttatcaacttgaaaaagtggcaccgagtcggtgctttt

SEQ ID NO: 328 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
cagcacccaacgcttagaccgttttagagctagaaatagcaagttaaaataaggctagtccgttatcaacttgaaaaagtggcaccgagtcggtgctttt

SEQ ID NO: 329 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
cacccaacgcttagacctgggttttagagctagaaatagcaagttaaaataaggctagtccgttatcaacttgaaaaagtggcaccgagtcggtgctttt

SEQ ID NO: 330 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ggcctccaggtctaagcgttgttttagagctagaaatagcaagttaaaataaggctagtccgttatcaacttgaaaaagtggcaccgagtcggtgctttt

SEQ ID NO: 331 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..3: note = 2'-O-methyl nucleotide
  misc_feature 1..4: note = phosphorothioate linkage
tggcctccaggtctaagcgtgttttagagctagaaatagcaagttaaaataaggctagtccgttatcaacttgaaaaagtggcaccgagtcggtgctttt

SEQ ID NO: 332 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gtcgagctttggtggcctccgttttagagctagaaatagcaagttaaaataaggctagtccgttatcaacttgaaaaagtggcaccgagtcggtgctttt

SEQ ID NO: 333 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ggagctgagtcgagctttgggttttagagctagaaatagcaagttaaaataaggctagtccgttatcaacttgaaaaagtggcaccgagtcggtgctttt

SEQ ID NO: 334 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
caaagctcgactcagctcccgttttagagctagaaatagcaagttaaaataaggctagtccgttatcaacttgaaaaagtggcaccgagtcggtgctttt

SEQ ID NO: 335 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
cagggagctgagtcgagcttgttttagagctagaaatagcaagttaaaataaggctagtccgttatcaacttgaaaaagtggcaccgagtcggtgctttt

SEQ ID NO: 336 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ttggtggaggaggctctccagttttagagctagaaatagcaagttaaaataaggctagtccgttatcaacttgaaaaagtggcaccgagtcggtgctttt

SEQ ID NO: 337 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
attggtggaggaggctctccgttttagagctagaaatagcaagttaaaataaggctagtccgttatcaacttgaaaaagtggcaccgagtcggtgctttt

SEQ ID NO: 338 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
cctcctccaccaattgacctgttttagagctagaaatagcaagttaaaataaggctagtccgttatcaacttgaaaaagtggcaccgagtcggtgctttt

SEQ ID NO: 339 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ccaaggtcaattggtggagggttttagagctagaaatagcaagttaaaataaggctagtccgttatcaacttgaaaaagtggcaccgagtcggtgctttt

SEQ ID NO: 340 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ccaccaattgaccttggaccgttttagagctagaaatagcaagttaaaataaggctagtccgttatcaacttgaaaaagtggcaccgagtcggtgctttt

SEQ ID NO: 341 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ggtccaaggtcaattggtgggttttagagctagaaatagcaagttaaaataaggctagtccgttatcaacttgaaaaagtggcaccgagtcggtgctttt

SEQ ID NO: 342 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
cctggtccaaggtcaattgggttttagagctagaaatagcaagttaaaataaggctagtccgttatcaacttgaaaaagtggcaccgagtcggtgctttt

SEQ ID NO: 343 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
cagcctggtccaaggtcaatgttttagagctagaaatagcaagttaaaataaggctagtccgttatcaacttgaaaaagtggcaccgagtcggtgctttt

SEQ ID NO: 344 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gggcaccctgaggcgggagcgttttagagctagaaatagcaagttaaaataaggctagtccgttatcaacttgaaaaagtggcaccgagtcggtgctttt

SEQ ID NO: 345 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ctggtcccgctcccgcctcagttttagagctagaaatagcaagttaaaataaggctagtccgttatcaacttgaaaaagtggcaccgagtcggtgctttt

SEQ ID NO: 346 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gctggtcccgctcccgcctcgttttagagctagaaatagcaagttaaaataaggctagtccgttatcaacttgaaaaagtggcaccgagtcggtgctttt

SEQ ID NO: 347 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gaggcgggagcgggaccagcgttttagagctagaaatagcaagttaaaataaggctagtccgttatcaacttgaaaaagtggcaccgagtcggtgctttt

SEQ ID NO: 348 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ctctggtttgggtttccagcgttttagagctagaaatagcaagttaaaataaggctagtccgttatcaacttgaaaaagtggcaccgagtcggtgctttt

SEQ ID NO: 349 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
agtctccaactctctggtttgttttagagctagaaatagcaagttaaaataaggctagtccgttatcaacttgaaaaagtggcaccgagtcggtgctttt

SEQ ID NO: 350 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gtctcggaggaggttgctgtgttttagagctagaaatagcaagttaaaataaggctagtccgttatcaacttgaaaaagtggcaccgagtcggtgctttt

SEQ ID NO: 351 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
cctccgagacaagtcagttcgttttagagctagaaatagcaagttaaaataaggctagtccgttatcaacttgaaaaagtggcaccgagtcggtgctttt

SEQ ID NO: 352 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gaactgacttgtctcggagggttttagagctagaaatagcaagttaaaataaggctagtccgttatcaacttgaaaaagtggcaccgagtcggtgctttt

SEQ ID NO: 353 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ccagaactgacttgtctcgggttttagagctagaaatagcaagttaaaataaggctagtccgttatcaacttgaaaaagtggcaccgagtcggtgctttt

SEQ ID NO: 354 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
ccgagacaagtcagttctgggttttagagctagaaatagcaagttaaaataaggctagtccgttatcaacttgaaaaagtggcaccgagtcggtgctttt

SEQ ID NO: 355 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
cctccagaactgacttgtctgttttagagctagaaatagcaagttaaaataaggctagtccgttatcaacttgaaaaagtggcaccgagtcggtgctttt

SEQ ID NO: 356 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
aggaagagaagaagcgactagttttagagctagaaatagcaagttaaaataaggctagtccgttatcaacttgaaaaagtggcaccgagtcggtgctttt

SEQ ID NO: 357 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
aagaaaatgagaatctggccgttttagagctagaaatagcaagttaaaataaggctagtccgttatcaacttgaaaaagtggcaccgagtcggtgctttt

SEQ ID NO: 358 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
tgagaatctggccaggaggtgttttagagctagaaatagcaagttaaaataaggctagtccgttatcaacttgaaaaagtggcaccgagtcggtgctttt

SEQ ID NO: 359 - RNA, 100 nt, synthetic construct
  source 1..100: mol_type = other RNA
  misc_feature 97..100: note = 2'-O-methyl nucleotide
  misc_feature 97..100: note = phosphorothioate linkage
  misc_feature 1..4: note = phosphorothioate linkage
  misc_feature 1..3: note = 2'-O-methyl nucleotide
gctgctgctttccaacctccgttttagagctagaaatagcaagttaaaataaggctagtccgttatcaacttgaaaaagtggcaccgagtcggtgctttt

SEQ ID NO: 360 - RNA, 100 nt, synthetic construct
  source 1..100: mol_type = other RNA
  misc_feature 97..100: note = phosphorothioate linkage
  misc_feature 97..100: note = 2'-O-methyl nucleotide
  misc_feature 1..4: note = phosphorothioate linkage
  misc_feature 1..3: note = 2'-O-methyl nucleotide
gaggttggaaagcagcagccgttttagagctagaaatagcaagttaaaataaggctagtccgttatcaacttgaaaaagtggcaccgagtcggtgctttt

SEQ ID NO: 361 - RNA, 100 nt, synthetic construct
  source 1..100: mol_type = other RNA
  misc_feature 97..100: note = 2'-O-methyl nucleotide
  misc_feature 97..100: note = Phosphorothioate linkage
  misc_feature 1..4: note = phosphorothioate linkage
  misc_feature 1..3: note = 2'-O-methyl nucleotide
gttggaaagcagcagccagggttttagagctagaaatagcaagttaaaataaggctagtccgttatcaacttgaaaaagtggcaccgagtcggtgctttt

SEQ ID NO: 362 - RNA, 100 nt, synthetic construct
  source 1..100: mol_type = other RNA
  misc_feature 97..100: note = 2'-O-methyl nucleotide
  misc_feature 97..100: note = Phosphorothioate linkage
  misc_feature 1..4: note = phosphorothioate linkage
  misc_feature 1..3: note = 2'-O-methyl nucleotide
gcagcagccaggaggtagcagttttagagctagaaatagcaagttaaaataaggctagtccgttatcaacttgaaaaagtggcaccgagtcggtgctttt

SEQ ID NO: 363 - RNA, 100 nt, synthetic construct
  source 1..100: mol_type = other RNA
  misc_feature 97..100: note = 2'-O-methyl nucleotide
  misc_feature 97..100: note = Phosphorothioate linkage
  misc_feature 1..4: note = phosphorothioate linkage
  misc_feature 1..3: note = 2'-O-methyl nucleotide
tgtctcgggtctggggacacgttttagagctagaaatagcaagttaaaataaggctagtccgttatcaacttgaaaaagtggcaccgagtcggtgctttt

SEQ ID NO: 364 - RNA, 100 nt, synthetic construct
  source 1..100: mol_type = other RNA
  misc_feature 97..100: note = 2'-O-methyl nucleotide
  misc_feature 97..100: note = Phosphorothioate linkage
  misc_feature 1..4: note = phosphorothioate linkage
  misc_feature 1..3: note = 2'-O-methyl nucleotide
ccagacccgagacactgctcgttttagagctagaaatagcaagttaaaataaggctagtccgttatcaacttgaaaaagtggcaccgagtcggtgctttt

SEQ ID NO: 365 - RNA, 100 nt, synthetic construct
  source 1..100: mol_type = other RNA
  misc_feature 97..100: note = 2'-O-methyl nucleotide
  misc_feature 97..100: note = Phosphorothioate linkage
  misc_feature 1..4: note = phosphorothioate linkage
  misc_feature 1..3: note = 2'-O-methyl nucleotide
cgagcagtgtctcgggtctggttttagagctagaaatagcaagttaaaataaggctagtccgttatcaacttgaaaaagtggcaccgagtcggtgctttt

SEQ ID NO: 366 - RNA, 100 nt, synthetic construct
  source 1..100: mol_type = other RNA
  misc_feature 97..100: note = 2'-O-methyl nucleotide
  misc_feature 97..100: note = Phosphorothioate linkage
  misc_feature 1..4: note = phosphorothioate linkage
  misc_feature 1..3: note = 2'-O-methyl nucleotide
ccgagcagtgtctcgggtctgttttagagctagaaatagcaagttaaaataaggctagtccgttatcaacttgaaaaagtggcaccgagtcggtgctttt

SEQ ID NO: 367 - RNA, 100 nt, synthetic construct
  source 1..100: mol_type = other RNA
  misc_feature 97..100: note = 2'-O-methyl nucleotide
  misc_feature 97..100: note = Phosphorothioate linkage
  misc_feature 1..4: note = phosphorothioate linkage
  misc_feature 1..3: note = 2'-O-methyl nucleotide
cccgagcagtgtctcgggtcgttttagagctagaaatagcaagttaaaataaggctagtccgttatcaacttgaaaaagtggcaccgagtcggtgctttt

SEQ ID NO: 368 - RNA, 100 nt, synthetic construct
  source 1..100: mol_type = other RNA
  misc_feature 97..100: note = 2'-O-methyl nucleotide
  misc_feature 97..100: note = Phosphorothioate linkage
  misc_feature 1..4: note = phosphorothioate linkage
  misc_feature 1..3: note = 2'-O-methyl nucleotide
cacagcccgagcagtgtctcgttttagagctagaaatagcaagttaaaataaggctagtccgttatcaacttgaaaaagtggcaccgagtcggtgctttt

SEQ ID NO: 369 - RNA, 100 nt, synthetic construct
  source 1..100: mol_type = other RNA
  misc_feature 97..100: note = 2'-O-methyl nucleotide
  misc_feature 97..100: note = Phosphorothioate linkage
  misc_feature 1..4: note = phosphorothioate linkage
  misc_feature 1..3: note = 2'-O-methyl nucleotide
gcacagcccgagcagtgtctgttttagagctagaaatagcaagttaaaataaggctagtccgttatcaacttgaaaaagtggcaccgagtcggtgctttt

SEQ ID NO: 370 - RNA, 100 nt, synthetic construct
  source 1..100: mol_type = other RNA
  misc_feature 97..100: note = 2'-O-methyl nucleotide
  misc_feature 97..100: note = Phosphorothioate linkage
  misc_feature 1..4: note = phosphorothioate linkage
  misc_feature 1..3: note = 2'-O-methyl nucleotide
actgctcgggctgtgccaccgttttagagctagaaatagcaagttaaaataaggctagtccgttatcaacttgaaaaagtggcaccgagtcggtgctttt

SEQ ID NO: 371 - RNA, 100 nt, synthetic construct; identical to SEQ ID NO: 319
  source 1..100: mol_type = other RNA
  misc_feature 1..4: note = Phosphorothioate linkage
  misc_feature 97..100: note = Phosphorothioate linkage
  misc_feature 1..3: note = 2'-O-methyl nucleotide
  misc_feature 29..40: note = 2'-O-methyl nucleotide
  misc_feature 68..100: note = 2'-O-methyl nucleotide